(19) United States
(12) Patent Application Publication
(10) Pub. No.: US 2024/0018166 A1
(43) Pub. Date: Jan. 18, 2024

(54) SULFONYL UREA NLRP3 INFLAMMASOME INHIBITORS

(71) Applicant: Venatorx Pharmaceuticals, Inc., Malvern, PA (US)

(72) Inventors: Bin LIU, Plainsboro, NJ (US); Jiangchao YAO, Princeton, NJ (US); Thomas HAIMOWITZ, Newton, PA (US); Christopher BENETATOS, Chester Springs, PA (US); Stephen M. CONDON, Glenmoore, PA (US); Steven A. BOYD, Chester Springs, PA (US)

(21) Appl. No.: 18/254,031

(22) PCT Filed: Nov. 23, 2021
(86) PCT No.: PCT/US2021/060487

§ 371 Date: May 23, 2023

Related U.S. Application Data
(60) Provisional application No. 63/118,397, filed on Nov. 25, 2020.

(51) Int. Cl. C07F 5/02; C07F 5/04

(52) U.S. Cl. CPC C07F 5/025; C07F 5/04

(57) ABSTRACT

Disclosed herein are NLRP3 inflammasome inhibitors and compositions thereof. Also disclosed herein are methods including administering a therapeutically effective amount of a composition comprising at least one NLRP3 inflammasome inhibitor to a subject in need thereof.

### CROSS-REFERENCE

This application claims the benefit of U.S. Provisional Application Ser. No. 63/118,397 filed Nov. 25, 2020 which is hereby incorporated by reference in its entirety.

### BACKGROUND OF THE INVENTION

Inflammasomes are intracellular protein complexes that mediate inflammatory signaling cascades, including nucleotide oligomerization domain (NOD)-like receptor protein (NLR) family members NLRP1, NLRP3, and NLRC4, along with other non-NLR receptors such as AIM2 and IFI16. The inappropriate activation of the NLRP3 inflammasome has been implicated in a wide range of diseases including Alzheimer's disease, Prion disease, type 2 diabetes, gout, atherosclerosis, Parkinson's disease, rheumatoid arthritis, NASH, and some infectious diseases. NLRP3 activation involves two successive signals. The first step comprises an initiating signal (priming) in which many danger associated molecular patterns (DAMPs) and pathogen associated molecular patterns (PAMPs) are recognized by TLRs, which in turn up-regulates transcription of inflammasome related components, including inactive NLRP3, pro-IL-10, and pro-IL-18. In the second step of inflammasome activation, NEK7-licensed NLRP3 binds to apoptosis associated speck-like protein containing a CARD (ASC), which, in turn interacts with pro-caspase-1, the inactive form of the cysteine protease caspase-1, thus forming the NLRP3 inflammasome. Clustering of pro-caspase-1 onto ASC results in the homolytic activation of caspase-1, which then cleaves the pro-inflammatory cytokines pro-IL-10 and pro-IL-18 to their active forms (i.e., IL-10, IL-18, respectively). In the presence of gasdermin D (GSDMD) and phosphatidylinositol (PI), NLRP3 activation results in pyroptosis, a type of inflammatory cell death characterized by cell swelling and lysis.

The inherited Cryopyrin-associated periodic syndromes (CAPS), Muckle-Wells syndrome (MWS), familial cold autoinflammatory syndrome (FCAS) and neonatal onset multi-system inflammatory disease (NOMID), are caused by gain of function mutations in NLRP3, thus implicating NLRP3 as a critical component of the inflammatory process. The NLRP3-mediated inflammatory response has been observed in rare periodic fever syndrome, CAPS, TRAPS, and a variety of human diseases such as multiple sclerosis, atherosclerosis, Alzheimer's disease, diabetes, asthma, gouty arthritis, inflammatory bowel disease (IBD), juvenile arthritis, and neurodegenerative and autoimmune diseases. Hence, the NLPR3 inflammasome is a target for the potential treatment of these complex diseases.

Current treatments for NLRP3-related diseases include biologic agents that target IL-1 such as the recombinant IL-1 receptor antagonist anakinra, the neutralizing IL-10 antibody canakinumab and the soluble decoy IL-1 receptor rilonacept. These biological agents have proven successful for the treatment of CAPS and have advanced to clinical trials for other IL-10-associated diseases. Recently, the 10,000 patient CANTOS trial of anti-IL-1 canakinumab (Novartis, 2017) revitalized interest in anti-inflammatory drugs in cancer and cardiovascular disease. In a mid-dose arm, patients experienced a 15% lower risk of non-fatal myocardial infarction, non-fatal stroke or cardiovascular death compared to placebo. However, a higher incidence of fatal infection highlighted the risk associated with pan-IL-1 blockade. In addition, high-dose canakinumab was associated with a >50% reduction in total cancer mortality (mostly lung cancer) compared to placebo. This result was in line with previous work suggesting that IL-10 was associated with increased angiogenesis and tumor growth.

Several small molecule inhibitors of the NLRP3 inflammasome previously have been described. Some diarylsulfonylurea-containing compounds including Glyburide and MCC950 have been reported as small molecule inhibitors of NLRP3 inflammasome. Recently, the binding interactions between MCC950 and the NLRP3 NACHT domain were described (Coll R, et al. “MCC950 directly targets the NLRP3 ATP hydrolysis motif for inflammasome inhibition,” Nat Chem Biol 2019, 15, 556-559; and, Tapia-Abellin A, et al. “MCC950 closes the active conformation of NLRP3 to an inactive state,” Nat Chem Biol 2019, 15, 560-564). Although potent in cellular assays for NLRP3 inhibition and IL-10 secretion, MCC950 loses much of its activity in the presence of plasma proteins or whole blood. No small molecule inhibitors of the NLRP3 inflammasome have been approved. There is a need for new NLRP3 inhibitors for the treatment of NLRP3- or IL-1β-driven inflammation.

### SUMMARY OF THE INVENTION

Disclosed herein is a compound of Formula (I), or a pharmaceutically acceptable salt, solvate, or stereoisomer thereof:

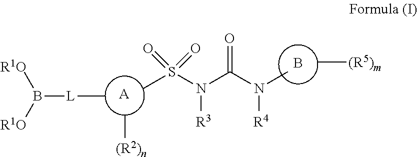

wherein:
Ring A is cycloalkyl, heterocycloalkyl, aryl, or heteroaryl;
L is a bond or a C₁-C₆ alkylene optionally substituted with one, two, or three R^{L};
each R^{L} is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{L} on the same carbon are taken together to form an oxo;
each R¹ is independently hydrogen or C₁-C₆ alkyl;
or two R¹ are taken together to form a heterocycloalkyl optionally substituted with one, two, three, four, or five Ria;
each R^{1a} is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₁-C₆alkylene(COOH), C₂-C₆alkenyl, C₂-C₆alkynyl, cycloalkyl, heterocycloalkyl, aryl, or heteroaryl;
or two R^{1a} on the same carbon are taken together to form an oxo;
each R² is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally substituted with one, two, or three R^{2a};
each R^{2a} is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{2a} on the same carbon are taken together to form an oxo;
n is 0-3;
or one R¹ and one R² are taken together to form a heterocycloalkyl optionally substituted with one, two, or three R¹⁻²;
each R¹⁻² is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R¹⁻² on the same carbon are taken together to form an oxo;
R³ and R⁴ are each independently hydrogen or C₁-C₆ alkyl;
Ring B is cycloalkyl, heterocycloalkyl, aryl, or heteroaryl;
each R⁵ is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally substituted with one, two, or three R^{a};
or two R⁵ on adjacent atoms are taken together to form a cycloalkyl, heterocycloalkyl, aryl, or heteroaryl; each optionally substituted with one, two, or three R^{5b};
each R^{5a} is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{5a} on the same carbon are taken together to form an oxo;
each R^{5b} is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{5b} on the same carbon are taken together to form an oxo;
m is 0-5;
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl);
wherein each alkyl, alkenyl, alkynyl, cycloalkyl, heterocycloalkyl, aryl, and heteroaryl is independently optionally substituted with one, two, or three deuterium, oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆deuteroalkyl, C₁-C₆haloalkyl, C₁-C₆hydroxyalkyl, or C₁-C₆aminoalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl);
wherein each alkyl, alkenyl, alkynyl, cycloalkyl, heterocycloalkyl, aryl, and heteroaryl is independently optionally substituted with one, two, or three deuterium, oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆deuteroalkyl, C₁-C₆hydroxyalkyl, or C₁-C₆aminoalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one, two, or three oxo, deuterium, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆deuteroalkyl, C₁-C₆hydroxyalkyl, or C₁-C₆aminoalkyl;
or R^{c} and R^{d} are taken together with the atom to which they are attached to form a heterocycloalkyl optionally substituted with one, two, or three oxo, deuterium, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆deuteroalkyl, C₁-C₆hydroxyalkyl, or C₁-C₆aminoalkyl.

Also disclosed herein is a compound of Formula (II), or a pharmaceutically acceptable salt, solvate, or stereoisomer thereof:

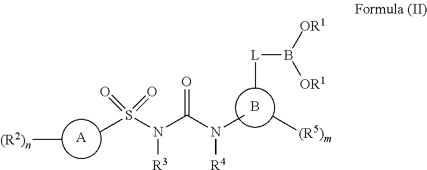

wherein:
Ring A is cycloalkyl, heterocycloalkyl, aryl, or heteroaryl;
each R² is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally substituted with one, two, or three R^{2a};
each R^{2a} is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{2a} on the same carbon are taken together to form an oxo;
n is 0-3;
R³ and R⁴ are each independently hydrogen or C₁-C₆ alkyl;
Ring B is cycloalkyl, heterocycloalkyl, aryl, or heteroaryl;
L is a bond or a C₁-C₆ alkylene optionally substituted with one, two, or three R^{L};
each R^{L} is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{L} on the same carbon are taken together to form an oxo;
each R¹ is independently hydrogen or C₁-C₆ alkyl;
or two R¹ are taken together to form a heterocycloalkyl optionally substituted with one, two, three, four, or five R^{1a};
each R^{1a} is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₁-C₆alkylene(COOH), C₂-C₆alkenyl, C₂-C₆alkynyl, cycloalkyl, heterocycloalkyl, aryl, or heteroaryl;
or two R^{1a} on the same carbon are taken together to form an oxo;
or one R¹ and one R⁵ are taken together to form a heterocycloalkyl optionally substituted with one, two, or three R¹⁻⁵;
each R¹⁻⁵ is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R¹⁻⁵ on the same carbon are taken together to form an oxo;
each R⁵ is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally substituted with one, two, or three R^{5a};
or two R⁵ on adjacent atoms are taken together to form a cycloalkyl, heterocycloalkyl, aryl, or heteroaryl;
each optionally substituted with one, two, or three R^{b};
each R^{5a} is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{5a} on the same carbon are taken together to form an oxo;
each R^{b} is independently deuterium, halogen, —CN, —OH, —OW, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{b} on the same carbon are taken together to form an oxo;
m is 0-4;
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl);
wherein each alkyl, alkenyl, alkynyl, cycloalkyl, heterocycloalkyl, aryl, and heteroaryl is independently optionally substituted with one, two, or three deuterium, oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆deuteroalkyl, C₁-C₆haloalkyl, C₁-C₆hydroxyalkyl, or C₁-C₆aminoalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl);
wherein each alkyl, alkenyl, alkynyl, cycloalkyl, heterocycloalkyl, aryl, and heteroaryl is independently optionally substituted with one, two, or three deuterium, oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆deuteroalkyl, C₁-C₆hydroxyalkyl, or C₁-C₆aminoalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one, two, or three oxo, deuterium, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆deuteroalkyl, C₁-C₆hydroxyalkyl, or C₁-C₆aminoalkyl;
or R^{c} and R^{d} are taken together with the atom to which they are attached to form a heterocycloalkyl optionally substituted with one, two, or three oxo, deuterium, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆deuteroalkyl, C₁-C₆hydroxyalkyl, or C₁-C₆aminoalkyl;
   provided that the compound of Formula (II) is not

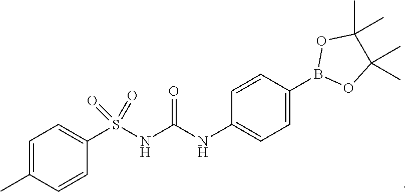

Also disclosed herein is a pharmaceutical composition comprising a compound disclosed herein, or a pharmaceutically acceptable salt, solvate, or stereoisomer thereof, and a pharmaceutically acceptable excipient.

Also disclosed herein is a method of treating of a disease which is responsive to inhibition of activation of the NLRP3 inflammasome in a subject in need thereof, the method comprising administering to the subject an effective amount of a compound disclosed herein, or a pharmaceutically acceptable salt, solvate, or stereoisomer thereof.

Also disclosed herein is a method of treating of a disease which is responsive to modulation of one or more of IL-6, IL-1 beta, IL-17, IL-18, IL-1 alpha, IL-37, TL-22, TL-33 and Th17 cells in a subject in need thereof, the method comprising administering to the subject an effective amount of a compound disclosed herein, or a pharmaceutically acceptable salt, solvate, or stereoisomer thereof. In some embodiments, the disease is an immune system disease, an inflammatory disease, an autoimmune disease, a skin disease, a cardiovascular disease, cancer, a renal system disease, a gastro-intestinal tract disease, a respiratory system disease, a disease of the endocrine system, a viral disease, or a central nervous system (CNS) disease.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“Oxo” refers to ═O.

“Carboxyl” refers to —COOH.

“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀ alkyl. In some embodiments, the alkyl is a C₁₋₆alkyl. In some embodiments, the alkyl is a C₁₋₅alkyl. In some embodiments, the alkyl is a C₁₋₄alkyl. In some embodiments, the alkyl is a C₁₋₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COOH, COOMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —COOH, COOMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula —OR^{a} where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, COOMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from a hydrocarbon ring system comprising 6 to 30 carbon atoms and at least one aromatic ring. The aryl radical may be a monocyclic, bicyclic, tricyclic or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Aryl radicals include, but are not limited to 1,2,3,5,6,7-hexahydro-s-indacene, 2,3-dihydro-1H-indene, 1,2,3,4-tetrahydronaphthalene, 2,3,5,6,7,8-hexahydro-1H-cyclopenta[b]naphthalene, and 1,2,3,4,5,6,7,8-octahydroanthracene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, COOMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Cycloalkyl” refers to a partially or fully saturated, monocyclic or polycyclic carbocyclic ring, which may include fused (when fused with an aryl or a heteroaryl ring, the cycloalkyl is bonded through a non-aromatic ring atom) or bridged ring systems. In some embodiments, the cycloalkyl is fully saturated. Representative cycloalkyls include, but are not limited to, cycloalkyls having from three to fifteen carbon atoms (C₃-C₁₅ cycloalkyl or C₃-C₁₅ cycloalkenyl), from three to ten carbon atoms (C₃-C₁₀ cycloalkyl or C₃-C₁₀ cycloalkenyl), from three to eight carbon atoms (C₃-C₈ cycloalkyl or C₃-C₈ cycloalkenyl), from three to six carbon atoms (C₃-C₆ cycloalkyl or C₃-C₆ cycloalkenyl), from three to five carbon atoms (C₃-C₈ cycloalkyl or C₃-C₈ cycloalkenyl), or three to four carbon atoms (C₃-C₄ cycloalkyl or C₃-C₄ cycloalkenyl). In some embodiments, the cycloalkyl is a 3- to 10-membered cycloalkyl or a 3- to 10-membered cycloalkenyl. In some embodiments, the cycloalkyl is a 3- to 6-membered cycloalkyl or a 3- to 6-membered cycloalkenyl. In some embodiments, the cycloalkyl is a 5- to 6-membered cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, COOMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Deuteroalkyl” refers to an alkyl radical, as defined above, that is substituted by one or more deuteriums. In some embodiments, the alkyl is substituted with one deuterium. In some embodiments, the alkyl is substituted with one, two, or three deuteriums. In some embodiments, the alkyl is substituted with one, two, three, four, five, or six deuteriums. Deuteroalkyl include, for example, CD₃, CH₂D, CHD₂, CH₂CD₃, CD₂CD₃, CHDCD₃, CH₂CH₂D, or CH₂CHD₂. In some embodiments, the deuteroalkyl is CD₃.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic or tetracyclic ring system, which may include fused (when fused with an aryl or a heteroaryl ring, the heterocycloalkyl is bonded through a non-aromatic ring atom) or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (C₂-C₁₅ heterocycloalkyl or C₂-C₁₅ heterocycloalkenyl), from two to ten carbon atoms (C₂-C₁₀ heterocycloalkyl or C₂-C₁₀ heterocycloalkenyl), from two to eight carbon atoms (C₂-C₈ heterocycloalkyl or C₂-C₈ heterocycloalkenyl), from two to seven carbon atoms (C₂-C₇ heterocycloalkyl or C₂-C₇ heterocycloalkenyl), from two to six carbon atoms (C₂-C₆ heterocycloalkyl or C₂-C₆ heterocycloalkenyl), from two to five carbon atoms (C₂-C₈ heterocycloalkyl or C₂-C₈ heterocycloalkenyl), or two to four carbon atoms (C₂-C₄ heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered heterocycloalkyl or a 3- to 8-membered heterocycloalkenyl. In some embodiments, the heterocycloalkyl is a 3- to 7-membered heterocycloalkyl or a 3- to 7-membered heterocycloalkenyl. In some embodiments, the heterocycloalkyl is a 3- to 6-membered heterocycloalkyl or a 3- to 6-membered heterocycloalkenyl. In some embodiments, the heterocycloalkyl is a 4- to 6-membered heterocycloalkyl or a 4- to 6-membered heterocycloalkenyl. In some embodiments, the heterocycloalkyl is a 5- to 6-membered heterocycloalkyl or a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —COOH, COOMe, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic or tetracyclic ring system, which may include fused (when fused with a cycloalkyl or heterocycloalkyl ring, the heteroaryl is bonded through an aromatic ring atom) or bridged ring systems; and the nitrogen, carbon or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically, up to about 500 daltons.

An “effective amount” or “therapeutically effective amount” refers to an amount of a compound administered to a mammalian subject, either as a single dose or as part of a series of doses, which is effective to produce a desired therapeutic effect.

“Treatment” of an individual (e.g. a mammal, such as a human) or a cell is any type of intervention used in an attempt to alter the natural course of the individual or cell. In some embodiments, treatment includes administration of a pharmaceutical composition, subsequent to the initiation of a pathologic event or contact with an etiologic agent and includes stabilization of the condition (e.g., condition does not worsen) or alleviation of the condition. In some embodiments, treatment also includes prophylactic treatment (e.g., administration of a composition described herein when an individual is suspected to be suffering from a disease).

“Synergy” or “synergize” refers to an effect of a combination that is greater than additive of the effects of each component alone at the same doses.

### Compounds

Disclosed herein are methods comprising administering an NLRP3 inflammasome inhibitor to a subject exhibiting increased levels of NLRP3 inflammasome activity or determined to be at risk for developing increased levels of NLRP3 inflammasome activity. Also disclosed herein are methods comprising administering an NLRP3 inflammasome inhibitor to a subject that has been diagnosed with an NLRP3 inflammasome-related disease or disorder, or who has symptoms or signs of an NLRP3 inflammasome-related disease or disorder.

Disclosed herein is a compound of Formula (I), or a pharmaceutically acceptable salt, solvate, or stereoisomer thereof:

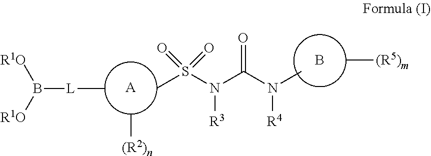

wherein:
Ring A is cycloalkyl, heterocycloalkyl, aryl, or heteroaryl;
L is a bond or a C₁-C₆ alkylene optionally substituted with one, two, or three R^{L};
each R^{L} is independently deuterium, halogen, —CN, —OH, —OW, —SH, —SW, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{L} on the same carbon are taken together to form an oxo;
each R¹ is independently hydrogen or C₁-C₆ alkyl;
or two R¹ are taken together to form a heterocycloalkyl optionally substituted with one, two, three, four, or five R^{1a};
each R^{1a} is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₁-C₆alkylene(COOH), C₂-C₆alkenyl, C₂-C₆alkynyl, cycloalkyl, heterocycloalkyl, aryl, or heteroaryl;
or two R^{1a} on the same carbon are taken together to form an oxo;
each R² is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally substituted with one, two, or three R^{2a};
each R^{2a} is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{2a} on the same carbon are taken together to form an oxo;
n is 0-3;
or one R¹ and one R² are taken together to form a heterocycloalkyl optionally substituted with one, two, or three R¹⁻²;
each R¹⁻² is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R¹⁻² on the same carbon are taken together to form an oxo;
R³ and R⁴ are each independently hydrogen or C₁-C₆ alkyl;
Ring B is cycloalkyl, heterocycloalkyl, aryl, or heteroaryl;
each R⁵ is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally substituted with one, two, or three R^{5a};
or two R⁵ on adjacent atoms are taken together to form a cycloalkyl, heterocycloalkyl, aryl, or heteroaryl;
each optionally substituted with one, two, or three R^{b};
each R^{5a} is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{5a} on the same carbon are taken together to form an oxo;
each R^{b} is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{b} on the same carbon are taken together to form an oxo;
m is 0-5;
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl);
wherein each alkyl, alkenyl, alkynyl, cycloalkyl, heterocycloalkyl, aryl, and heteroaryl is independently optionally substituted with one, two, or three deuterium, oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆deuteroalkyl, C₁-C₆haloalkyl, C₁-C₆hydroxyalkyl, or C₁-C₆aminoalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl);
wherein each alkyl, alkenyl, alkynyl, cycloalkyl, heterocycloalkyl, aryl, and heteroaryl is independently optionally substituted with one, two, or three deuterium, oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆deuteroalkyl, C₁-C₆hydroxyalkyl, or C₁-C₆aminoalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one, two, or three oxo, deuterium, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆deuteroalkyl, C₁-C₆hydroxyalkyl, or C₁-C₆aminoalkyl;
or R^{c} and R^{d} are taken together with the atom to which they are attached to form a heterocycloalkyl optionally substituted with one, two, or three oxo, deuterium, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆deuteroalkyl, C₁-C₆hydroxyalkyl, or C₁-C₆aminoalkyl.

In some embodiments of a compound of Formula (I), Ring A is phenyl or a 5- or 6-membered heteroaryl. In some embodiments of a compound of Formula (I), Ring A is phenyl. In some embodiments of a compound of Formula (I), Ring A is pyridinyl. In some embodiments of a compound of Formula (I), Ring A is a 5-membered heteroaryl. In some embodiments of a compound of Formula (I), Ring A is cycloalkyl or heterocycloalkyl.

In some embodiments of a compound of Formula (I), each R¹ is hydrogen or two R¹ are taken together to form a heterocycloalkyl optionally substituted with one, two, three, four, or five R^{1a}. In some embodiments of a compound of Formula (I), each R¹ is hydrogen. In some embodiments of a compound of Formula (I), two R¹ are taken together to form a heterocycloalkyl optionally substituted with one, two, three, four, or five R^{1a}.

In some embodiments of a compound of Formula (I), two R¹ are taken together to form

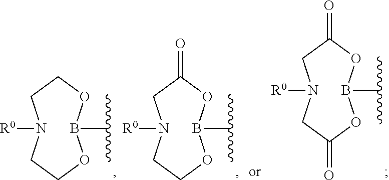

wherein R⁰ is hydrogen, C₁-C₆alkyl, C₁-C₆hydroxyalkyl, or C₁-C₆alkylene(COOH).

In some embodiments of a compound of Formula (I), R⁰ is hydrogen or C₁-C₆alkyl. In some embodiments of a compound of Formula (I), R⁰ is C₁-C₆alkyl.

In some embodiments of a compound of Formula (I), two R¹ are taken together to form

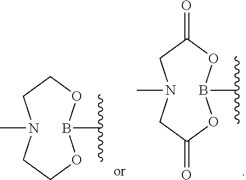

In some embodiments of a compound of Formula (I), each R^{1a} is independently deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; or two R^{1a} on the same carbon are taken together to form an oxo. In some embodiments of a compound of Formula (I), each R^{1a} is independently deuterium, halogen, —CN, —OH, —OR^{a}, —NR^{c}R^{d}, C₁-C₆alkyl, C₁-C₆haloalkyl, or C₁-C₆deuteroalkyl; or two R^{1a} on the same carbon are taken together to form an oxo. In some embodiments of a compound of Formula (I), each R^{1a} is independently deuterium, halogen, C₁-C₆alkyl, C₁-C₆haloalkyl, or C₁-C₆deuteroalkyl; or two R^{1a} on the same carbon are taken together to form an oxo. In some embodiments of a compound of Formula (I), each R^{1a} is independently C₁-C₆alkyl.

In some embodiments of a compound of Formula (I), L is C₁-C₂ alkylene. In some embodiments of a compound of Formula (I), L is C₁ alkylene. In some embodiments of a compound of Formula (I), L is C₂ alkylene. In some embodiments of a compound of Formula (I), L is a bond.

In some embodiments of a compound of Formula (I), each R² is independently deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is optionally substituted with one, two, or three R^{2a}. In some embodiments of a compound of Formula (I), each R² is independently deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, or C₁-C₆aminoalkyl; wherein each alkyl is optionally substituted with one, two, or three R^{2a}. In some embodiments of a compound of Formula (I), each R² is independently halogen, —CN, —OH, —OW, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (I), each R² is independently halogen, —CN, —OH, —OR^{a}, —NR^{c}R^{d}, C₁-C₆alkyl, C₁-C₆haloalkyl, C₁-C₆deuteroalkyl, cycloalkyl, or heterocycloalkyl. In some embodiments of a compound of Formula (I), each R² is independently halogen, C₁-C₆alkyl, or cycloalkyl.

In some embodiments of a compound of Formula (I), each R^{2a} is independently deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; or two R^{2a} on the same carbon are taken together to form an oxo. In some embodiments of a compound of Formula (I), each R^{2a} is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, or C₁-C₆aminoalkyl; or two R^{2a} on the same carbon are taken together to form an oxo.

In some embodiments of a compound of Formula (I), n is 0-2. In some embodiments of a compound of Formula (I), n is 0 or 1. In some embodiments of a compound of Formula (I), n is 0. In some embodiments of a compound of Formula (I), n is 1. In some embodiments of a compound of Formula (I), n is 2. In some embodiments of a compound of Formula (I), n is 3.

In some embodiments of a compound of Formula (I), one R¹ and one Rare taken together to form a heterocycloalkyl optionally substituted with one, two, or three R¹⁻². In some embodiments of a compound of Formula (I), one R¹ and one R² are taken together to form a 5- or 6-membered heterocycloalkyl optionally substituted with one, two, or three R¹⁻². In some embodiments of a compound of Formula (I), one R¹ and one Rare taken together to form a 5- or 6-membered heterocycloalkyl optionally substituted with one, two, or three R¹⁻²; wherein the 5- or 6-membered heterocycloalkyl is 1,2-oxaborolane or 1,2-oxaborinane.

In some embodiments of a compound of Formula (I), each R¹⁻² is independently deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; or two R¹⁻² on the same carbon are taken together to form an oxo. In some embodiments of a compound of Formula (I), each R¹⁻² is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, or C₁-C₆aminoalkyl; or two R¹⁻² on the same carbon are taken together to form an oxo. In some embodiments of a compound of Formula (I), each R¹⁻² is independently C₁-C₆alkyl.

In some embodiments of a compound of Formula (I),

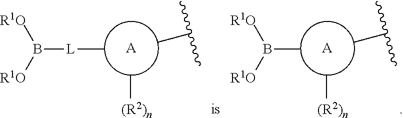

In some embodiments of a compound of Formula (I),

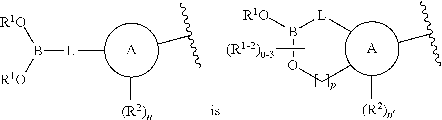

wherein p is 1-3 and n′ is 0-2. In some embodiments of a compound of Formula (I),

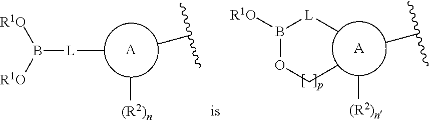

wherein p is 1-3 and n′ is 0-2.

In some embodiments of a compound of Formula (I), p is 1 or 2. In some embodiments of a compound of Formula (I), p is 1. In some embodiments of a compound of Formula (I), p is 2. 1000511 In some embodiments of a compound of Formula (I), n′ is 0 or 1. In some embodiments of a compound of Formula (I), n′ is 0. In some embodiments of a compound of Formula (I), n′ is 1. In some embodiments of a compound of Formula (I), n′ is 2.

In some embodiments of a compound of Formula (I),

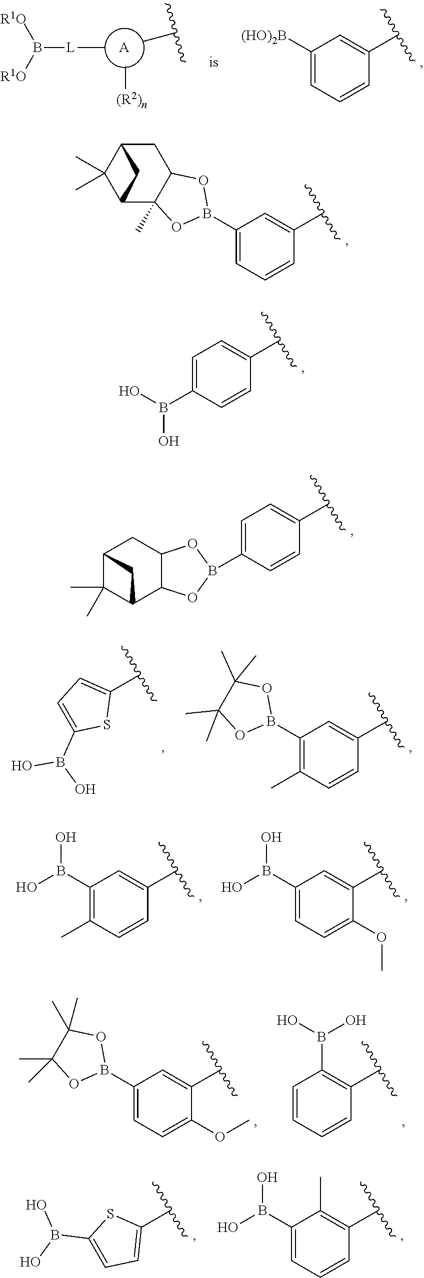

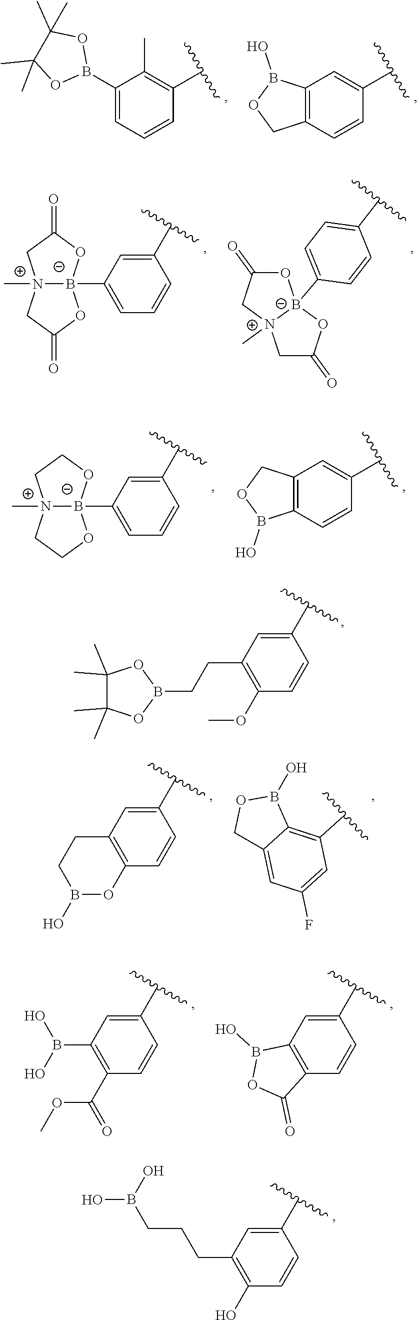

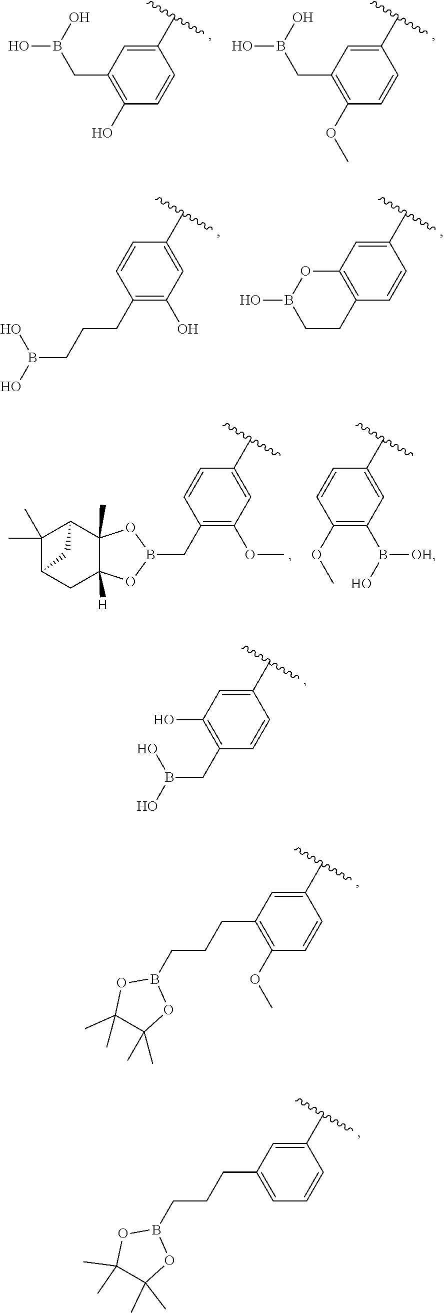

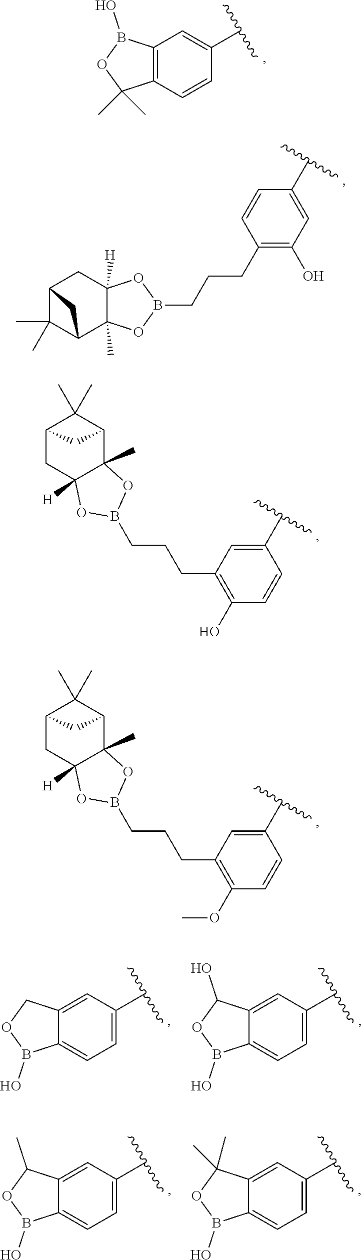

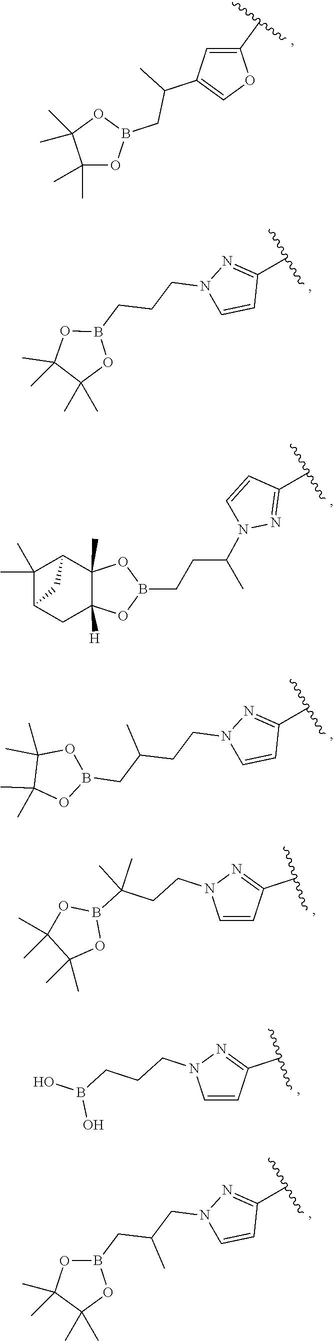

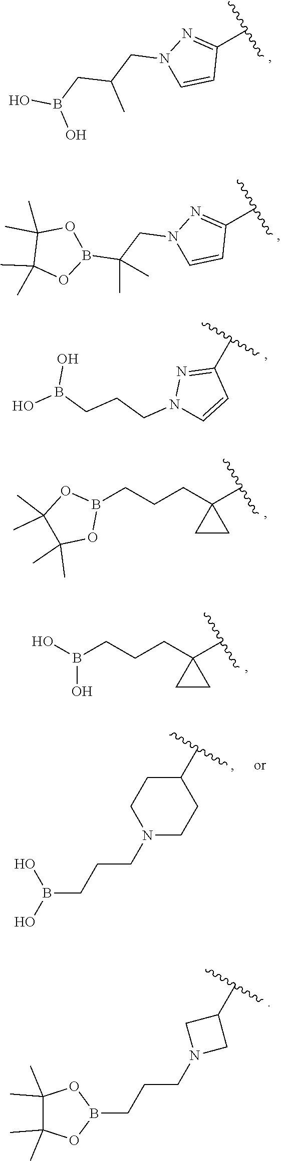

In some embodiments of a compound of Formula (I), R³ is hydrogen or methyl. In some embodiments of a compound of Formula (I), R³ is hydrogen.

In some embodiments of a compound of Formula (I), R⁴ is hydrogen or methyl. In some embodiments of a compound of Formula (I), R⁴ is hydrogen.

In some embodiments of a compound of Formula (I), Ring B is cycloalkyl or heterocycloalkyl. In some embodiments of a compound of Formula (I), Ring B is aryl or heteroaryl. In some embodiments of a compound of Formula (I), Ring B is aryl. In some embodiments of a compound of Formula (I), Ring B is phenyl.

In some embodiments of a compound of Formula (I), each R⁵ is independently deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is optionally substituted with one, two, or three R^{a}; or two R⁵ on adjacent atoms are taken together to form a cycloalkyl, heterocycloalkyl, aryl, or heteroaryl; each optionally substituted with one, two, or three R^{5b}. In some embodiments of a compound of Formula (I), each R⁵ is independently deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is optionally substituted with one, two, or three R^{5a}; or two R⁵ on adjacent atoms are taken together to form a cycloalkyl optionally substituted with one, two, or three R^{5b}.

In some embodiments of a compound of Formula (I), each R⁵ is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl; wherein each alkyl, cycloalkyl, and heterocycloalkyl is optionally substituted with one, two, or three R^{5a}; or two R⁵ on adjacent atoms are taken together to form a cycloalkyl optionally substituted with one, two, or three R^{5b}. In some embodiments of a compound of Formula (I), each R⁵ is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is optionally substituted with one, two, or three R^{5a}. In some embodiments of a compound of Formula (I), each R⁵ is independently halogen, —CN, —OH, —OR^{a}, —NR^{c}R^{d}, C₁-C₆alkyl, C₁-C₆haloalkyl, C₁-C₆deuteroalkyl, cycloalkyl, or heterocycloalkyl; wherein each alkyl, cycloalkyl, and heterocycloalkyl is optionally substituted with one, two, or three R^{5a}. In some embodiments of a compound of Formula (I), each R⁵ is independently halogen or C₁-C₆alkyl. In some embodiments of a compound of Formula (I), each R⁵ is independently C₁-C₆alkyl. In some embodiments of a compound of Formula (I), each R⁵ is independently C₁-C₆alkyl, cycloalkyl, or heteroaryl. In some embodiments of a compound of Formula (I), each R⁵ is independently halogen, C₁-C₆alkyl, or heteroaryl; wherein each alkyl heteroaryl is optionally substituted with one, two, or three R^{5a}.

In some embodiments of a compound of Formula (I), two R⁵ on adjacent atoms are taken together to form a cycloalkyl optionally substituted with one, two, or three R^{5b}.

In some embodiments of a compound of Formula (I), each R^{5b} is independently deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; or two R^{5b} on the same carbon are taken together to form an oxo. In some embodiments of a compound of Formula (I), each R^{5b} is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, or C₁-C₆aminoalkyl; or two R^{5b} on the same carbon are taken together to form an oxo.

In some embodiments of a compound of Formula (I), m is 0-4. In some embodiments of a compound of Formula (I), m is 0-3. In some embodiments of a compound of Formula (I), m is 0-2. In some embodiments of a compound of Formula (I), m is 0 or 1. In some embodiments of a compound of Formula (I), m is 2-4. In some embodiments of a compound of Formula (I), m is 2-5. In some embodiments of a compound of Formula (I), m is 3-5. In some embodiments of a compound of Formula (I), m is 0. In some embodiments of a compound of Formula (I), m is 1. In some embodiments of a compound of Formula (I), m is 2. In some embodiments of a compound of Formula (I), m is 3. In some embodiments of a compound of Formula (I), m is 4.

In some embodiments of a compound of Formula (I),

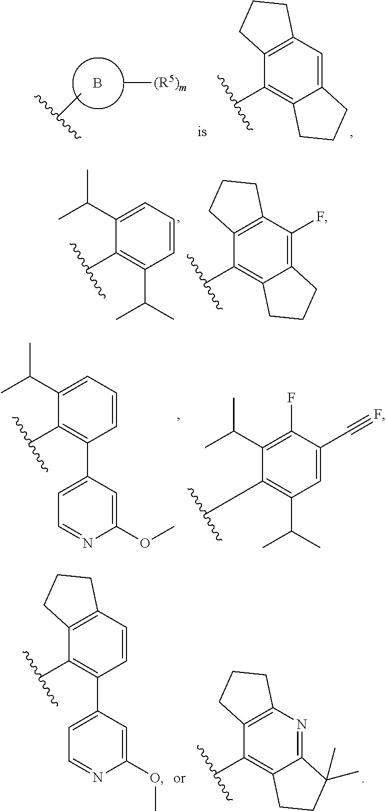

In some embodiments of a compound of Formula (I),

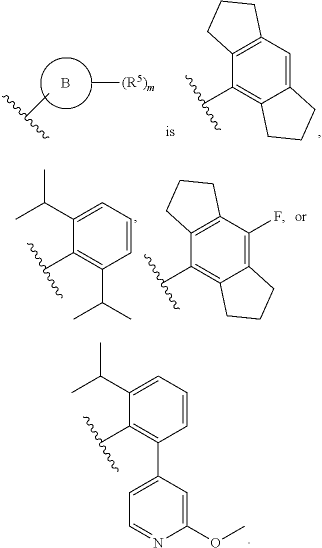

In some embodiments of a compound of Formula (I),

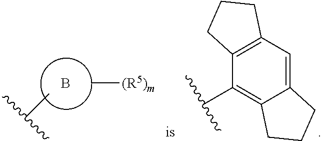

In some embodiments of a compound of Formula (I),

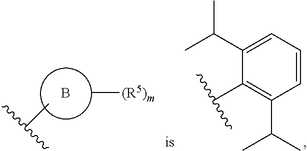

Also disclosed herein is a compound of Formula (II), or a pharmaceutically acceptable salt, solvate, or stereoisomer thereof:

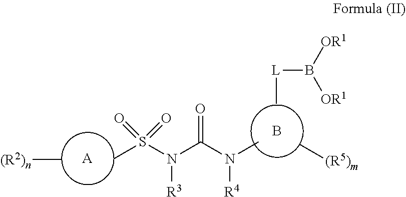

wherein:
Ring A is cycloalkyl, heterocycloalkyl, aryl, or heteroaryl;
each R² is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally substituted with one, two, or three R^{2a};
each R^{2a} is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{2a} on the same carbon are taken together to form an oxo;
n is 0-3;
R³ and R⁴ are each independently hydrogen or C₁-C₆ alkyl;
Ring B is cycloalkyl, heterocycloalkyl, aryl, or heteroaryl;
L is a bond or a C₁-C₆ alkylene optionally substituted with one, two, or three R^{L};
each R^{L} is independently deuterium, halogen, —CN, —OH, —OW, —SH, —SW, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{L} on the same carbon are taken together to form an oxo;
each R¹ is independently hydrogen or C₁-C₆ alkyl;
or two R¹ are taken together to form a heterocycloalkyl optionally substituted with one, two, three, four, or five R^{1a};
each R^{1a} is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₁-C₆alkylene(COOH), C₂-C₆alkenyl, C₂-C₆alkynyl, cycloalkyl, heterocycloalkyl, aryl, or heteroaryl;
or two R^{1a} on the same carbon are taken together to form an oxo;
or one R¹ and one R⁵ are taken together to form a heterocycloalkyl optionally substituted with one, two, or three R¹⁻⁵;
each R¹⁻⁵ is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R¹⁻⁵ on the same carbon are taken together to form an oxo;
each R⁵ is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally substituted with one, two, or three R^{5a};
or two R⁵ on adjacent atoms are taken together to form a cycloalkyl, heterocycloalkyl, aryl, or heteroaryl;
each optionally substituted with one, two, or three R^{b};
each R^{5a} is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{5a} on the same carbon are taken together to form an oxo;
each R^{b} is independently deuterium, halogen, —CN, —OH, —OW, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{b} on the same carbon are taken together to form an oxo;
m is 0-4;
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl);
wherein each alkyl, alkenyl, alkynyl, cycloalkyl, heterocycloalkyl, aryl, and heteroaryl is independently optionally substituted with one, two, or three deuterium, oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆deuteroalkyl, C₁-C₆haloalkyl, C₁-C₆hydroxyalkyl, or C₁-C₆aminoalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl);
wherein each alkyl, alkenyl, alkynyl, cycloalkyl, heterocycloalkyl, aryl, and heteroaryl is independently optionally substituted with one, two, or three deuterium, oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆deuteroalkyl, C₁-C₆hydroxyalkyl, or C₁-C₆aminoalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one, two, or three oxo, deuterium, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆deuteroalkyl, C₁-C₆hydroxyalkyl, or C₁-C₆aminoalkyl;
or R^{c} and R^{d} are taken together with the atom to which they are attached to form a heterocycloalkyl optionally substituted with one, two, or three oxo, deuterium, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆deuteroalkyl, C₁-C₆hydroxyalkyl, or C₁-C₆aminoalkyl;
provided that the compound of Formula (II) is not

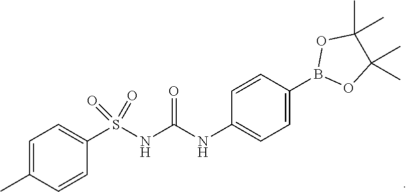

In some embodiments of a compound of Formula (II), Ring A is phenyl or a 5- or 6-membered heteroaryl. In some embodiments of a compound of Formula (II), Ring A is phenyl. In some embodiments of a compound of Formula (II), Ring A is pyridinyl. In some embodiments of a compound of Formula (II), Ring A is a 5-membered heteroaryl. In some embodiments of a compound of Formula (II), Ring A is cycloalkyl or heterocycloalkyl.

In some embodiments of a compound of Formula (II), each R² is independently deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is optionally substituted with one, two, or three R^{2a}. In some embodiments of a compound of Formula (II), each R² is independently deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, or C₁-C₆aminoalkyl; wherein each alkyl is optionally substituted with one, two, or three R^{2a}. In some embodiments of a compound of Formula (II), each R² is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (II), each R² is independently halogen, —CN, —OH, —OR^{a}, —NR^{c}R^{d}, C₁-C₆alkyl, C₁-C₆haloalkyl, C₁-C₆deuteroalkyl, cycloalkyl, or heterocycloalkyl. In some embodiments of a compound of Formula (II), each R² is independently halogen, C₁-C₆alkyl, or cycloalkyl.

In some embodiments of a compound of Formula (II), each R^{2a} is independently deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; or two R^{2a} on the same carbon are taken together to form an oxo. In some embodiments of a compound of Formula (II), each R^{2a} is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, or C₁-C₆aminoalkyl; or two R^{2a} on the same carbon are taken together to form an oxo.

In some embodiments of a compound of Formula (II), n is 0-2. In some embodiments of a compound of Formula (II), n is 0 or 1. In some embodiments of a compound of Formula (II), n is 0. In some embodiments of a compound of Formula (II), n is 1. In some embodiments of a compound of Formula (II), n is 2. In some embodiments of a compound of Formula (II), n is 3.

In some embodiments of a compound of Formula (II),

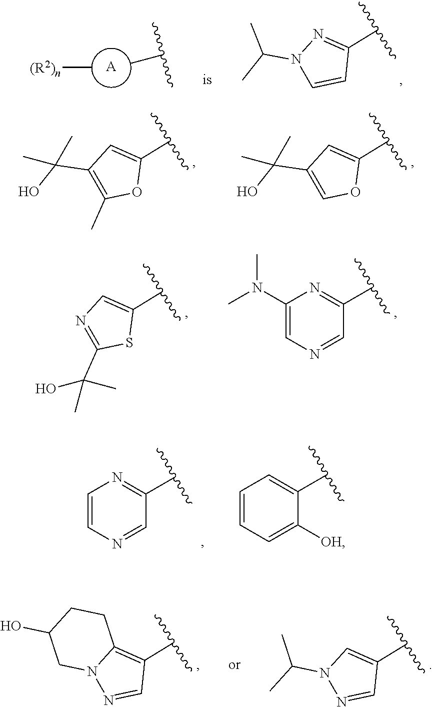

In some embodiments of a compound of Formula (II)

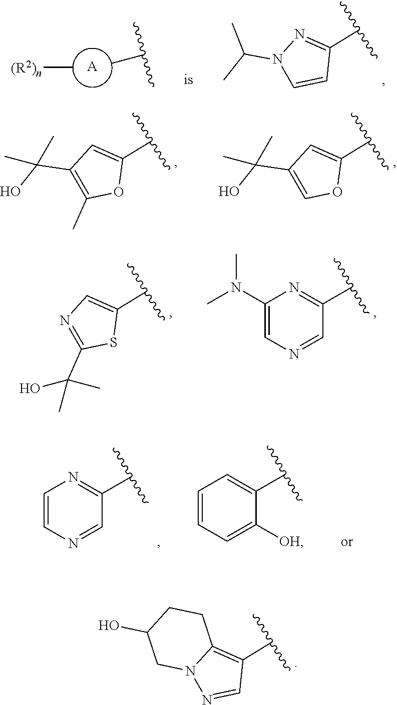

In some embodiments of a compound of Formula (II),

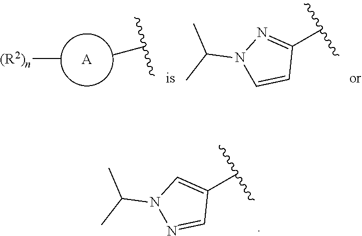

In some embodiments of a compound of Formula (II), R³ is hydrogen or methyl. In some embodiments of a compound of Formula (II), R³ is hydrogen.

In some embodiments of a compound of Formula (II), R⁴ is hydrogen or methyl. In some embodiments of a compound of Formula (II), R⁴ is hydrogen.

In some embodiments of a compound of Formula (II), Ring B is cycloalkyl or heterocycloalkyl. In some embodiments of a compound of Formula (II), Ring B is aryl or heteroaryl. In some embodiments of a compound of Formula (II), Ring B is aryl. In some embodiments of a compound of Formula (II), Ring B is phenyl.

In some embodiments of a compound of Formula (II), each R¹ is hydrogen or two R¹ are taken together to form a heterocycloalkyl optionally substituted with one, two, three, four, or five R^{1a}. In some embodiments of a compound of Formula (II), each R¹ is hydrogen. In some embodiments of a compound of Formula (II), two R¹ are taken together to form a heterocycloalkyl optionally substituted with one, two, three, four, or five R^{1a}.

In some embodiments of a compound of Formula (II), two R¹ are taken together to form

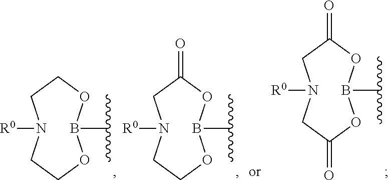

wherein R⁰ is hydrogen, C₁-C₆alkyl, C₁-C₆hydroxyalkyl, or C₁-C₆alkylene(COOH).

In some embodiments of a compound of Formula (I), R⁰ is hydrogen or C₁-C₆alkyl. In some embodiments of a compound of Formula (I), R⁰ is C₁-C₆alkyl.

In some embodiments of a compound of Formula (II), two R¹ are taken together to form

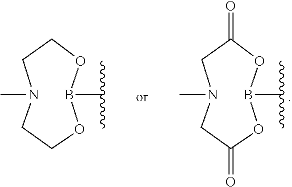

In some embodiments of a compound of Formula (II), each R^{1a} is independently deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; or two R^{1a} on the same carbon are taken together to form an oxo. In some embodiments of a compound of Formula (II), each R^{1a} is independently deuterium, halogen, —CN, —OH, —OW, —NR^{c}R^{d}, C₁-C₆alkyl, C₁-C₆haloalkyl, or C₁-C₆deuteroalkyl; or two R^{1a} on the same carbon are taken together to form an oxo. In some embodiments of a compound of Formula (II), each R^{1a} is independently deuterium, halogen, C₁-C₆alkyl, C₁-C₆haloalkyl, or C₁-C₆deuteroalkyl; or two R^{1a} on the same carbon are taken together to form an oxo. In some embodiments of a compound of Formula (II), each R^{1a} is independently C₁-C₆alkyl.

In some embodiments of a compound of Formula (II), L is C₁-C₂ alkylene. In some embodiments of a compound of Formula (II), L is C₁ alkylene. In some embodiments of a compound of Formula (II), L is C₂ alkylene. In some embodiments of a compound of Formula (II), L is a bond.

In some embodiments of a compound of Formula (II), each R⁵ is independently deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is optionally substituted with one, two, or three R^{a}; or two R⁵ on adjacent atoms are taken together to form a cycloalkyl, heterocycloalkyl, aryl, or heteroaryl; each optionally substituted with one, two, or three R^{5b}. In some embodiments of a compound of Formula (II), each R⁵ is independently deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is optionally substituted with one, two, or three R^{5a}; or two R⁵ on adjacent atoms are taken together to form a cycloalkyl optionally substituted with one, two, or three R^{5b}.

In some embodiments of a compound of Formula (II), each R⁵ is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl; wherein each alkyl, cycloalkyl, and heterocycloalkyl is optionally substituted with one, two, or three R^{5a}; or two R⁵ on adjacent atoms are taken together to form a cycloalkyl optionally substituted with one, two, or three R^{5b}. In some embodiments of a compound of Formula (II), each R⁵ is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is optionally substituted with one, two, or three R^{5a}. In some embodiments of a compound of Formula (II), each R⁵ is independently halogen, —CN, —OH, —OW, —NR^{c}R^{d}, C₁-C₆alkyl, C₁-C₆haloalkyl, C₁-C₆deuteroalkyl, cycloalkyl, or heterocycloalkyl; wherein each alkyl, cycloalkyl, and heterocycloalkyl is optionally substituted with one, two, or three R^{5a}. In some embodiments of a compound of Formula (II), each R⁵ is independently halogen or C₁-C₆alkyl. In some embodiments of a compound of Formula (II), each R⁵ is independently C₁-C₆alkyl. In some embodiments of a compound of Formula (II), each R⁵ is independently C₁-C₆alkyl, cycloalkyl, or heteroaryl. In some embodiments of a compound of Formula (II), each R⁵ is independently halogen, C₁-C₆alkyl, or heteroaryl; wherein each alkyl heteroaryl is optionally substituted with one, two, or three R^{5a}.

In some embodiments of a compound of Formula (II), two R⁵ on adjacent atoms are taken together to form a cycloalkyl optionally substituted with one, two, or three R^{5b}.

In some embodiments of a compound of Formula (II), each R^{5b} is independently deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; or two R^{5b} on the same carbon are taken together to form an oxo. In some embodiments of a compound of Formula (II), each R^{5b} is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, or C₁-C₆aminoalkyl; or two R^{5b} on the same carbon are taken together to form an oxo.

In some embodiments of a compound of Formula (II), m is 0-3. In some embodiments of a compound of Formula (II), m is 0-2. In some embodiments of a compound of Formula (II), m is 0 or 1. In some embodiments of a compound of Formula (II), m is 2-4. In some embodiments of a compound of Formula (II), m is 2-5. In some embodiments of a compound of Formula (II), m is 3-5. In some embodiments of a compound of Formula (II), m is 0. In some embodiments of a compound of Formula (II), m is 1. In some embodiments of a compound of Formula (II), m is 2. In some embodiments of a compound of Formula (II), m is 3. In some embodiments of a compound of Formula (II), m is 4.

In some embodiments of a compound of Formula (II), one R¹ and one R⁵ are taken together to form a heterocycloalkyl optionally substituted with one, two, or three R¹⁻⁵. In some embodiments of a compound of Formula (II), one R¹ and one R⁵ are taken together to form a 5- or 6-membered heterocycloalkyl optionally substituted with one, two, or three R¹⁻⁵. In some embodiments of a compound of Formula (II), one R¹ and one R⁵ are taken together to form a 5- or 6-membered heterocycloalkyl optionally substituted with one, two, or three R¹⁻⁵; wherein the 5- or 6-membered heterocycloalkyl is 1,2-oxaborolane or 1,2-oxaborinane.

In some embodiments of a compound of Formula (II), each R¹⁻⁵ is independently deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; or two R¹⁻⁵ on the same carbon are taken together to form an oxo. In some embodiments of a compound of Formula (II), each R¹⁻⁵ is independently deuterium, halogen, —CN, —OH, —OW, —NR^{c}R^{d}, C₁-C₆alkyl, C₁-C₆haloalkyl, C₁-C₆deuteroalkyl, C₁-C₆hydroxyalkyl, or C₁-C₆aminoalkyl; or two R¹⁻⁵ on the same carbon are taken together to form an oxo. In some embodiments of a compound of Formula (II), each R¹⁻⁵ is independently C₁-C₆alkyl.

In some embodiments of a compound of Formula (II),

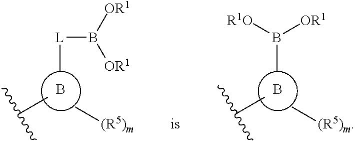

In some embodiments of a compound of Formula (II),

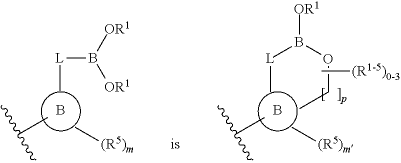

wherein p is 1-3 and m′ is 0-3. In some embodiments of a compound of Formula (II),

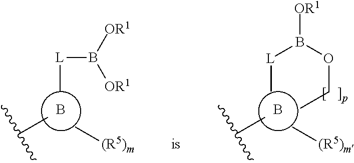

wherein p is 1-3 and m′ is 0-3.

In some embodiments of a compound of Formula (II), p is 1 or 2. In some embodiments of a compound of Formula (II), p is 1. In some embodiments of a compound of Formula (II), p is 2.

In some embodiments of a compound of Formula (II), m′ is 0 or 1. In some embodiments of a compound of Formula (II), m′ is 0. In some embodiments of a compound of Formula (II), m′ is 1. In some embodiments of a compound of Formula (II), m′ is 2.

In some embodiments of a compound of Formula (II),

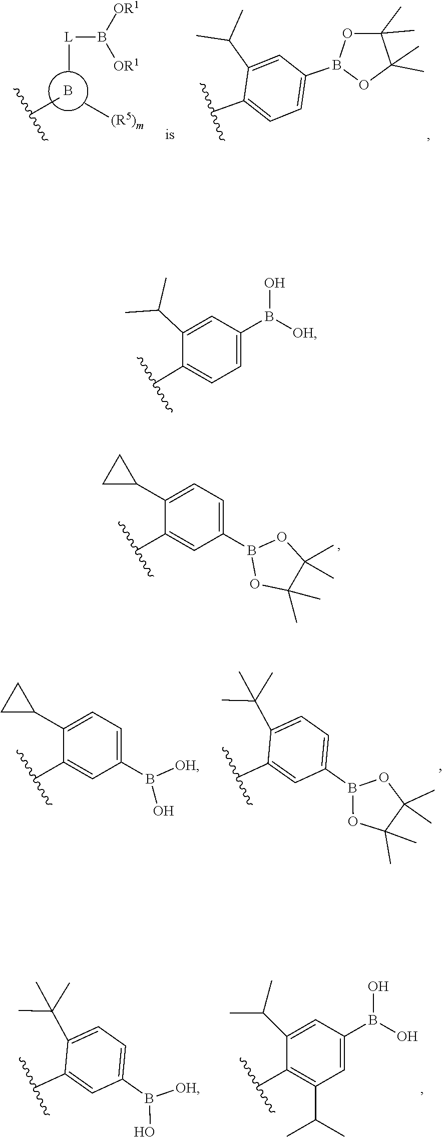

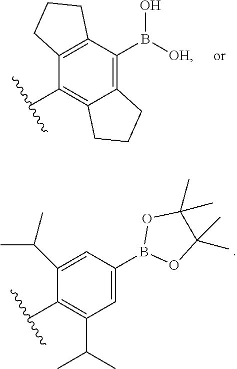 [SMILES: *c1c2c(c(B(O)O)c3c1CCC3)CCC2 ; *c1c(C(C)C)cc(B2OC(C)(C)C(C)(C)O2)cc1C(C)C]

In some embodiments of a compound of Formula (I) or (II), each R^{a} is independently C₁-C₆alkyl, C₁-C₆haloalkyl, C₁-C₆hydroxyalkyl, C₁-C₆aminoalkyl, cycloalkyl, heterocycloalkyl, aryl, or heteroaryl. In some embodiments of a compound of Formula (I) or (II), each R^{a} is independently C₁-C₆alkyl, C₁-C₆haloalkyl, cycloalkyl, or heterocycloalkyl. In some embodiments of a compound of Formula (I) or (II), each R^{a} is independently C₁-C₆alkyl, C₁-C₆haloalkyl, or cycloalkyl. In some embodiments of a compound of Formula (I) or (II), each R^{a} is independently C₁-C₆alkyl or C₁-C₆haloalkyl. In some embodiments of a compound of Formula (I) or (II), each R^{a} is independently C₁-C₆alkyl.

In some embodiments of a compound of Formula (I) or (II), each R^{b} is independently hydrogen, C₁-C₆alkyl, C₁-C₆haloalkyl, C₁-C₆hydroxyalkyl, C₁-C₆aminoalkyl, cycloalkyl, heterocycloalkyl, aryl, or heteroaryl. In some embodiments of a compound of Formula (I) or (II), each R^{b} is independently hydrogen, C₁-C₆alkyl, C₁-C₆haloalkyl, cycloalkyl, or heterocycloalkyl. In some embodiments of a compound of Formula (I) or (II), each R^{b} is independently hydrogen, C₁-C₆alkyl, C₁-C₆haloalkyl, or cycloalkyl. In some embodiments of a compound of Formula (I) or (II), each R^{b} is independently hydrogen, C₁-C₆alkyl, or C₁-C₆haloalkyl. In some embodiments of a compound of Formula (I) or (II), each R^{b} is independently hydrogen or C₁-C₆alkyl. In some embodiments of a compound of Formula (I) or (II), each R^{b} is hydrogen. In some embodiments of a compound of Formula (I) or (II), each R^{b} is independently C₁-C₆alkyl.

In some embodiments of a compound of Formula (I) or (II), each R^{c} and R^{d} is independently hydrogen, C₁-C₆alkyl, C₁-C₆haloalkyl, C₁-C₆hydroxyalkyl, C₁-C₆aminoalkyl, cycloalkyl, heterocycloalkyl, aryl, or heteroaryl. In some embodiments of a compound of Formula (I) or (II), each R^{c} and R^{d} is independently hydrogen, C₁-C₆alkyl, C₁-C₆haloalkyl, cycloalkyl, or heterocycloalkyl. In some embodiments of a compound of Formula (I) or (II), each R^{c} and R^{d} is independently hydrogen, C₁-C₆alkyl, C₁-C₆haloalkyl, or cycloalkyl. In some embodiments of a compound of Formula (I) or (II), each R^{c} and R^{d} is independently hydrogen, C₁-C₆alkyl, or C₁-C₆haloalkyl. In some embodiments of a compound of Formula (I) or (II), each R^{c} and R^{d} is independently hydrogen or C₁-C₆alkyl. In some embodiments of a compound of Formula (I) or (II), each R^{c} and R^{d} is hydrogen.

In some embodiments of a compound of Formula (I) or (II), R^{c} and R^{d} are taken together with the atom to which they are attached to form a heterocycloalkyl optionally substituted with one, two, or three halogen, C₁-C₆alkyl, or C₁-C₆haloalkyl.

Any combination of the groups described above for the various variables is contemplated herein. Throughout the specification, groups and substituents thereof are chosen by one skilled in the field to provide stable moieties and compounds.

Described herein is a compound of Formula (I) or (II), or a pharmaceutically acceptable salt, solvate, or stereoisomer thereof, selected from a compound in Table 1.

TABLE 1
Example compounds.

| | | | ESI-MS |
|---|---|---|---|
| Ex. | Structure | Chemical Name | (M + H)⁺ (m/z) |
| 1 | 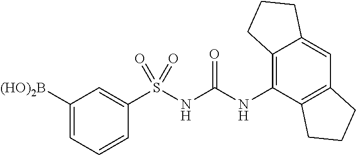 [SMILES: O=C(Nc1c2c(cc3c1CCC3)CCC2)NS(=O)(=O)c1cccc(B(O)O)c1] | (3-(N-((1,2,3,5,6,7-hexahydro-s- indacen-4- yl)carbamoyl)sulfamoyl)phenyl) boronic acid | 401 |
| 2 | 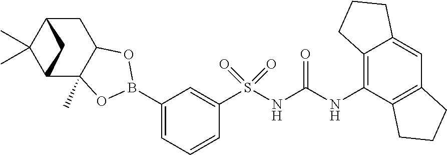 | N-((1,2,3,5,6,7-hexahydro-s-indacen-4- yl)carbamoyl)-3-((3aS,4R,6S)-3a,5,5- trimethylhexahydro-4,6- methanobenzo[d][1,3,2]dioxaborol-2- yl)benzenesulfonamide | 535 |
| 3 | 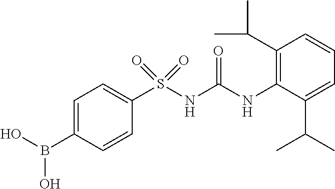 [SMILES: CC(C)c1cccc(C(C)C)c1NC(=O)NS(=O)(=O)c1ccc(B(O)O)cc1] | (4-(N-((2,6- diisopropylphenyl)carbamoyl) sulfamoyl)phenyl)boronic acid | 405 |
| 4 | 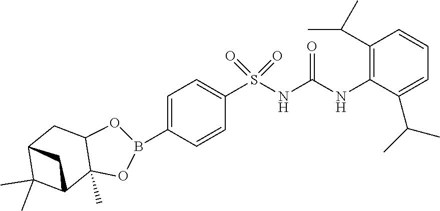 | N-((2,6-diisopropylphenyl)carbamoyl)- 4-((3aS,4R,6S)-3a,5,5- trimethylhexahydro-4,6- methanobenzo[d][1,3,2]dioxaborol-2- yl)benzenesulfonamide | 539 |
| 5 | 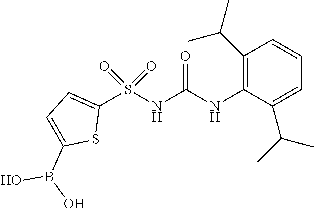 [SMILES: CC(C)c1cccc(C(C)C)c1NC(=O)NS(=O)(=O)c1ccc(B(O)O)s1] | (5-(N-((2,6-diisopropylphenyl) carbamoyl)sulfamoyl) thiophen-2-yl)boronic acid | 411 |
| 6 | 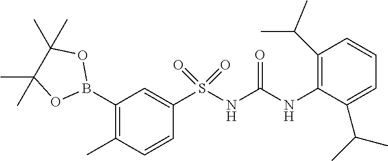 [SMILES: Cc1ccc(S(=O)(=O)NC(=O)Nc2c(C(C)C)cccc2C(C)C)cc1B1OC(C)(C)C(C)(C)O1] | N-((2,6-diisopropylphenyl)carbamoyl)- 4-methyl-3-(4,4,5,5-tetramethyl-1,3,2- dioxaborolan-2-yl)benzenesulfonamide | 501 |
| 7 | 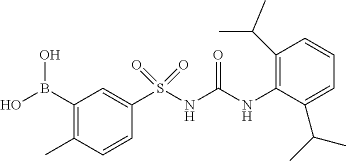 [SMILES: Cc1ccc(S(=O)(=O)NC(=O)Nc2c(C(C)C)cccc2C(C)C)cc1B(O)O] | (5-(N-((2,6-diisopropylphenyl) carbamoyl)sulfamoyl)- 2-methylphenyl)boronic acid | 419 |
| 8 | 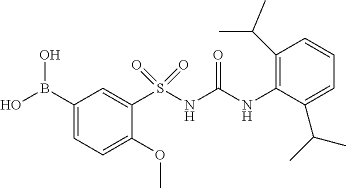 [SMILES: COc1ccc(B(O)O)cc1S(=O)(=O)NC(=O)Nc1c(C(C)C)cccc1C(C)C] | (3-(N-((2,6-diisopropylphenyl) carbamoyl)sulfamoyl)- 4-methoxyphenyl)boronic acid | 435 |
| 9 | 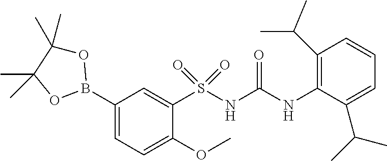 [SMILES: COc1ccc(B2OC(C)(C)C(C)(C)O2)cc1S(=O)(=O)NC(=O)Nc1c(C(C)C)cccc1C(C)C] | N-((2,6-diisopropylphenyl)carbamoyl)- 2-methoxy-5-(4,4,5,5-tetramethyl- 1,3,2-dioxaborolan-2- yl)benzenesulfonamide | 517 |
| 10 | 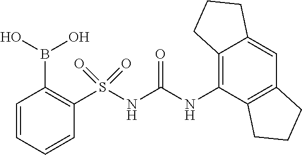 [SMILES: O=C(Nc1c2c(cc3c1CCC3)CCC2)NS(=O)(=O)c1ccccc1B(O)O] | (2-(N-((1,2,3,5,6,7-hexahydro-s- indacen-4- yl)carbamoyl)sulfamoyl)phenyl) boronic acid | 401 |
| 11 | 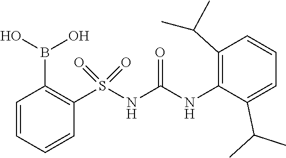 [SMILES: CC(C)c1cccc(C(C)C)c1NC(=O)NS(=O)(=O)c1ccccc1B(O)O] | (2-(N-((2,6-diisopropylphenyl) carbamoyl)sulfamoyl) phenyl)boronic acid | 535 |
| 12 | 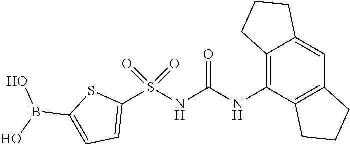 [SMILES: O=C(Nc1c2c(cc3c1CCC3)CCC2)NS(=O)(=O)c1ccc(B(O)O)s1] | (5-(N-((1,2,3,5,6,7-hexahydro-s- indacen-4- yl)carbamoyl)sulfamoyl)thiophen-2- yl)boronic acid | 407 |
| 13 | 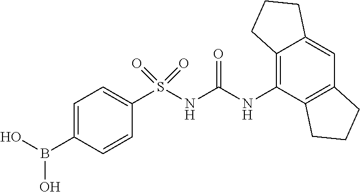 [SMILES: O=C(Nc1c2c(cc3c1CCC3)CCC2)NS(=O)(=O)c1ccc(B(O)O)cc1] | (4-(N-((1,2,3,5,6,7-hexahydro-s- indacen-4- yl)carbamoyl)sulfamoyl)phenyl) boronic acid | 401 |
| 14 | 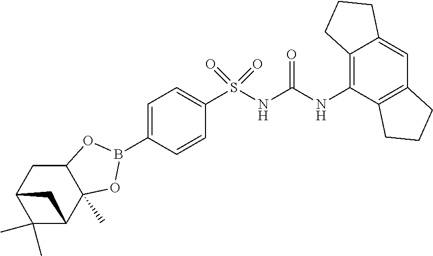 [SMILES: CC1(C)[C@@H]2CC3OB(c4ccc(S(=O)(=O)NC(=O)Nc5c6c(cc7c5CCC7)CCC6)cc4)O[C@@]3(C)[C@H]1C2] | N-((1,2,3,5,6,7-hexahydro-s-indacen-4- yl)carbamoyl)-4-((3aS,4S,6S)-3a,5,5- trimethylhexahydro-4,6- methanobenzo[d][1,3,2]dioxaborol-2- yl)benzenesulfonamide | 535 |
| 15 | 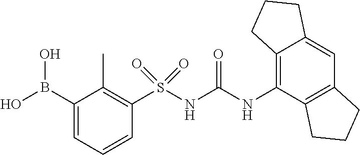 [SMILES: Cc1c(B(O)O)cccc1S(=O)(=O)NC(=O)Nc1c2c(cc3c1CCC3)CCC2] | (3-(N-((1,2,3,5,6,7-hexahydro-s- indacen-4-yl)carbamoyl)sulfamoyl)-2- methylphenyl)boronic acid | 415 |
| 16 | 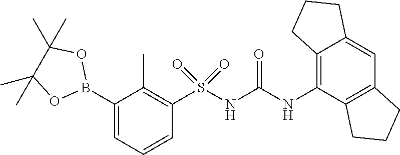 [SMILES: Cc1c(B2OC(C)(C)C(C)(C)O2)cccc1S(=O)(=O)NC(=O)Nc1c2c(cc3c1CCC3)CCC2] | N-((1,2,3,5,6,7-hexahydro-s-indacen-4- yl)carbamoyl)-2-methyl-3-(4,4,5,5- tetramethyl-1,3,2-dioxaborolan-2- yl)benzenesulfonamide | 497 |
| 17 | 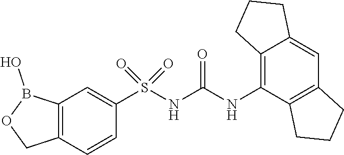 [SMILES: O=C(Nc1c2c(cc3c1CCC3)CCC2)NS(=O)(=O)c1ccc2c(c1)B(O)OC2] | N-((1,2,3,5,6,7-hexahydro-s-indacen-4- yl)carbamoyl)-1-hydroxy-1,3- dihydrobenzo[c][1,2]oxaborole-6- sulfonamide | 413 |
| 18 | 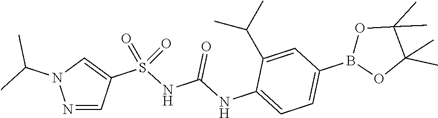 [SMILES: CC(C)c1cc(B2OC(C)(C)C(C)(C)O2)ccc1NC(=O)NS(=O)(=O)c1cnn(C(C)C)c1] | 1-isopropyl-N-((2-isopropyl-4-(4,4,5,5- tetramethyl-1,3,2-dioxaborolan-2- yl)phenyl)carbamoyl)-1H-pyrazole-4- sulfonamide | 477 |
| 19 | 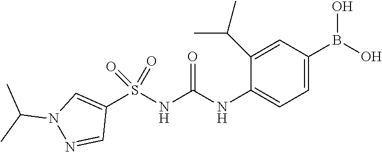 [SMILES: CC(C)c1cc(B(O)O)ccc1NC(=O)NS(=O)(=O)c1cnn(C(C)C)c1] | (3-isopropyl-4-(3-((1-isopropyl-1H- pyrazol-4- yl)sulfonyl)ureido)phenyl)boronic acid | 395 |
| 20 | 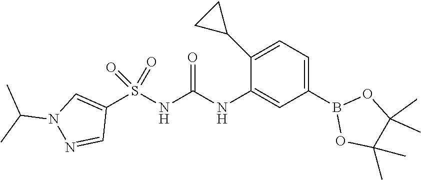 [SMILES: CC(C)n1cc(S(=O)(=O)NC(=O)Nc2cc(B3OC(C)(C)C(C)(C)O3)ccc2C2CC2)cn1] | N-((2-cyclopropyl-5-(4,4,5,5- tetramethyl-1,3,2-dioxaborolan-2- yl)phenyl)carbamoyl)-1-isopropyl-1H- pyrazole-4-sulfonamide | 475 |
| 21 | 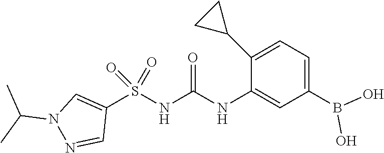 [SMILES: CC(C)n1cc(S(=O)(=O)NC(=O)Nc2cc(B(O)O)ccc2C2CC2)cn1] | (4-cyclopropyl-3-(3-((1-isopropyl-1H- pyrazol-4- yl)sulfonyl)ureido)phenyl)boronic acid | 393 |
| 22 | 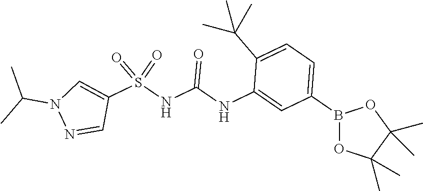 [SMILES: CC(C)n1cc(S(=O)(=O)NC(=O)Nc2cc(B3OC(C)(C)C(C)(C)O3)ccc2C(C)(C)C)cn1] | N-((2-(tert-butyl)-5-(4,4,5,5- tetramethyl-1,3,2-dioxaborolan-2- yl)phenyl)carbamoyl)-1-isopropyl-1H- pyrazole-4-sulfonamide | 491 |
| 23 | 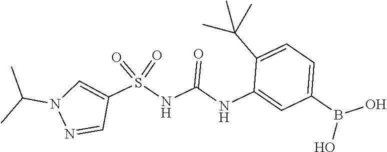 [SMILES: CC(C)n1cc(S(=O)(=O)NC(=O)Nc2cc(B(O)O)ccc2C(C)(C)C)cn1] | (4-(tert-butyl)-3-(3-((1-isopropyl-1H- pyrazol-4- yl)sulfonyl)ureido)phenyl)boronic acid | 409 |
| 24 | 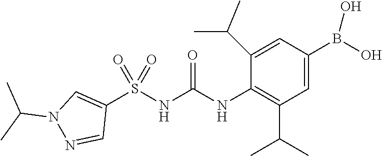 [SMILES: CC(C)c1cc(B(O)O)cc(C(C)C)c1NC(=O)NS(=O)(=O)c1cnn(C(C)C)c1] | (3,5-diisopropyl-4-(3-((1-isopropyl-1H- pyrazol-4- yl)sulfonyl)ureido)phenyl)boronic acid | 437 |
| 25 | 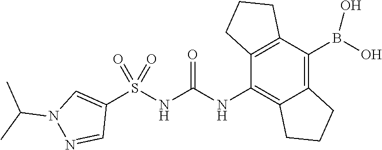 [SMILES: CC(C)n1cc(S(=O)(=O)NC(=O)Nc2c3c(c(B(O)O)c4c2CCC4)CCC3)cn1] | (8-(4-((1-isopropyl-1H-pyrazol-3- yl)sulfonyl)ureido)-1,2,3,5,6,7- hexahydro-s-indacen-4-yl)boronic acid | 433 |
| 26 | 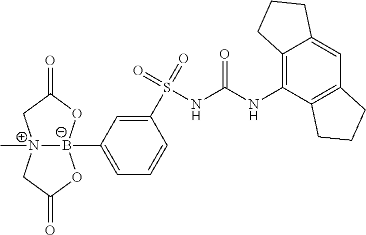 [SMILES: C[N+]12CC(=O)O[B-]1(c1cccc(S(=O)(=O)NC(=O)Nc3c4c(cc5c3CCC5)CCC4)c1)OC(=O)C2] | N-((1,2,3,5,6,7-hexahydro-s-indacen-4- yl)carbamoyl)-3-(4-methyl-2,6- dioxotetrahydro-2H-414,814- [1,3,2]oxazaborolo[2,3- b][1,3,2]oxazaborol-8- yl)benzenesulfonamide | 512.2 |
| 27 | 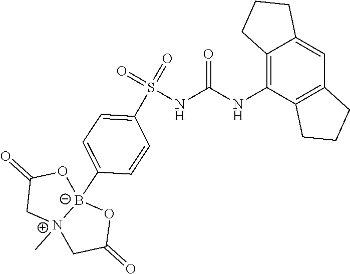 [SMILES: C[N+]12CC(=O)O[B-]1(c1ccc(S(=O)(=O)NC(=O)Nc3c4c(cc5c3CCC5)CCC4)cc1)OC(=O)C2] | N-((1,2,3,5,6,7-hexahydro-s-indacen-4- yl)carbamoyl)-4-(4-methyl-2,6- dioxotetrahydro-2H-414,814- [1,3,2]oxazaborolo[2,3- b][1,3,2]oxazaborol-8- yl)benzenesulfonamide | 512.2 |
| 28 | 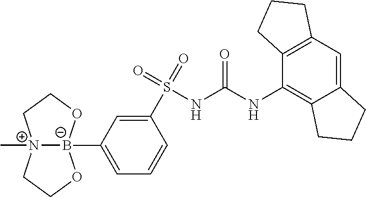 [SMILES: C[N+]12CCO[B-]1(c1cccc(S(=O)(=O)NC(=O)Nc3c4c(cc5c3CCC5)CCC4)c1)OCC2] | N-((1,2,3,5,6,7-hexahydro-s-indacen-4- yl)carbamoyl)-3-(4-methyltetrahydro- 2H-414,814-[1,3,2]oxazaborolo[2,3- b][1,3,2]oxazaborol-8- yl)benzenesulfonamide | 401.1 |
| 29 | 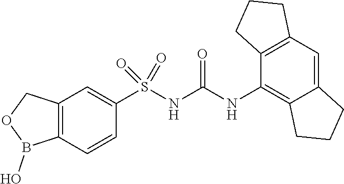 [SMILES: O=C(Nc1c2c(cc3c1CCC3)CCC2)NS(=O)(=O)c1ccc2c(c1)COB2O] | N-((1,2,3,5,6,7-hexahydro-s-indacen-4- yl)carbamoyl)-1-hydroxy-1,3- dihydrobenzo[c][1,2]oxaborole-5- sulfonamide | 412.8 |
| 30 | 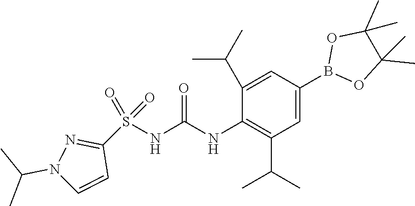 [SMILES: CC(C)c1cc(B2OC(C)(C)C(C)(C)O2)cc(C(C)C)c1NC(=O)NS(=O)(=O)c1ccn(C(C)C)n1] | N-((2,6-diisopropyl-4-(4,4,5,5- tetramethyl-1,3,2-dioxaborolan-2- yl)phenyl)carbamoyl)-1-isopropyl-1H- pyrazole-3-sulfonamide | 417.3/504.3 |
| 31 | 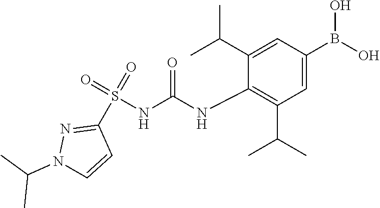 [SMILES: CC(C)c1cc(B(O)O)cc(C(C)C)c1NC(=O)NS(=O)(=O)c1ccn(C(C)C)n1] | (3,5-diisopropyl-4-(3-((1-isopropyl-1H- pyrazol-3- yl)sulfonyl)ureido)phenyl)boronic acid | 437.2 |
| 32 | 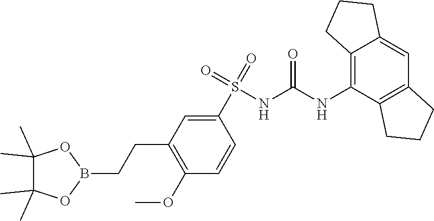 [SMILES: COc1ccc(S(=O)(=O)NC(=O)Nc2c3c(cc4c2CCC4)CCC3)cc1CCB1OC(C)(C)C(C)(C)O1] | N-((1,2,3,5,6,7-hexahydro-s-indacen-4- yl)carbamoyl)-4-methoxy-3-(2- (4,4,5,5-tetramethyl-1,3,2- dioxaborolan-2- yl)ethyl)benzenesulfonamide | 541.2 |
| 33 | 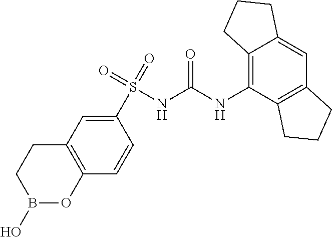 [SMILES: O=C(Nc1c2c(cc3c1CCC3)CCC2)NS(=O)(=O)c1ccc2c(c1)CCB(O)O2] | N-((1,2,3,5,6,7-hexahydro-s-indacen-4- yl)carbamoyl)-2-hydroxy-3,4-dihydro- 2H-benzo[e][1,2]oxaborinine-6- sulfonamide | 427.0 |
| 34 | 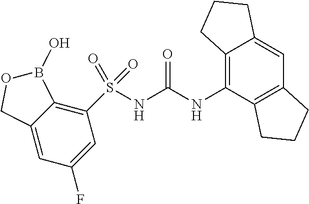 [SMILES: O=C(Nc1c2c(cc3c1CCC3)CCC2)NS(=O)(=O)c1cc(F)cc2c1B(O)OC2] | 5-fluoro-N-((1,2,3,5,6,7-hexahydro-s- indacen-4-yl)carbamoyl)-1-hydroxy- 1,3-dihydrobenzo[c][1,2]oxaborole-7- sulfonamide | 431.1 |
| 35 | 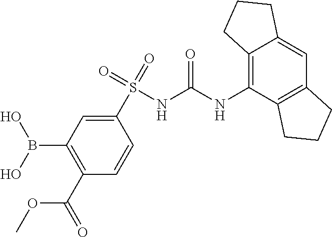 [SMILES: COC(=O)c1ccc(S(=O)(=O)NC(=O)Nc2c3c(cc4c2CCC4)CCC3)cc1B(O)O] | (5-(N-((1,2,3,5,6,7-hexahydro-s- indacen-4-yl)carbamoyl)sulfamoyl)-2- (methoxycarbonyl)phenyl)boronic acid | 459.2 |
| 36 | 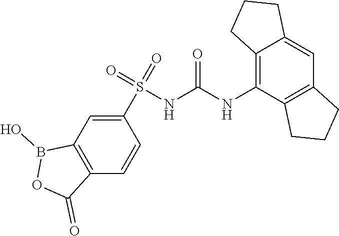 [SMILES: O=C(Nc1c2c(cc3c1CCC3)CCC2)NS(=O)(=O)c1ccc2c(c1)B(O)OC2=O] | N-((1,2,3,5,6,7-hexahydro-s-indacen-4- yl)carbamoyl)-1-hydroxy-3-oxo-1,3- dihydrobenzo[c][1,2]oxaborole-6- sulfonamide | 427.2 |
| 37 | 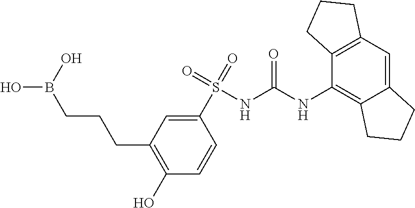 [SMILES: O=C(Nc1c2c(cc3c1CCC3)CCC2)NS(=O)(=O)c1ccc(O)c(CCCB(O)O)c1] | (3-(5-(N-((1,2,3,5,6,7-hexahydro-s- indacen-4-yl)carbamoyl)sulfamoyl)-2- hydroxyphenyl)propyl)boronic acid | 459.1 |
| 38 | 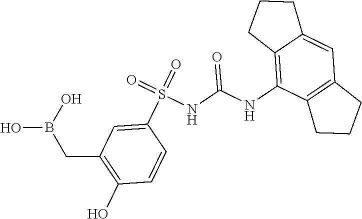 [SMILES: O=C(Nc1c2c(cc3c1CCC3)CCC2)NS(=O)(=O)c1ccc(O)c(CB(O)O)c1] | (5-(N-((1,2,3,5,6,7-hexahydro-s- indacen-4-yl)carbamoyl)sulfamoyl)-2- hydroxybenzyl)boronic acid | 431.1 |
| 39 | 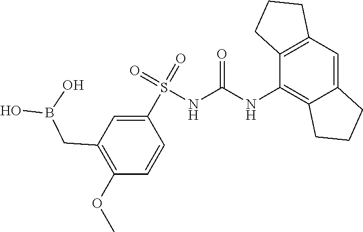 [SMILES: COc1ccc(S(=O)(=O)NC(=O)Nc2c3c(cc4c2CCC4)CCC3)cc1CB(O)O] | (5-(N-((1,2,3,5,6,7-hexahydro-s- indacen-4-yl)carbamoyl)sulfamoyl)-2- methoxybenzyl)boronic acid | 445.2 |
| 40 | 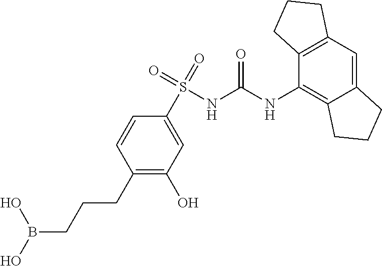 [SMILES: O=C(Nc1c2c(cc3c1CCC3)CCC2)NS(=O)(=O)c1ccc(CCCB(O)O)c(O)c1] | (3-(4-(N-((1,2,3,5,6,7-hexahydro-s- indacen-4-yl)carbamoyl)sulfamoyl)-2- hydroxyphenyl)propyl)boronic acid | 459.2 |
| 41 | 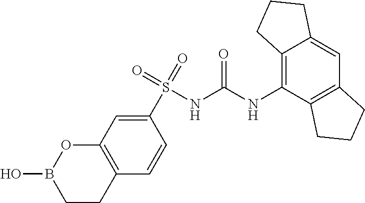 [SMILES: O=C(Nc1c2c(cc3c1CCC3)CCC2)NS(=O)(=O)c1ccc2c(c1)OB(O)CC2] | N-((1,2,3,5,6,7-hexahydro-s-indacen-4- yl)carbamoyl)-2-hydroxy-3,4-dihydro- 2H-benzo[e][1,2]oxaborinine-7- sulfonamide | 427.2 |
| 42 | 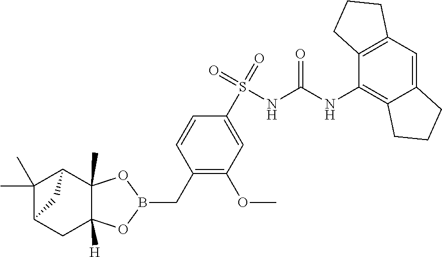 [SMILES: COc1cc(S(=O)(=O)NC(=O)Nc2c3c(cc4c2CCC4)CCC3)ccc1CB1O[C@@H]2C[C@@H]3C[C@@H](C3(C)C)[C@]2(C)O1] | N-((1,2,3,5,6,7-hexahydro-s-indacen-4- yl)carbamoyl)-3-methoxy-4- (((3aS,4S,6S,7aR)-3a,5,5- trimethylhexahydro-4,6- methanobenzo[d][1,3,2]dioxaborol-2- yl)methyl)benzenesulfonamide | 579.3 |
| 43 | 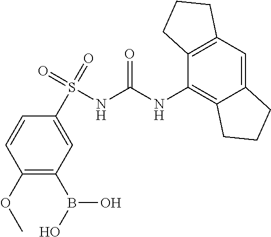 [SMILES: COc1ccc(S(=O)(=O)NC(=O)Nc2c3c(cc4c2CCC4)CCC3)cc1B(O)O] | (5-(N-((1,2,3,5,6,7-hexahydro-s- indacen-4-yl)carbamoyl)sulfamoyl)-2- methoxyphenyl)boronic acid | 431.1 |
| 44 | 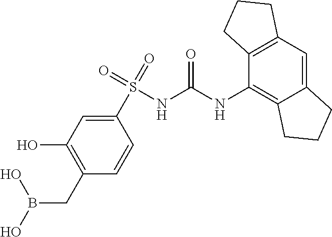 [SMILES: O=C(Nc1c2c(cc3c1CCC3)CCC2)NS(=O)(=O)c1ccc(CB(O)O)c(O)c1] | (4-(N-((1,2,3,5,6,7-hexahydro-s- indacen-4-yl)carbamoyl)sulfamoyl)-2- hydroxybenzyl)boronic acid | 431.2 |
| 45 | 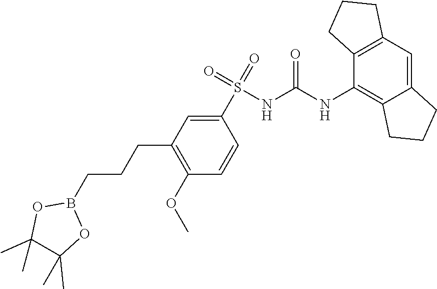 [SMILES: COc1ccc(S(=O)(=O)NC(=O)Nc2c3c(cc4c2CCC4)CCC3)cc1CCCB1OC(C)(C)C(C)(C)O1] | N-((1,2,3,5,6,7-hexahydro-s-indacen-4- yl)carbamoyl)-4-methoxy-3-(3- (4,4,5,5-tetramethyl-1,3,2- dioxaborolan-2- yl)propyl)benzenesulfonamide | 555.3 |
| 46 | 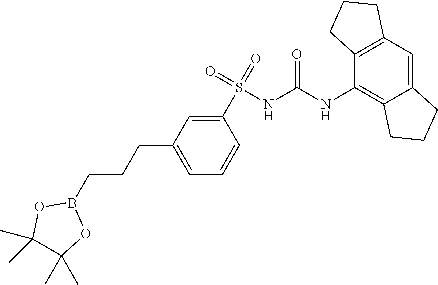 [SMILES: CC1(C)OB(CCCc2cccc(S(=O)(=O)NC(=O)Nc3c4c(cc5c3CCC5)CCC4)c2)OC1(C)C] | N-((1,2,3,5,6,7-hexahydro-s-indacen-4- yl)carbamoyl)-3-(3-(4,4,5,5- tetramethyl-1,3,2-dioxaborolan-2- yl)propyl)benzenesulfonamide | 525.1 |
| 47 | 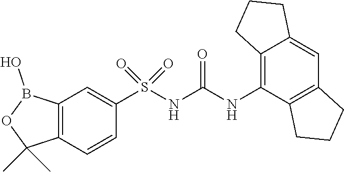 [SMILES: CC1(C)OB(O)c2cc(S(=O)(=O)NC(=O)Nc3c4c(cc5c3CCC5)CCC4)ccc21] | N-((1,2,3,5,6,7-hexahydro-s-indacen-4- yl)carbamoyl)-1-hydroxy-3,3-dimethyl- 1,3-dihydrobenzo[c][1,2]oxaborole-6- sulfonamide | 441.2 |
| 48 | 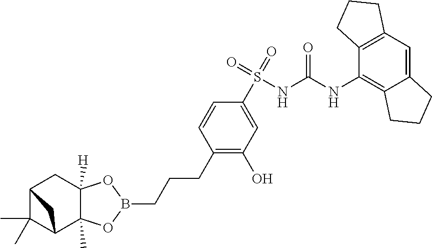 [SMILES: CC1(C)[C@@H]2C[C@H]3OB(CCCc4ccc(S(=O)(=O)NC(=O)Nc5c6c(cc7c5CCC7)CCC6)cc4O)O[C@@]3(C)[C@H]1C2] | N-((1,2,3,5,6,7-hexahydro-s-indacen-4- yl)carbamoyl)-3-hydroxy-4-(3- ((3aS,4S,6S,7aR)-3a,5,5- trimethylhexahydro-4,6- methanobenzo[d][1,3,2]dioxaborol-2- yl)propyl)benzenesulfonamide | 593.3 |
| 49 | 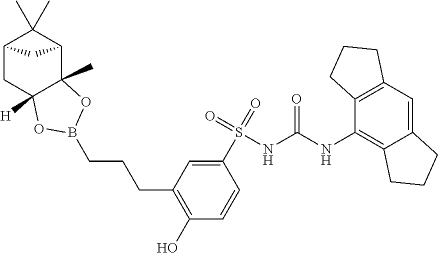 [SMILES: CC1(C)[C@@H]2C[C@H]3OB(CCCc4cc(S(=O)(=O)NC(=O)Nc5c6c(cc7c5CCC7)CCC6)ccc4O)O[C@@]3(C)[C@H]1C2] | N-((1,2,3,5,6,7-hexahydro-s-indacen-4- yl)carbamoyl)-4-hydroxy-3-(3- ((3aS,4S,6S,7aR)-3a,5,5- trimethylhexahydro-4,6- methanobenzo[d][1,3,2]dioxaborol-2- yl)propyl)benzenesulfonamide | 593.3 |
| 50 | 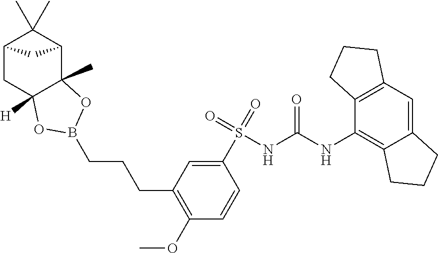 [SMILES: COc1ccc(S(=O)(=O)NC(=O)Nc2c3c(cc4c2CCC4)CCC3)cc1CCCB1O[C@@H]2C[C@@H]3C[C@@H](C3(C)C)[C@]2(C)O1] | N-((1,2,3,5,6,7-hexahydro-s-indacen-4- yl)carbamoyl)-4-methoxy-3-(3- ((3aS,4S,6S,7aR)-3a,5,5- trimethylhexahydro-4,6- methanobenzo[d][1,3,2]dioxaborol-2- yl)propyl)benzenesulfonamide | 607.3 |
| 51 | 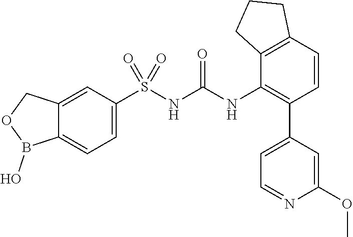 [SMILES: COc1cc(-c2ccc3c(c2NC(=O)NS(=O)(=O)c2ccc4c(c2)COB4O)CCC3)ccn1] | 1-hydroxy-N-((5-(2-methoxypyridin-4- yl)-2,3-dihydro-1H-inden-4- yl)carbamoyl)-1,3- dihydrobenzo[c][1,2]oxaborole-5- sulfonamide | 479.8 |
| 52 | 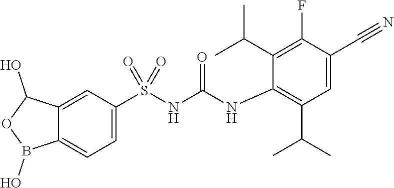 [SMILES: CC(C)c1cc(C#N)c(F)c(C(C)C)c1NC(=O)NS(=O)(=O)c1ccc2c(c1)C(O)OB2O] | N-((4-cyano-3-fluoro-2,6- diisopropylphenyl)carbamoyl)-1,3- dihydroxy-1,3- dihydrobenzo[c][1,2]oxaborole-5- sulfonamide | 475.9 |
| 53 | 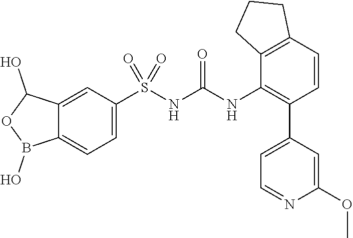 [SMILES: COc1cc(-c2ccc3c(c2NC(=O)NS(=O)(=O)c2ccc4c(c2)C(O)OB4O)CCC3)ccn1] | 1,3-dihydroxy-N-((5-(2- methoxypyridin-4-yl)-2,3-dihydro-1H- inden-4-yl)carbamoyl)-1,3- dihydrobenzo[c][1,2]oxaborole-5- sulfonamide | 495.9 |
| 54 | 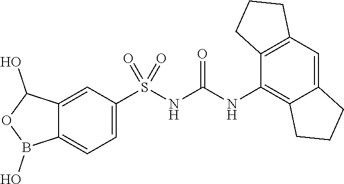 [SMILES: O=C(Nc1c2c(cc3c1CCC3)CCC2)NS(=O)(=O)c1ccc2c(c1)C(O)OB2O] | N-((1,2,3,5,6,7-hexahydro-s-indacen-4- yl)carbamoyl)-1,3-dihydroxy-1,3- dihydrobenzo[c][1,2]oxaborole-5- sulfonamide | 428.9 |
| 55 | 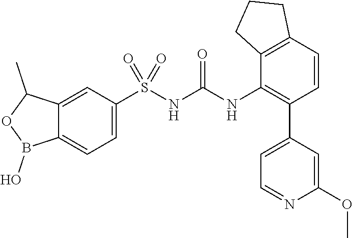 [SMILES: COc1cc(-c2ccc3c(c2NC(=O)NS(=O)(=O)c2ccc4c(c2)C(C)OB4O)CCC3)ccn1] | 1-hydroxy-N-((5-(2-methoxypyridin-4- yl)-2,3-dihydro-1H-inden-4- yl)carbamoyl)-3-methyl-1,3- dihydrobenzo[c][1,2]oxaborole-5- sulfonamide | 493.8 |
| 56 | 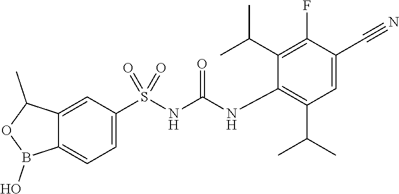 [SMILES: CC(C)c1cc(C#N)c(F)c(C(C)C)c1NC(=O)NS(=O)(=O)c1ccc2c(c1)C(C)OB2O] | N-((4-cyano-3-fluoro-2,6- diisopropylphenyl)carbamoyl)-1- hydroxy-3-methyl-1,3- dihydrobenzo[c][1,2]oxaborole-5- sulfonamide | 473.9 |
| 57 | 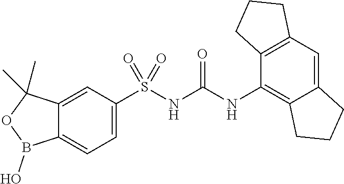 [SMILES: CC1(C)OB(O)c2ccc(S(=O)(=O)NC(=O)Nc3c4c(cc5c3CCC5)CCC4)cc21] | N-((1,2,3,5,6,7-hexahydro-s-indacen-4- yl)carbamoyl)-1-hydroxy-3,3-dimethyl- 1,3-dihydrobenzo[c][1,2]oxaborole-5- sulfonamide | 440.7 |
| 58 | 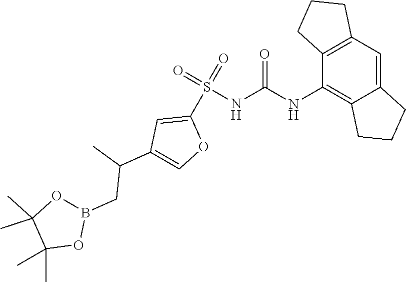 [SMILES: CC(CB1OC(C)(C)C(C)(C)O1)c1coc(S(=O)(=O)NC(=O)Nc2c3c(cc4c2CCC4)CCC3)c1] | N-((1,2,3,5,6,7-hexahydro-s-indacen-4- yl)carbamoyl)-4-(1-(4,4,5,5- tetramethyl-1,3,2-dioxaborolan-2- yl)propan-2-yl)furan-2-sulfonamide | 515.3 |
| 59 | 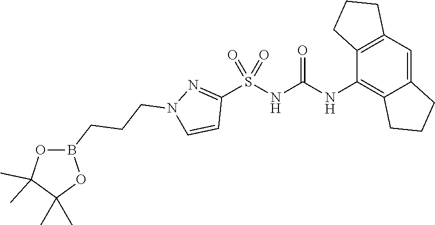 [SMILES: CC1(C)OB(CCCn2ccc(S(=O)(=O)NC(=O)Nc3c4c(cc5c3CCC5)CCC4)n2)OC1(C)C] | N-((1,2,3,5,6,7-hexahydro-s-indacen-4- yl)carbamoyl)-1-(3-(4,4,5,5- tetramethyl-1,3,2-dioxaborolan-2- yl)propyl)-1H-pyrazole-3-sulfonamide | 515.2 |
| 60 | 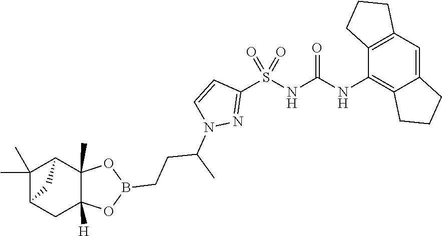 [SMILES: CC(CCB1O[C@@H]2C[C@@H]3C[C@@H](C3(C)C)[C@]2(C)O1)n1ccc(S(=O)(=O)NC(=O)Nc2c3c(cc4c2CCC4)CCC3)n1] | N-((1,2,3,5,6,7-hexahydro-s-indacen-4- yl)carbamoyl)-1-(4-((3aS,4S,6S,7aR)- 3a,5,5-trimethylhexahydro-4,6- methanobenzo[d][1,3,2]dioxaborol-2- yl)butan-2-yl)-1H-pyrazole-3- sulfonamide | 581.2 |
| 61 | 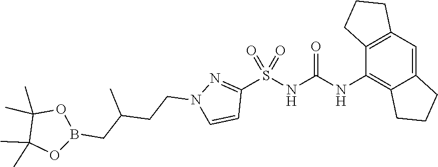 [SMILES: CC(CCn1ccc(S(=O)(=O)NC(=O)Nc2c3c(cc4c2CCC4)CCC3)n1)CB1OC(C)(C)C(C)(C)O1] | N-((1,2,3,5,6,7-hexahydro-s-indacen-4- yl)carbamoyl)-1-(3-methyl-4-(4,4,5,5- tetramethyl-1,3,2-dioxaborolan-2- yl)butyl)-1H-pyrazole-3-sulfonamide | 543.2 |
| 62 | 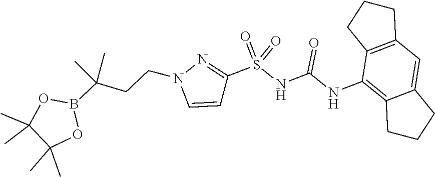 [SMILES: CC(C)(CCn1ccc(S(=O)(=O)NC(=O)Nc2c3c(cc4c2CCC4)CCC3)n1)B1OC(C)(C)C(C)(C)O1] | N-((1,2,3,5,6,7-hexahydro-s-indacen-4- yl)carbamoyl)-1-(3-methyl-3-(4,4,5,5- tetramethyl-1,3,2-dioxaborolan-2- yl)butyl)-1H-pyrazole-3-sulfonamide | 543.2 |
| 63 | 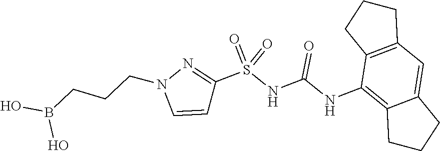 [SMILES: O=C(Nc1c2c(cc3c1CCC3)CCC2)NS(=O)(=O)c1ccn(CCCB(O)O)n1] | (3-(3-(N-((1,2,3,5,6,7-hexahydro-s- indacen-4-yl)carbamoyl)sulfamoyl)- 1H-pyrazol-1-yl)propyl)boronic acid | 415.1 |
| 64 | 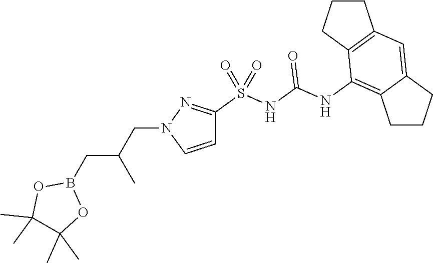 [SMILES: CC(CB1OC(C)(C)C(C)(C)O1)Cn1ccc(S(=O)(=O)NC(=O)Nc2c3c(cc4c2CCC4)CCC3)n1] | N-((1,2,3,5,6,7-hexahydro-s-indacen-4- yl)carbamoyl)-1-(2-methyl-3-(4,4,5,5- tetramethyl-1,3,2-dioxaborolan-2- yl)propyl)-1H-pyrazole-3-sulfonamide | 529.3 |
| 65 | 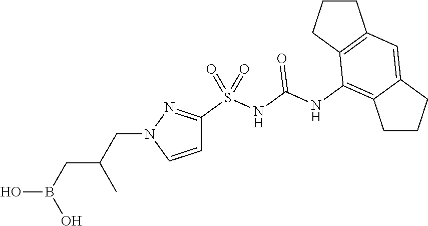 [SMILES: CC(CB(O)O)Cn1ccc(S(=O)(=O)NC(=O)Nc2c3c(cc4c2CCC4)CCC3)n1] | (3-(3-(N-((1,2,3,5,6,7-hexahydro-s- indacen-4-yl)carbamoyl)sulfamoyl)- 1H-pyrazol-1-yl)-2- methylpropyl)boronic acid | 469.2 (M + Na)⁺ |
| 66 | 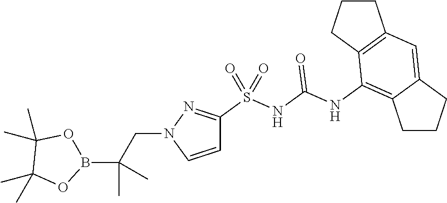 [SMILES: CC(C)(Cn1ccc(S(=O)(=O)NC(=O)Nc2c3c(cc4c2CCC4)CCC3)n1)B1OC(C)(C)C(C)(C)O1] | N-((1,2,3,5,6,7-hexahydro-s-indacen-4- yl)carbamoyl)-1-(2-methyl-2-(4,4,5,5- tetramethyl-1,3,2-dioxaborolan-2- yl)propyl)-1H-pyrazole-3-sulfonamide | 529.3 |
| 67 | 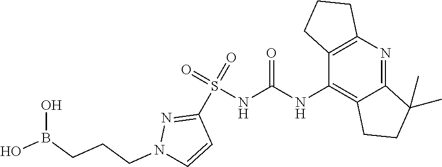 [SMILES: CC1(C)CCc2c1nc1c(c2NC(=O)NS(=O)(=O)c2ccn(CCCB(O)O)n2)CCC1] | (3-(3-(N-((3,3-dimethyl-1,2,3,5,6,7- hexahydrodicyclopenta[b,e]pyridin-8- yl)carbamoyl)sulfamoyl)-1H-pyrazol- 1-yl)propyl)boronic acid | 462.2 |
| 68 | 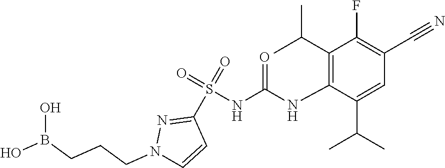 [SMILES: CC(C)c1cc(C#N)c(F)c(C(C)C)c1NC(=O)NS(=O)(=O)c1ccn(CCCB(O)O)n1] | (3-(3-(N-((4-cyano-3-fluoro-2,6- diisopropylphenyl)carbamoyl)sulfamoyl)- 1H-pyrazol-1-yl)propyl)boronic acid | 462.1 |
| 69 | 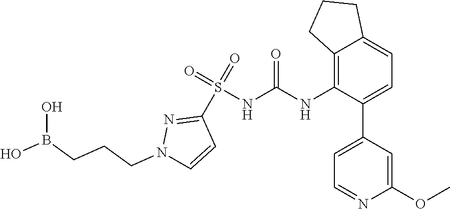 [SMILES: COc1cc(-c2ccc3c(c2NC(=O)NS(=O)(=O)c2ccn(CCCB(O)O)n2)CCC3)ccn1] | (3-(3-(N-((5-(2-methoxypyridin-4-yl)- 2,3-dihydro-1H-inden-4- yl)carbamoyl)sulfamoyl)-1H-pyrazol- 1-yl)propyl)boronic acid | 500.1 |
| 70 | 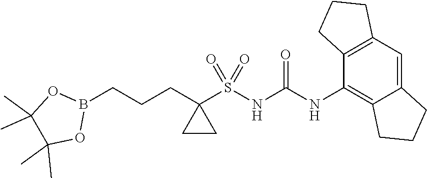 [SMILES: CC1(C)OB(CCCC2(S(=O)(=O)NC(=O)Nc3c4c(cc5c3CCC5)CCC4)CC2)OC1(C)C] | N-((1,2,3,5,6,7-hexahydro-s-indacen-4- yl)carbamoyl)-1-(3-(4,4,5,5- tetramethyl-1,3,2-dioxaborolan-2- yl)propyl)cyclopropane-1-sulfonamide | 489.3 |
| 71 | 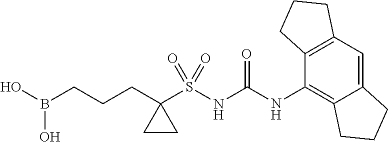 [SMILES: O=C(Nc1c2c(cc3c1CCC3)CCC2)NS(=O)(=O)C1(CCCB(O)O)CC1] | (3-(1-(N-((1,2,3,5,6,7-hexahydro-s- indacen-4- yl)carbamoyl)sulfamoyl)cyclopropyl) propyl)boronic acid | 429.2 (M + Na)⁺ |
| 72 | 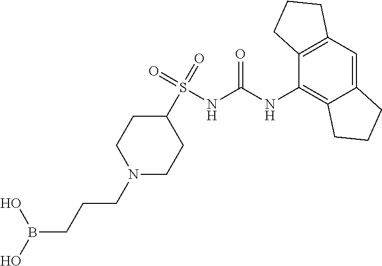 [SMILES: O=C(Nc1c2c(cc3c1CCC3)CCC2)NS(=O)(=O)C1CCN(CCCB(O)O)CC1] | (3-(4-(N-((1,2,3,5,6,7-hexahydro-s- indacen-4- yl)carbamoyl)sulfamoyl)piperidin-1- yl)propyl)boronic acid | 450.3 |
| 73 | 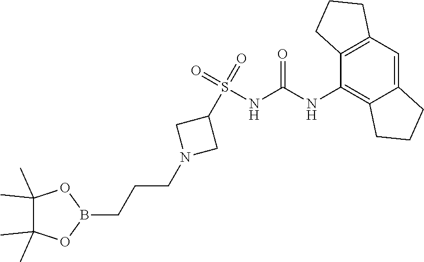 [SMILES: CC1(C)OB(CCCN2CC(S(=O)(=O)NC(=O)Nc3c4c(cc5c3CCC5)CCC4)C2)OC1(C)C] | N-((1,2,3,5,6,7-hexahydro-s-indacen-4- yl)carbamoyl)-1-(3-(4,4,5,5- tetramethyl-1,3,2-dioxaborolan-2- yl)propyl)azetidine-3-sulfonamide | 504.1 |

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein include isotopes of hydrogen, carbon, nitrogen, oxygen, phosphorous, sulfur, fluorine and chloride, such as ²H, ³H, ¹³C, ¹⁴C, ¹⁵N, ¹⁸O, ¹⁷O, ³¹P, ³²p, ³⁵S, ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or a solvate, or stereoisomer thereof, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein, solvate, or stereoisomer thereof and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared by recrystallization from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Method of Treatment

Disclosed herein are methods comprising administering an NLRP3 inflammasome inhibitor to a subject exhibiting increased levels of NLRP3 inflammasome activity or determined to be at risk for developing increased levels of NLRP3 inflammasome activity. Also disclosed herein are methods comprising administering an NLRP3 inflammasome inhibitor to a subject that has been diagnosed with an NLRP3 inflammasome-related disease or disorder, or who has symptoms or signs of an NLRP3 inflammasome-related disease or disorder.

In some embodiments inhibiting NLRP3 inflammasome activity is accomplished using any method known to the skilled artisan. Examples of methods to inhibit NLRP3 inflammasome activity include, but are not limited to, directly blocking the assembly of the NLRP3 inflammasome complex by inhibiting the oligomerization of inflammasome adaptor protein ASC (also called PY CARD (PYD and CARD domain containing)), decreasing expression of an endogenous NLRP3 inflammasome gene, decreasing expression of NLRP3 inflammasome mRNA, and inhibiting activity of NLRP3 inflammasome protein, Decreasing expression of an endogenous NLRP3 inflammasome gene includes providing a specific inhibitor of NLRP3 inflammasome gene expression. Decreasing expression of NLRP3 inflammasome mRNA or NLRP3 inflammasome protein includes decreasing the half-life or stability of NLRP3 inflammasome mRNA or decreasing expression of NLRP3 inflammasome mRNA. In some embodiments, the NLRP3 inflammasome inhibitor is a compound that decreases expression of an NLRP3 inflammasome gene, that decreases NLRP3 inflammasome mRNA half-life, stability and/or expression, or that inhibits NLRP3 inflammasome protein function. In some embodiments, the inhibitory effect of a therapeutic agent on NLRP3 inflammasome expression, function, or activity is indirect.

Provided herein are methods of inhibiting the NLRP3 inflammasome, which is useful for treating, preventing, or ameliorating a disease associated with the NLRP3 inflammasome in a subject in need thereof, by administration of a compound disclosed herein, or a pharmaceutically acceptable salt, solvate, or stereoisomer thereof.

In some embodiments, the disease is responsive to modulation of one or more of IL-6, IL-1 beta, IL-17, IL-18, IL-1 alpha, IL-37, IL-22, IL-33 and Th17 cells.

In some embodiments, the modulation is inhibition of one or more of IL-6, IL-1 beta, IL-17, IL-18, IL-1 alpha, IL-37, IL-22, IL-33.

In some embodiments, the modulation of Th17 cells, is by inhibition of production and/or secretion of IL-17.

In some embodiments, the disease is an immune system disease, an inflammatory disease, an autoimmune disease, a skin disease, a cardiovascular disease, cancer, a renal system disease, a gastro-intestinal tract disease, a respiratory system disease, a disease of the endocrine system, a viral disease, or a central nervous system (CNS) disease.

In some embodiments, the disease is an immune system disease. In some embodiments, the disease is an inflammatory disease. In some embodiments, the disease is an autoimmune disease. In some embodiments, the disease is of the skin. In some embodiments, the disease is a cardiovascular disease. In some embodiments, the disease is a viral disease. In some embodiments, the disease is a renal system disease. In some embodiments, the disease is a gastro-intestinal tract disease. In some embodiments, the disease is a respiratory system disease. In some embodiments, the disease is a disease of the endocrine system. In some embodiments, the disease is a viral disease. In some embodiments, the disease is a central nervous system (CNS) disease.

In some embodiments, the disease is a cancer, tumor, or other malignancy. As used herein, cancers tumors and malignancies, refer to diseases disorders or conditions, or to cells or tissues associated with the diseases, disorders or conditions, characterized by aberrant or abnormal cell proliferation, differentiation and/or migration often accompanied by an aberrant or abnormal molecular phenotype that includes one or more genetic mutations or other genetic changes associated with oncogenesis, expression of tumor markers, loss of tumor suppressor expression or activity and/or aberrant or abnormal cell surface marker expression. In general embodiments, cancers, tumors and malignancies may include sarcomas, lymphomas, leukemias, solid tumors, blastomas, gliomas, carcinomas, melanomas and metastatic cancers, although without limitation thereto.

In some embodiments, the disease is caused by, or is associated with, a pathogen. In some embodiments, the pathogen is a virus, a bacterium, a protist, a worm, or a fungus.

Non-limiting examples of viruses include influenza virus, cytomegalovirus, Epstein Barr Virus, human immunodeficiency virus (HIV), alphaviruses such as Chikungunya and Ross River virus, flaviviruses such as Dengue virus, Zika virus and papillomavirus, and coronaviruses such as 229E (alpha coronavirus), NL63 (alpha coronavirus), OC43 (beta coronavirus), HKU1 (beta coronavirus), MERS-CoV (the beta coronavirus that causes Middle East Respiratory Syndrome, or MERS), SARS-CoV (the beta coronavirus that causes severe acute respiratory syndrome, or SARS), and SARS-CoV-2 (the novel coronavirus that causes coronavirus disease 2019, or COVID-19).

Non-limiting examples of pathogenic bacteria include Staphylococcus aureus, Helicobacter pylori, Bacillus anthracis, Bordatella pertussis, Corynebacterium diptheriae, Clostridium tetani, Clostridium botulinum, Streptococcus pneumoniae, Streptococcus pyogenes, Listeria monocytogenes, Hemophilus influenzae, Pasteurella multicida, Shigella dysenteriae, Mycobacterium tuberculosis, Mycobacterium leprae, Mycoplasma pneumoniae, Mycoplasma hominis, Neisseria meningitidis, Neisseria gonorrhoeae, Rickettsia rickettsii, Legionella pneumophila, Klebsiella pneumoniae, Pseudomonas aeruginosa, Propionibacterium acnes, Treponema pallidum, Chlamydia trachomatis, Vibrio cholerae, Salmonella typhimurium, Salmonella typhi, Borrelia burgdorferi and Yersinia pestis.

Non-limiting examples of protists include Plasmodium, Babesia, Giardia, Entamoeba, Leishmania and Trypanosomes.

Non-limiting examples of worms include helminths inclusive of schistisimes, roundworms, tapeworms and flukes.

Non-limiting examples of fungi include Candida and Aspergillus species.

In some embodiments, the disease is constitutive inflammation including, viral- or pathogen-associated hyperinflammation, cytokine release syndrome, acute respiratory distress syndrome, acute lung; injury, septic shock, macrophage activating syndrome, hemophagocytic lymphohistiocytosis, and coronavius disease; cryopyrin-associated periodic syndromes (CAPS), Muckle-Wells syndrome (MWS), familial cold autoinflammatory syndrome (FCAS) and neonatal-onset multisystem inflammatory disease (NOMID), autoinflammatory diseases, familial Mediterranean fever (FMF) TNF receptor associated periodic syndrome (TRAPS), mevalonate kinase deficiency (MKD), hyperimmunoglobulinemia D and periodic fever syndrome (HIDS), deficiency of interleukin I receptor (DIRA) antagonist, Majeed syndrome, pyogenic arthritis, pyoderna gangrenosum and acne syndrome (PAPA), haploinsufficiency of A20 (HA20), pediatric granulomatous arthritis (PGA), PLCG2-associated antibody deficiency and immune dysregulation (PLAID), PLCG2-associated autoinflammation, antibody deficiency and immune dysregulation (APLATD) and sideroblastic anemia with B-cell immunodeficiency, periodic fevers, and developmental delay (SIFD); autoimmune diseases including multiple sclerosis (MS), type-1 diabetes, psoriasis, rheumatoid arthritis, Behcet's disease, Sjogren's syndrome and Schnitzler syndrome; macrophage activation syndrome; Blau syndrome; respiratory diseases including chronic obstructive pulmonary disorder (COPD), asthma such as allergic asthma and steroid-resistant asthma, asbestosis, silicosis and cystic fibrosis; dermatitis including contact dermatitis; central nervous system diseases including Parkinson's disease, Alzheimer's disease, motor neuron disease, Huntington's disease, cerebral malaria and brain injury from pneumococcal meningitis; metabolic diseases including Type 2 diabetes, atherosclerosis, obesity, gout, pseudo-gout; ocular diseases including those of the ocular epithelium, age-related macular degeneration (AMD), uveitis, corneal infection and dry eye; kidney disease including chronic kidney disease, oxalate nephropathy, nephrocalcinosis and diabetic nephropathy; liver disease including non-alcoholic steatohepatitis (NASH) and alcoholic liver disease; inflammatory reactions in skin including contact hypersensitivity and sunburn; inflammatory reactions in the joints including; osteoarthritis, systemic juvenile idiopathic arthritis, adult-onset Still's disease, relapsing polychondritis; viral infections including alpha virus (Chikungunya, Ross River) and flavivirus (Dengue, Zika), flu, HIV; hidradenitis suppurativa (HS) and other cyst-causing skin diseases: cancers including lung cancer metastasis, pancreatic cancers, gastric cancers, myelodysplastic syndrome, leukemia: polymyositis; stroke including ischemic stroke; myocardial infarction including recurrent myocardial infarction; congestive heart failure; embolism; cardiovascular disease; Graft versus Host Disease; hypertension; colitis; helminth infection; bacterial infection; abdominal aortic aneurism; wound healing; depression, psychological stress; ischemia reperfusion injury, or diseases where an individual has been determined to carry a germline or somatic non-silent mutation in NLRP3.

In some embodiments, the disease being treated is NASH. NLRP3 inflammasome activation is central to inflammatory recruitment in NASH, and inhibition of NLRP3 may both prevent and reverse liver fibrosis. In some embodiments, the compounds disclosed herein cause histological reductions in liver inflammation, decreased recruitment of macrophages and neutrophils, and suppression of NF-κB activation interrupting the function of NLRP3 inflammasomes in liver tissue.

In some embodiments, inhibition of the NLRP3 reduces hepatic expression of pro-IL-1 beta and normalized hepatic and circulating IL-1 beta, IL-6 and MCP-1 levels thereby assisting in treatment of the disease.

In some embodiments, the disease is severe steroid resistant (SSR) asthma. Respiratory infections induce an NLRP3 inflammasome/caspase-1/IL-1 beta signaling axis in the lungs that promotes SSR asthma. The NLRP3 inflammasome recruits, and activates, pro-caspase-1 to induce IL-1 beta responses. NLRP3 inflammasome-induced IL-1 beta responses are therefore important in the control of infections, however, excessive activation results in aberrant inflammation and has been associated with the pathogenesis of SSR asthma and COPD. The administration of compounds described herein that target specific disease processes, are more therapeutically attractive than non-specifically inhibiting inflammatory responses with steroids or IL-1 beta. In some embodiments, targeting the NLRP3 inflammasome/caspase-1/IL-1 beta signaling axis with the compounds described herein is useful in the treatment of SSR asthma and other steroid-resistant inflammatory conditions. In some embodiments, the disease is Parkinson's disease. Parkinson's is the most common neurodegenerative movement disorder and is characterized by a selective loss of dopaminergic neurons, accompanied by the accumulation of mis-folded a-synuclein (Syn) into Lewy bodies that are pathological hallmarks of the disease. Chronic microglial neuroinflammation is evident early in the disease, and has been proposed to drive pathology.

### Dosing

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in patients, effective amounts for this use will depend on the severity and course of the disease, previous therapy, the patient's health status and response to the drugs, and the judgment of the treating physician. In one aspect, prophylactic treatments include administering to a mammal, who previously experienced at least one symptom of or risk factor for the disease being treated and is currently in remission, a pharmaceutical composition comprising a compound described herein, or a pharmaceutically acceptable salt thereof, in order to prevent a return of the symptoms of the disease or condition.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

In certain embodiments wherein a patient's status does improve, the dose of drug being administered is temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent or daily treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, are from about 0.01 to about 50 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₁₀ and the ED₉₀. The dose ratio between the toxic and therapeutic effects is the therapeutic index and it is expressed as the ratio between LD₁₀ and ED₉₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of the compound, including further embodiments in which (i) the compound is administered once a day; or (ii) the compound is administered to the mammal multiple times over the span of one day.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the mammal every 8 hours; (iv) the compound is administered to the subject every 12 hours; (v) the compound is administered to the subject every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

### Pharmaceutical Compositions/Formulations

The compounds described herein are administered to a subject in need thereof, either alone or in combination with pharmaceutically acceptable carriers, excipients or diluents, in a pharmaceutical composition, according to standard pharmaceutical practice. In one embodiment, the compounds of this invention may be administered to animals. The compounds can be administered orally or parenterally, including the intravenous, intramuscular, intraperitoneal, subcutaneous, rectal and topical routes of administration.

In another aspect, provided herein are pharmaceutical compositions comprising a compound described herein, or a pharmaceutically acceptable salt, solvate, or stereoisomer thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the pharmaceutically acceptable excipient is selected from carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, and any combinations thereof.

The pharmaceutical compositions described herein are administered to a subject by appropriate administration routes, including, but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

Pharmaceutical compositions including compounds described herein, or a pharmaceutically acceptable salt, solvate, or stereoisomer thereof are manufactured in a conventional manner, such as, by way of example only, by means of conventional mixing, dissolving, granulating, dragee-making, levigating, emulsifying, encapsulating, entrapping, or compression processes.

Pharmaceutical compositions for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active compound doses.

Pharmaceutical compositions that are administered orally include push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as talc or magnesium stearate and, optionally, stabilizers. In soft capsules, the active compounds are dissolved or suspended in suitable liquids, such as fatty oils, liquid paraffin, or liquid polyethylene glycols. In some embodiments, stabilizers are added.

Pharmaceutical compositions for parental use are formulated as infusions or injections. In some embodiments, the pharmaceutical composition suitable for injection or infusion includes sterile aqueous solutions, or dispersions, or sterile powders comprising a compound described herein, or a pharmaceutically acceptable salt, solvate, or stereoisomer thereof. In some embodiments, the pharmaceutical composition comprises a liquid carrier. In some embodiments, the liquid carrier is a solvent or liquid dispersion medium comprising, for example, water, saline, ethanol, a polyol (for example, glycerol, propylene glycol, liquid polyethylene glycols, and the like), vegetable oils, nontoxic glyceryl esters, and any combinations thereof. In some embodiments, the pharmaceutical compositions further comprise a preservative to prevent growth of microorganisms.

### Combination

Disclosed herein are method of treating a disease using a compound disclosed herein, or a pharmaceutically acceptable salt, solvate, or stereoisomer thereof, in combination with an additional therapeutic agent. In some embodiments, the additional therapeutic agent is an anti-IL-10 drugs such as anakinra, canakinumab, or rilonacept; an antiviral drugs or nucleoside inhibitors such as remdesivir; colchicine, hydroxychloroquine (antimitotic agents for cancer therapy); gasdermin D inhibitors, for example dimethylfumarate or disulfiram; a multiple sclerosis drug, for example ozanimod, fingolimod, or siponimod; other NLRP3 inhibitors, for example tranilast or dapansutrile; melatonin; an anti-IL-6 agents such as anti-IL-6R antibody tocilizumab (atlizumab), or anti-IL-6 antibody siltuximab; a steroid such as dexamethasone, methyl prednisolone, or prednisone; a diabetes drug, such as metformin, DPP-IV inhibitors (sitagliptin, vildagliptin, saxagliptin, or linagliptin), SGLT inhibitors (dapagliflozin, canaglifozin), pioglitazone, sulfonyl ureas (tolbutamide or glimepiride), glucagon-like peptide agonists (exenatide, liraglutide, or lixasenatide); a gout drug, such as allopurinol or febuxostat; a statin, for example, atorvastatin, rosuvastatin, or pravastatin; a JAK inhibitor such as ruxolitinib; a non-steroidal anti-inflammatory agent, for example ibuprofen, naproxen, celecoxib, indomethacin, diclofenac, aspirin, or a salicylate; or a caspase-1 inhibitor, for example belnacasan (VX-765); or any combinations thereof.

### EXAMPLES

### Example 1. Synthesis of (3-(N-((1,2,3,5,6,7-hexahydro-s-indacen-4-yl)carbamoyl)sulfamoyl)phenyl)boronic acid

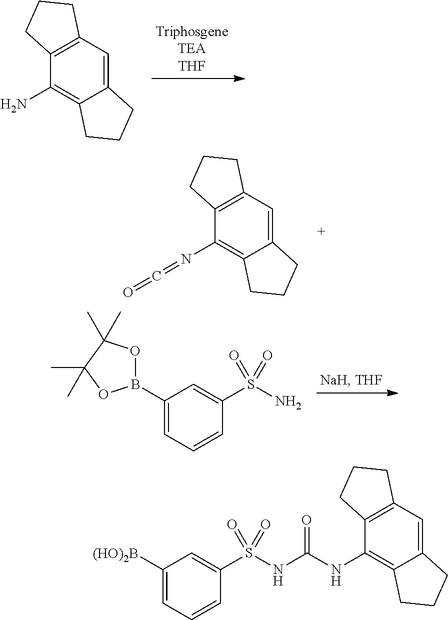

### Step 1: Synthesis of 4-Isocyanato-1,2,3,5,6,7-hexahydro-s-indacene

To a stirred solution of triphosgene (0.36 g, 1.2 mmol) in THF (2 mL) was added the 1,2,3,5,6,7-hexahydro-s-indacen-4-amine (0.24 g, 1.2 mmol) in THF (2 mL) dropwise. Triethylamine (0.37 mL, 24 mmol) in THF (2 mL) was added dropwise. The mixture was stirred at RT for 1 h. The solid was removed by filtration and the filtrate was concentrated in vacuo. The resulting oil was triturated with hexane and the solid was removed by filtration. The filtrate was concentrated in vacuo to give an oil. The isocyanate was used without further purification in the next synthetic step.

### Step 2: Synthesis of (3-(N-((1,2,3,5,6,7-hexahydro-s-indacen-4-yl)carbamoyl)sulfamoyl)phenyl)boronic acid

To a solution of 3-(4,4,5,5-tetramethyl-1,3,2-dioxaborolan-2-yl)benzenesulfonamide (283 mg, 1.0 mmol) in THF (10 mL) at 0° C. was treated with NaH (60 mg, 1.5 mmol, 60% dispersion in oil) and stirred for 15 min. A solution of isocyanate from step 1 (250 mg, 1.2 mmol) in THF (2 mL) was added. The reaction was stirred at RT for 1 h before water was added (10 ml). The aqueous phase was extracted with EtOAc. The organic phase was dried over anhydrous Na₂SO₄ and concentrated. The residue was purified by reverse phase HPLC. Lyophilization of the appropriate fractions afforded the titled compound as white solid. ESI-MS, m/z 401.2 (M+H)⁺.

### Example 2. Synthesis of N-((1,2,3,5,6,7-hexahydro-s-indacen-4-yl)carbamoyl)-3-((3aS,4S,6S,7aR)-3a,5,5-trimethylhexahydro-4,6-methanobenzo[d][1,3,2]dioxaborol-2-yl)benzenesulfonamide

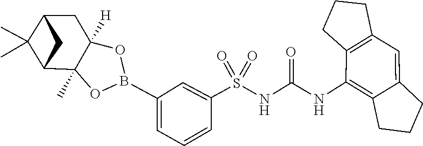 [SMILES: CC1(C)[C@@H]2C[C@H]3OB(c4cccc(S(=O)(=O)NC(=O)Nc5c6c(cc7c5CCC7)CCC6)c4)O[C@@]3(C)[C@H]1C2]

To (3-(N-((1,2,3,5,6,7-hexahydro-s-indacen-4-yl)carbamoyl)sulfamoyl)phenyl)boronic acid (20 mg) in THF (2 mL) was added (1S,2S,3R,5S)-(+)-pinanediol (34 mg) and the resulting solution was stirred at RT overnight. After removal of the solvent, the residue was purified by chromatography on silica gel to afford the titled compound as white solid. ESI-MS, m/z 535.2 (M+H)⁺.

### Example 3. Synthesis of (4-(N-((2,6-diisopropylphenyl)carbamoyl)sulfamoyl)phenyl)boronic acid

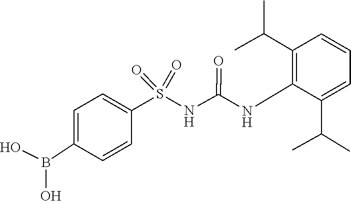 [SMILES: CC(C)c1cccc(C(C)C)c1NC(=O)NS(=O)(=O)c1ccc(B(O)O)cc1]

The title compounds were prepared following the procedure described in Example 1, Step 2 using 2-isocyanato-1,3-diisopropylbenzene. ESI-MS, m/z 405.2 (M+H)⁺.

### Example 4. Synthesis of N-((2,6-diisopropylphenyl)carbamoyl)-4-((3aS,4S,6S,7aR)-3a,5,5-trimethylhexahydro-4,6-methanobenzo[d][1,3,2]dioxaborol-2-yl)benzenesulfonamide

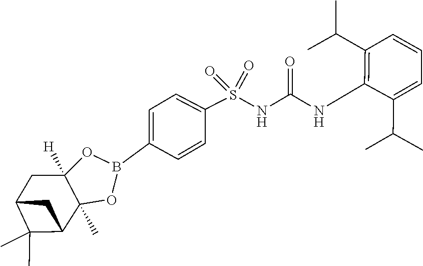 [SMILES: CC(C)c1cccc(C(C)C)c1NC(=O)NS(=O)(=O)c1ccc(B2O[C@@H]3C[C@@H]4C[C@@H](C4(C)C)[C@]3(C)O2)cc1]

The title compounds were prepared following the procedure described in Example 2. ESI-MS, m/z 539.2 (M+H)⁺.

### Example 5. Synthesis of (5-(N-((2,6-diisopropylphenyl)carbamoyl)sulfamoyl)thiophen-2-yl)boronic acid

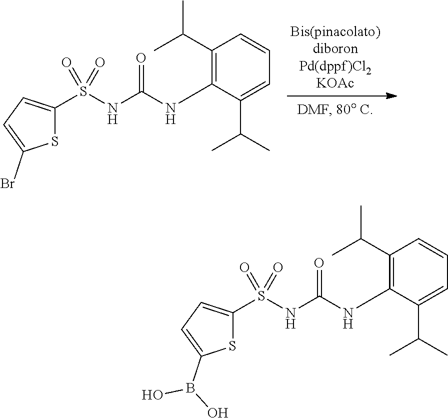 [SMILES: CC(C)c1cccc(C(C)C)c1NC(=O)NS(=O)(=O)c1ccc(Br)s1 ; CC(C)c1cccc(C(C)C)c1NC(=O)NS(=O)(=O)c1ccc(B(O)O)s1]

### Step 1: Synthesis of 5-bromo-N-((2,6-diisopropylphenyl)carbamoyl)thiophene-2-sulfonamide

The title compound was synthesized following the procedure described in Example 1. ESI-MS, m/z 445 (M+H)⁺.

### Step 2: Synthesis of (5-(N-((2,6-diisopropylphenyl)carbamoyl)sulfamoyl)thiophen-2-yl)boronic acid

To a mixture of 5-bromo-N-((2,6-diisopropylphenyl)carbamoyl)thiophene-2-sulfonamide (222 mg, 0.5 mmol), bis(pinacolato)diboron (254 mg, 1 mmol), KOAc (200 mg, 2 mmol), and Pd (dppf)Cl₂ (0.02 mmol) in a vial was added DMF (3 mL). The reaction mixture was stirred at 80° C. for 1 h. Water was added and the aqueous phase was extracted with EtOAc. The organic phase was dried over anhydrous Na₂SO₄ and concentrated. The residue was purified by reverse phase HPLC. Lyophilization of the appropriate fractions afforded the titled compound as off-white solid. ESI-MS, m/z 411.1 (M+H)⁺.

### Example 6. Synthesis of N-((2,6-diisopropylphenyl)carbamoyl)-4-methyl-3-(4,4,5,5-tetramethyl-1,3,2-dioxaborolan-2-yl)benzenesulfonamide

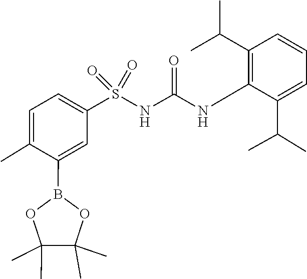 [SMILES: Cc1ccc(S(=O)(=O)NC(=O)Nc2c(C(C)C)cccc2C(C)C)cc1B1OC(C)(C)C(C)(C)O1]

The title compound was prepared following the procedure described in Example 3. ESI-MS, m/z 501.2 (M+H)⁺.

### Example 7. Synthesis of (5-(N-((2,6-diisopropylphenyl)carbamoyl)sulfamoyl)-2-methylphenyl)boronic acid

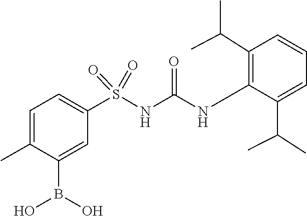 [SMILES: Cc1ccc(S(=O)(=O)NC(=O)Nc2c(C(C)C)cccc2C(C)C)cc1B(O)O]

The title compound was prepared following the procedure described in Example 3. ESI-MS, m/z 419.2 (M+H)⁺.

### Example 8. Synthesis of (3-(N-((2,6-diisopropylphenyl)carbamoyl)sulfamoyl)-4-methoxyphenyl)boronic acid

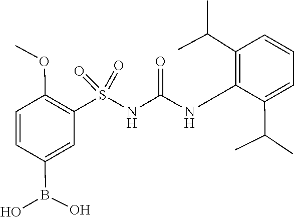 [SMILES: COc1ccc(B(O)O)cc1S(=O)(=O)NC(=O)Nc1c(C(C)C)cccc1C(C)C]

The title compound was prepared following the procedure described in Example 3. ESI-MS, m/z 435.2 (M+H)⁺.

### Example 9. Synthesis of N-((2,6-diisopropylphenyl)carbamoyl)-2-methoxy-5-(4,4,5,5-tetramethyl-1,3,2-dioxaborolan-2-yl)benzenesulfonamide

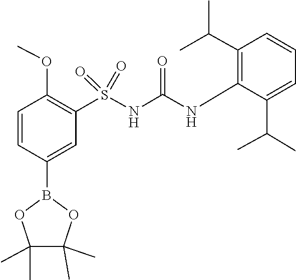 [SMILES: COc1ccc(B2OC(C)(C)C(C)(C)O2)cc1S(=O)(=O)NC(=O)Nc1c(C(C)C)cccc1C(C)C]

The title compound was prepared following the procedure described in Example 3. ESI-MS, m/z 517.2 (M+H)⁺.

### Example 10. Synthesis of (2-(N-((1,2,3,5,6,7-hexahydro-s-indacen-4-yl)carbamoyl)sulfamoyl)phenyl)boronic acid

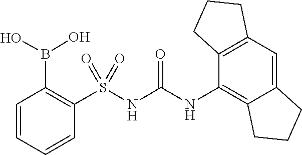 [SMILES: O=C(Nc1c2c(cc3c1CCC3)CCC2)NS(=O)(=O)c1ccccc1B(O)O]

The title compound was prepared following the procedure described in Example 1. ESI-MS, m/z 401.2 (M+H)⁺.

### Example 11. Synthesis of (2-(N-((2,6-diisopropylphenyl)carbamoyl)sulfamoyl)phenyl)boronic acid

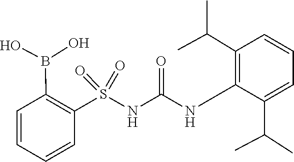 [SMILES: CC(C)c1cccc(C(C)C)c1NC(=O)NS(=O)(=O)c1ccccc1B(O)O]

The title compound was prepared following the procedure described in Example 3. ESI-MS, m/z 405.2 (M+H)⁺.

### Example 12. Synthesis of (5-(N-((1,2,3,5,6,7-hexahydro-s-indacen-4-yl)carbamoyl)sulfamoyl)thiophen-2-yl)boronic acid

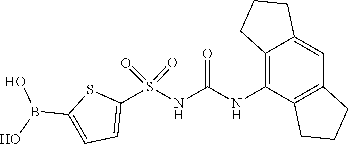 [SMILES: O=C(Nc1c2c(cc3c1CCC3)CCC2)NS(=O)(=O)c1ccc(B(O)O)s1]

The title compound was prepared following the procedure described in Example 5. ESI-MS, m/z 407.1 (M+H)⁺.

### Example 13. Synthesis of (4-(N-((1,2,3,5,6,7-hexahydro-s-indacen-4-yl)carbamoyl)sulfamoyl)phenyl boronic acid

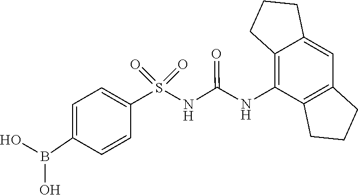 [SMILES: O=C(Nc1c2c(cc3c1CCC3)CCC2)NS(=O)(=O)c1ccc(B(O)O)cc1]

The title compound was prepared following the procedure described in Example 1. ESI-MS, m/z 401.1 (M+H)⁺.

### Example 14. Synthesis of N-((1,2,3,5,6,7-hexahydro-s-indacen-4-yl)carbamoyl)-4-((3aS,4S,6S)-3a,5,5-trimethylhexahydro-4,6-methanobenzo[d][1,3,2]dioxaborol-2-yl)benzenesulfonamide

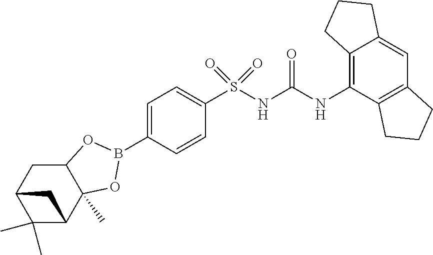 [SMILES: CC1(C)[C@@H]2CC3OB(c4ccc(S(=O)(=O)NC(=O)Nc5c6c(cc7c5CCC7)CCC6)cc4)O[C@@]3(C)[C@H]1C2]

The title compound was prepared following the procedure described in Example 2. ESI-MS, m/z 535.2 (M+H)⁺.

### Example 15. Synthesis of (3-(N-((1,2,3,5,6,7-hexahydro-s-indacen-4-yl)carbamoyl)sulfamoyl)-2-methylphenyl)boronic acid

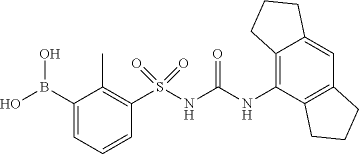 [SMILES: Cc1c(B(O)O)cccc1S(=O)(=O)NC(=O)Nc1c2c(cc3c1CCC3)CCC2]

The title compound was prepared following the procedure described in Example 1. ESI-MS, m/z 415.1 (M+H)⁺.

### Example 16. Synthesis of N-((1,2,3,5,6,7-hexahydro-s-indacen-4-yl)carbamoyl)-2-methyl-3-(4,4,5,5-tetramethyl-1,3,2-dioxaborolan-2-yl)benzenesulfonamide

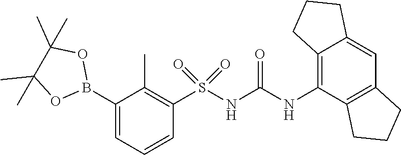 [SMILES: Cc1c(B2OC(C)(C)C(C)(C)O2)cccc1S(=O)(=O)NC(=O)Nc1c2c(cc3c1CCC3)CCC2]

The title compound was prepared following the procedure described in Example 1. ESI-MS, m/z 497.2 (M+H)⁺.

### Example 17. Synthesis of N-((1,2,3,5,6,7-hexahydro-s-indacen-4-yl)carbamoyl)-1-hydroxy-1,3-dihydrobenzo[c][1,2]oxaborole-6-sulfonamide

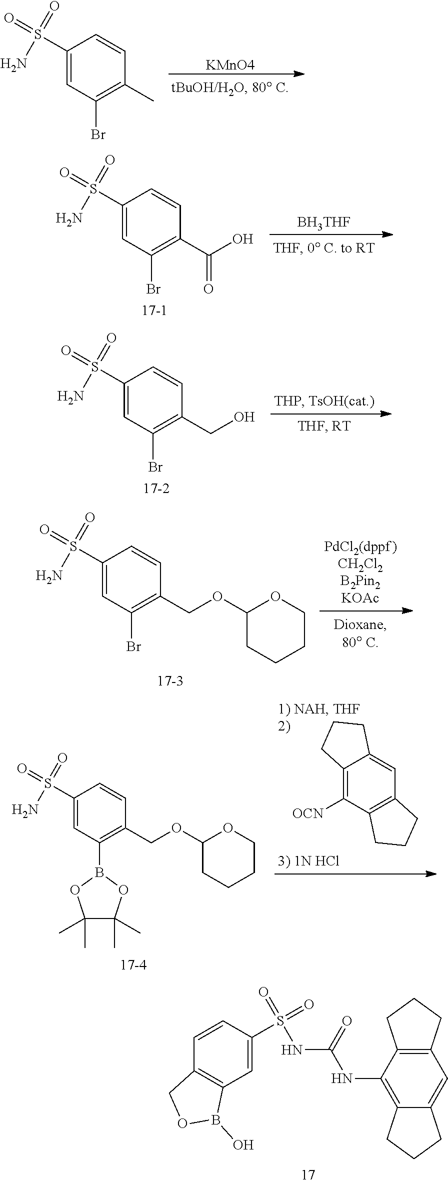

### Step 1. Synthesis of 2-bromo-4-sulfamoylbenzoic acid (17-1)

To a solution of 3-bromo-4-methylbenzenesulfonamide (2.5 g, 10 mmol) in tBuOH (25 mL) and H₂O (25 mL) was added KMnO₄ (4.0 g, 25 mmol) at RT. The resulting reaction mixture was stirred at 80° C. for 6 h. After the reaction mixture was cooled to RT, 1 N HCl was added to adjust the pH to 2. The aqueous phase was extracted with EtOAc, dried over Na₂SO₄, and concentrated to afford 2-bromo-4-sulfamoylbenzoic acid as white solid. ESI-MS, m/z 279.9 (M+H)⁺.

### Step 2. Synthesis of 3-bromo-4-(hydroxymethyl)benzenesulfonamide (17-2)

To a solution of 2-bromo-4-sulfamoylbenzoic acid (1.8 g, 6.5 mmol) in THF (30 mL) at 0° C. was added BH₃-THF (1 M in THF, 20 mL) slowly. The reaction mixture was stirred overnight to let warm up to RT. Sat. NH₄Cl was added, and the aqueous phase was extracted with EtOAc. The combined organic phase was dried over Na₂SO₄, and concentrated to afford 3-bromo-4-(hydroxymethyl)benzenesulfonamide as clear oil. ESI-MS, m/z 265.9 (M+H)⁺.

### Step 3. Synthesis of 3-bromo-4-(((tetrahydro-2H-pyran-2-yl)oxy)methyl)benzenesulfonamide (17-3)

To a solution of 3-bromo-4-(hydroxymethyl)benzenesulfonamide (0.8 g, 3 mmol) in THF (10 mL) was added 3,4-dihydro-2H-pyran (0.5 g, 6 mmol), followed by TsOH (50 mg). The reaction mixture was stirred overnight. The solvent was then removed and the residue was purified by chromatography to afford 3-bromo-4-(((tetrahydro-2H-pyran-2-yl)oxy)methyl)benzenesulfonamide as oil. ESI-MS, m/z 350.1 (M+H)⁺.

### Step 4. Synthesis of 4-(((tetrahydro-2H-pyran-2-yl)oxy)methyl)-3-(4,4,5,5-tetramethyl-1,3,2-dioxaborolan-2-yl)benzenesulfonamide (17-4)

To a flask containing 3-bromo-4-(((tetrahydro-2H-pyran-2-yl)oxy)methyl)benzenesulfonamide (1.2 g, 3.4 mmol), B₂Pin₂ (1.3 g, 5.2 mmol), PdCl₂ (dppf)CH₂Cl₂ (280 mg, 0.34 mmol), was added dioxane (20 mL), followed by KOAc (1.7 g, 17 mmol). The reaction mixture was heated at 80° C. for 6 hrs. The cooled reaction mixture was diluted with EtOAc, washed with sat. NH₄Cl. The organic phase was dried over Na2SO4, and concentrated. The crude product was purified by chromatography to afford 4-(((tetrahydro-2H-pyran-2-yl)oxy)methyl)-3-(4,4,5,5-tetramethyl-1,3,2-dioxaborolan-2-yl)benzenesulfonamide as off-white solid. ESI-MS, m/z 398.1 (M+H)⁺.

### Step 5. Synthesis of N-((1,2,3,5,6,7-hexahydro-s-indacen-4-yl)carbamoyl)-1-hydroxy-1,3-dihydrobenzo[c][1,2]oxaborole-6-sulfonamide (17)

To a solution of 4-(((tetrahydro-2H-pyran-2-yl)oxy)methyl)-3-(4,4,5,5-tetramethyl-1,3,2-dioxaborolan-2-yl)benzenesulfonamide (794 mg, 2.0 mmol) in THF (10 mL) at 0° C. was treated with NaH (160 mg, 4 mmol, 60% dispersion in oil) and stirred for 15 min. A solution of isocyanate (440 mg, 2.2 mmol) in THF (5 mL) was added. The reaction was stirred at RT for 1 h before water was added (10 ml). The aqueous phase was extracted with EtOAc. The organic phase was dried over anhydrous Na₂SO₄ and concentrated. The residue was dissolved in THF (10 mL) and MeOH (10 mL). 1N HCl (10 mL) was added. The reaction mixture was stirred at RT for 1 h. The reaction mixture was diluted with EtOAc and washed with sat. NH₄Cl solution. The organic phase was dried over Na₂SO4, concentrated and purified by reverse phase HPLC to afford the title compound as white solid. ESI-MS, m/z 413.1 (M+H)⁺.

### Example 18. Synthesis of 1-isopropyl-N-((2-isopropyl-4-(4,4,5,5-tetramethyl-1,3,2-dioxaborolan-2-yl)phenyl)carbamoyl)-1H-pyrazole-4-sulfonamide

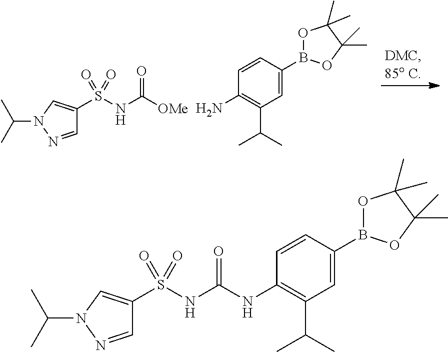 [SMILES: COC(=O)NS(=O)(=O)c1cnn(C(C)C)c1 ; CC(C)c1cc(B2OC(C)(C)C(C)(C)O2)ccc1N ; CC(C)c1cc(B2OC(C)(C)C(C)(C)O2)ccc1NC(=O)NS(=O)(=O)c1cnn(C(C)C)c1]

A solution of methyl ((1-isopropyl-1H-pyrazol-4-yl)sulfonyl)carbamate (123 mg, 0.47 mmol) and 2-isopropyl-4-(4,4,5,5-tetramethyl-1,3,2-dioxaborolan-2-yl)aniline (116 mg, 0.47 mmol) in DMC (3 mL) was heated to 85° C. for 48 h, periodically adding DMC to maintain volume. The reaction mixture was concentrated and the resultant residue was purified via reverse phase chromatography. Lyophilization of the appropriate fractions afforded the title compound as an off-white solid. ESI-MS, m/z 476.8 (M+H)⁺.

### Example 19. Synthesis of (3-isopropyl-4-(3-((1-isopropyl-1H-pyrazol-4-yl)sulfonyl)ureido)phenyl)boronic acid

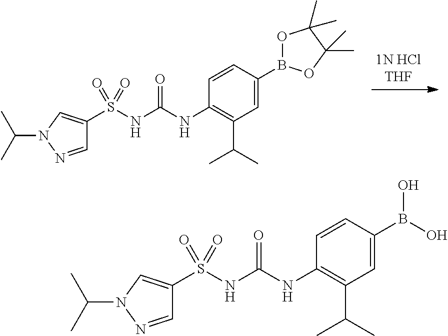

To a solution of 1-isopropyl-N-((2-isopropyl-4-(4,4,5,5-tetramethyl-1,3,2-dioxaborolan-2-yl)phenyl)carbamoyl)-1H-pyrazole-4-sulfonamide (125 mg) in THF (3 mL) was added 1 N HCl (1 mL). After 3 h the reaction mixture was concentrated, and the resultant residue was purified via reverse phase HPLC. Lyophilization of the appropriate fractions afforded the title compound as an off-white solid. ESI-MS, m/z 394.8 (M+H)⁺.

### Example 20. Synthesis of N-((2-cyclopropyl-5-(4,4,5,5-tetramethyl-1,3,2-dioxaborolan-2-yl)phenyl)carbamoyl)-1-isopropyl-1H-pyrazole-4-sulfonamide

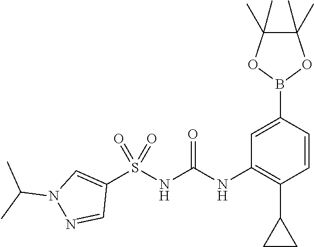 [SMILES: CC(C)n1cc(S(=O)(=O)NC(=O)Nc2cc(B3OC(C)(C)C(C)(C)O3)ccc2C2CC2)cn1]

The title compound was prepared following the procedure described in Example 18, using 2-cyclopropyl-4-(4,4,5,5-tetramethyl-1,3,2-dioxaborolan-2-yl)aniline. ESI-MS, m/z 474.7 (M+H)⁺.

### Example 21. Synthesis of (4-cyclopropyl-3-(3-((1-isopropyl-1H-pyrazol-4-yl)sulfonyl)ureido)phenyl)boronic acid

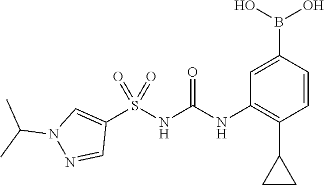 [SMILES: CC(C)n1cc(S(=O)(=O)NC(=O)Nc2cc(B(O)O)ccc2C2CC2)cn1]

The title compound was prepared following the procedure described in Example 19. ESI-MS, m/z 392.8 (M+H)⁺.

### Example 22. Synthesis of N-((2-(tert-butyl)-5-(4,4,5,5-tetramethyl-1,3,2-dioxaborolan-2-yl)phenyl)carbamoyl)-1-isopropyl-1H-pyrazole-4-sulfonamide

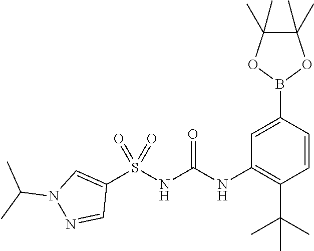 [SMILES: CC(C)n1cc(S(=O)(=O)NC(=O)Nc2cc(B3OC(C)(C)C(C)(C)O3)ccc2C(C)(C)C)cn1]

The title compound was prepared following the procedure described in Example 18, using 2-(tert-butyl)-4-(4,4,5,5-tetramethyl-1,3,2-dioxaborolan-2-yl)aniline. ESI-MS, m/z 490.8 (M+H)⁺.

### Example 23. Synthesis of (4-(tert-butyl)-3-(3-((1-isopropyl-1H-pyrazol-4-yl)sulfonyl)ureido)phenyl)boronic acid

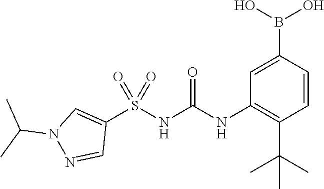 [SMILES: CC(C)n1cc(S(=O)(=O)NC(=O)Nc2cc(B(O)O)ccc2C(C)(C)C)cn1]

The title compound was prepared following the procedure described in Example 19. ESI-MS, m/z 408.8 (M+H)⁺.

### Example 24. Synthesis of (3,5-diisopropyl-4-(3-((1-isopropyl-1H-pyrazol-4-yl)sulfonyl)ureido)phenyl)boronic acid

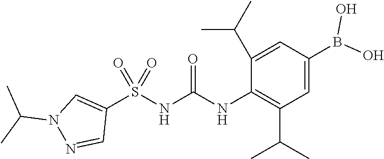 [SMILES: CC(C)c1cc(B(O)O)cc(C(C)C)c1NC(=O)NS(=O)(=O)c1cnn(C(C)C)c1]

The title compound was prepared following the procedure described in Example 1, Steps 1 and 2, using 1-isopropyl-1H-pyrazole-2-sulfonamide and 2,6-diisopropyl-4-(4,4,5,5-tetramethyl-1,3,2-dioxaborolan-2-yl)aniline. ESI-MS, m/z 436.8 (M)⁺.

### Example 25. Synthesis of (8-(3-((1-isopropyl-1H-pyrazol-3-yl)sulfonyl)ureido)-1,2,3,5,6,7-hexahydro-s-indacen-4-yl)boronic acid

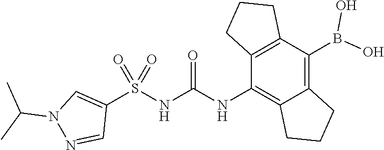 [SMILES: CC(C)n1cc(S(=O)(=O)NC(=O)Nc2c3c(c(B(O)O)c4c2CCC4)CCC3)cn1]

The title compound was prepared following the procedure described in Example 1, Steps 1 and 2, using 1-isopropyl-1H-pyrazole-2-sulfonamide and 8-(4,4,5,5-tetramethyl-1,3,2-dioxaborolan-2-yl)-1,2,3,5,6,7-hexahydro-s-indacen-4-amine. ESI-MS, m/z 454.8 (M+Na)⁺.

### Example 26. Synthesis of N-((1,2,3,5,6,7-hexahydro-s-indacen-4-yl)carbamoyl)-3-(4-methyl-2,6-dioxotetrahydro-2H-414,814-[1,3,2]oxazaborolo[2,3-b][1,3,2]oxazaborol-8-yl)benzenesulfonamide

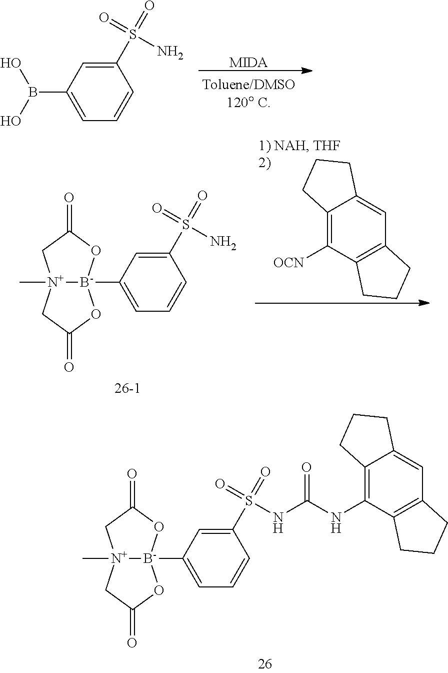

### Step 1. Synthesis of (3-sulfamoylphenyl)boronic acid MIDA ester (26-1)

A mixture of (3-sulfamoylphenyl)boronic acid (400 mg, 2 mmol) and MIDA (420 mg, 3 mmol) in Toluene and DMSO (20 mL/20 mL) was heated at 120° C. for 2 h. Water was then added, and the reaction mixture was extracted with EtOAc. The organic phase was dried over Na₂SO₄ and concentrated. The residue was dissolved in 5 mL of MeOH and ethyl ether was added to precipitate out the product, which was filtered and dried to afford (3-sulfamoylphenyl)boronic acid MIDA ester.

### Step 2. Synthesis of (3-(N-((1,2,3,5,6,7-hexahydro-s-indacen-4-yl)carbamoyl)sulfamoyl)phenyl)boronic acid MIDA ester

The title compound was prepared following the procedure described in Example 1, Step 2. ESI-MS, m/z 512.2 (M+H)⁺.

### Example 27. Synthesis of N-((1,2,3,5,6,7-hexahydro-s-indacen-4-yl)carbamoyl)-4-(4-methyl-2,6-dioxotetrahydro-2H-414,814-[1,3,2]oxazaborolo[2,3-b][1,3,2]oxazaborol-8-yl)benzenesulfonamide

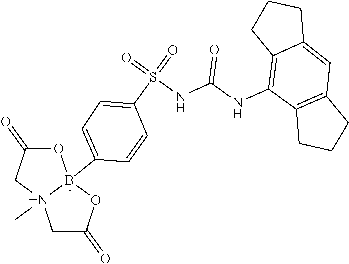

The title compound was prepared following the procedure described in Example 26. ESI-MS, m/z 512.2 (M+H)⁺.

### Example 28. Synthesis of N-((1,2,3,5,6,7-hexahydro-s-indacen-4-yl)carbamoyl)-3-(4-methyltetrahydro-2H-414,814-[1,3,2]oxazaborolo[2,3-b][1,3,2,]oxazaborol-8-yl)benzenesulfonamide

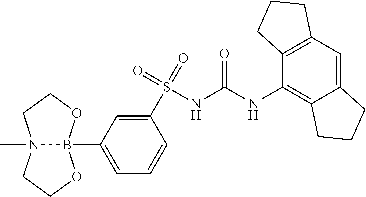

A mixture of N-((1,2,3,5,6,7-hexahydro-s-indacen-4-yl)carbamoyl)-3-(4,4,5,5-tetramethyl-1,3,2-dioxaborolan-2-yl)benzenesulfonamide (20 mg, 0.04 mmol) and N-methyldiethanolamine (12 mg) in acetone (1.5 mL) was stirred at RT for 20 h. Diluted with Et₂O (3 mL), collected the solid and dried to give 28 as white solid: ESI-MS, m/z 401.1.

### Example 29. Synthesis of N-((1,2,3,5,6,7-hexahydro-s-indacen-4-yl)carbamoyl)-1-hydroxy-1,3-dihydrobenzo[c][1,2]oxaborole-5-sulfonamide

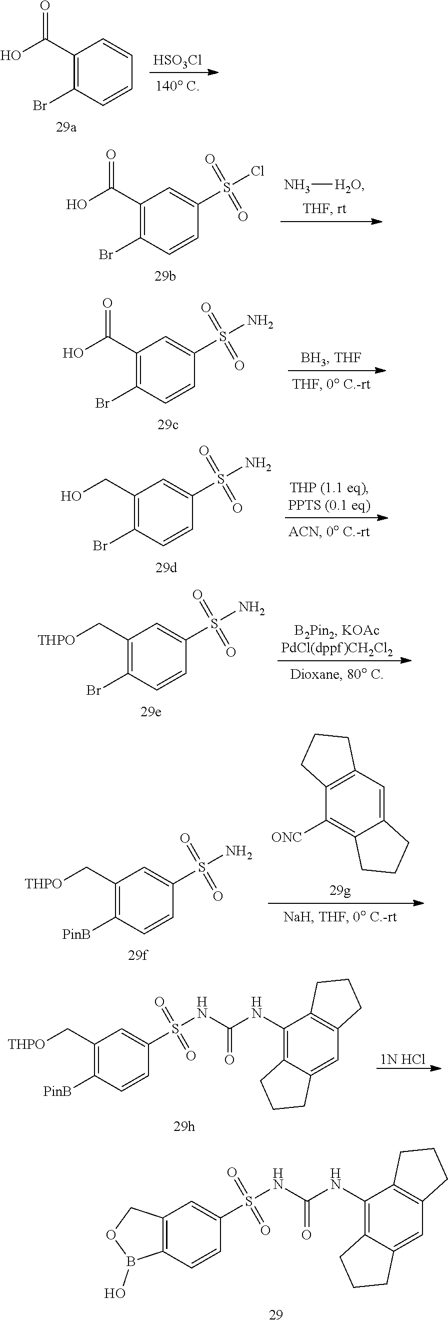

### Step 1: Synthesis of 2-bromo-5-(chlorosulfonyl)benzoic acid (29b)

To a solution of 29a (300 g, 1500 mmol) was added HSO₃Cl (1500 mL) dropwise, and then the resulting mixture was heated at 140° C. for 3 h. The resulting mixture was cooled to rt, and then poured into ice water, filtered and the solid was dried by air to afford the product as yellow solid (350 g). Yield 80%. ESI-MS, m/z 300.1 (M+H)⁺.

### Step 2: Synthesis of 2-bromo-5-sulfamoylbenzoic acid (29c)

A solution of 29b (150 g, 500 mmol) in THF (300 mL) was added dropwise slowly to NH₃·H₂O (500 mL) at 0° C., and then the resulting mixture was warmed to RT for 2 h. Concentrated to afford the product as yellow solid (130 g). Yield 92%. ESI-MS, m/z 282.0 (M+H)⁺.

### Step 3: Synthesis of 4-bromo-3-(hydroxymethyl)benzenesulfonamide (29d)

To a solution of 29c (100 g, 358 mmol) in THF (200 mL) was added BH₃ in THF (500 mL) slowly at 0° C., and then the resulting mixture was warmed to RT for 16 h. The reaction was quenched by MeOH (50 mL) (be careful of gas), concentrated and the residue was washed by EA (500 mL). Concentrated to afford the product as white solid (70 g). Yield 82%. ESI-MS, m/z 266.1 (M+H)⁺.

### Step 4: Synthesis of 4-bromo-3-(((tetrahydro-2H-pyran-2-yl)oxy)methyl)benzenesulfonamide (29e)

To a solution of 29d (26.5 g, 100 mmol) in ACN (200 mL), THP (9.5 g, 110 mmol) and PPTS (2.5 g, 10 mmol) were added at 0° C., and then the resulting mixture was warmed to RT for 16 h. The reaction was washed with brine (2×50 mL). The organic layer was dried over Na₂SO₄, concentrated, and purified by FCC (PE:EA=25%) to afford the product as white solid (13.4 g). Yield 38%. ESI-MS, m/z 372.1 (M+Na)⁺.

### Step 5: Synthesis of 3-(((tetrahydro-2H-pyran-2-yl)oxy)methyl)-4-(4,4,5,5-tetramethyl-1,3,2-dioxaborolan-2-yl)benzenesulfonamide (29f)

To a solution of 29e (13.4 g, 50 mmol) in dioxane (100 mL), B₂Pin₂ (9.5 g, 110 mmol), Pd(dppf)Cl₂·CH₂Cl₂ (2 g, 2.5 mmol), and KOAc (9.8 g, 100 mmol) were added at rt. And then the resulting mixture was stirred at 80° C. under nitrogen for 16 h. The mixture was concentrated and purified by FCC (PE:EA=20%) to afford the product as white solid (11.6 g). Yield 76%. ESI-MS, m/z 420.1 (M+Na)⁺.

### Step 6: Synthesis of 3-(((tetrahydro-2H-pyran-2-yl)oxy)methyl)-4-(4,4,5,5-tetramethyl-1,3,2-dioxaborolan-2-yl)benzenesulfonamide (29h)

To a solution of 29f (397 mg, 1 mmol) in THF (10 mL), NaH (240 mg, 10 mmol) was added at 0° C. under nitrogen. After 20 min, 29g (200 mg, 1 mmol) was added. And then the resulting mixture was stirred at RT for 16 h. The mixture was quenched by aq. NH₄Cl (5 mL) to afford the product as white solid (650 mg crude) which directly used for next step without further purification. ESI-MS, m/z 619.0 (M+Na)⁺.

### Step 7: Synthesis of n-((1,2,3,5,6,7-hexahydro-s-indacen-4-yl)carbamoyl)-1-hydroxy-1,3-dihydrobenzo[c][1,2]oxaborole-5-sulfonamide (29)

To a solution of 29h (650 mg, 1 mmol) in THF (10 mL) was added 1 N HCl (5 mL) at 0° C. And then the resulting mixture was stirred at RT for 4 h. The mixture was extracted with EA (3×15 mL), the water layer was dried by freeze-drying. The residue was purified by prep-HPLC (ACN:H₂O:NH₃—H₂O (0.05%), 10% to 40%) to afford the product as white solid (68.5 mg). Yield: 16%. ESI-MS, m/z 412.8 (M+H)⁺.

### Example 30. Synthesis of N-((2,6-diisopropyl-4-(4,4,5,5-tetramethyl-1,3,2-dioxaborolan-2-yl)phenyl)carbamoyl)-1-isopropyl-1H-pyrazole-3-sulfonamide

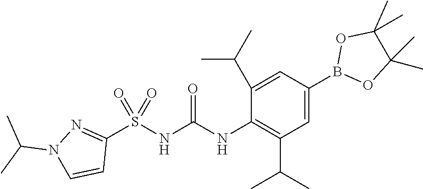 [SMILES: CC(C)c1cc(B2OC(C)(C)C(C)(C)O2)cc(C(C)C)c1NC(=O)NS(=O)(=O)c1ccn(C(C)C)n1]

The title compound was prepared following the procedure described in Example 1, Step 1 and Example 1, Step 2, using 1-isopropyl-1H-pyrazole-3-sulfonamide and 2,6-diisopropyl-4-(4,4,5,5-tetramethyl-1,3,2-dioxaborolan-2-yl)aniline. ESI-MS, m/z 417.3/504.3 (M−101)⁺.

### Example 31. Synthesis of (3,5-diisopropyl-4-(3-((1-isopropyl-1H-pyrazol-3-yl)sulfonyl)ureido)phenyl)boronic acid

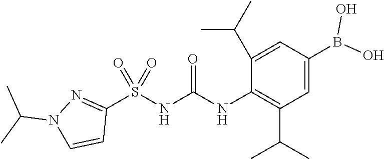 [SMILES: CC(C)c1cc(B(O)O)cc(C(C)C)c1NC(=O)NS(=O)(=O)c1ccn(C(C)C)n1]

The title compound was prepared following the procedure described in Example 19 using N-((2,6-diisopropyl-4-(4,4,5,5-tetramethyl-1,3,2-dioxaborolan-2-yl)phenyl)carbamoyl)-1-isopropyl-1H-pyrazole-3-sulfonamide as the starting material. ESI-MS, m/z 437.2 (M+H)⁺.

### Example 32. Synthesis of N-((1,2,3,5,6,7-hexahydro-s-indacen-4-yl)carbamoyl)-4-methoxy-3-(2-(4,4,5,5-tetramethyl-1,3,2-dioxaborolan-2-yl)ethyl)benzenesulfonamide

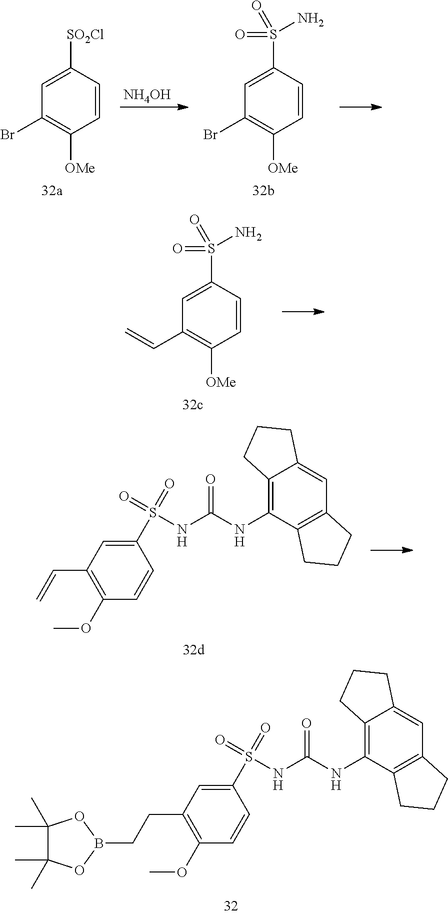

### Step 1: Synthesis of 3-bromo-4-methoxybenzenesulfonamide (32b)

NH₄₀H (28% aq, 5 mL) was added dropwise to a solution of 32a (5 g, 17.5 mmol) in CH₃CN at 5° C. After 20 min., the reaction mixture was warmed to RT for 1 h, then, concentrated. The residue was triturated with water, filtered, and dried to give 32b as white solid: ESI-MS, m/z 266.0/268.0.

### Step 2: Synthesis of 4-methoxy-3-vinylbenzenesulfonamide (32c)

A mixture of 32b (0.5 g, 1.9 mmol), potassium vinyltrifluoroborate (0.3 g, 2.2 mmol), Pd(Ph₃P)₂Cl₂ (0.1 g), and triethylamine (0.4 g, 4 mmol) in i-PrOH (15 mL) was flushed with argon, then, stirred at 90° C. for 20 hrs. The reaction mixture was diluted with EtOAc and washed with sat. NH₄Cl. The organic phase was dried over Na₂SO₄, concentrated, and purified by flash chromatography (EtOAc/hexanes 0˜ 100%) to afford 32c as off-white solid (0.3 g). ESI-MS, m/z 214.1 (M+H)⁺.

### Step 3: Synthesis of N-((1,2,3,5,6,7-hexahydro-s-indacen-4-yl)carbamoyl)-4-methoxy-3-vinylbenzenesulfonamide (32d)

4-isocyanato-1,2,3,5,6,7-hexahydro-s-indacene (32e, 0.2 g in THF 1 mL, 1 mmol) was added to a mixture of ^{t}-BuOK (0.1 g, 0.8 mmol) and 32c (0.2 g, 0.9 mmol) in THF (2 mL) at 0° C. The mixture was stirred at RT for 2 h. The reaction mixture was quenched with sat. NH₄Cl and extracted with EtOAc. The organic layer was concentrated. The residue was triturated with DCM (4 mL), filtered and dried to afford 32d (0.12 g): ESI-MS, m/z 413.1.

### Step 4: Synthesis of N-((1,2,3,5,6,7-hexahydro-s-indacen-4-yl)carbamoyl)-4-methoxy-3-(2-(4,4,5,5-tetramethyl-1,3,2-dioxaborolan-2-yl)ethyl)benzenesulfonamide

A mixture of 32d (50 mg, 0.12 mmol), dppe (20 mg), and [IrCl(COD)]₂ (20 mg) in DCM (1.5 mL) was flushed with argon, followed by the addition of HBpin (25 mg, 0.2 mmol). The reaction mixture was stirred at RT for 20 h, then, quenched with sat. NH₄Cl and extracted with EtOAc. The organic layer was concentrated and purified by reverse phase HPLC. Lyophilization of the appropriate fractions afforded the title compound as white solid: ESI-MS, m/z 541.2 (M+H)⁺.

### Example 33. Synthesis of N-((1,2,3,5,6,7-hexahydro-s-indacen-4-yl)carbamoyl)-4-(4-methyl-2,6-dioxotetrahydro-2H-414,814-[1,3,2]oxazaborolo[2,3-b][1,3,2]oxazaborol-8-yl)benzenesulfonamide

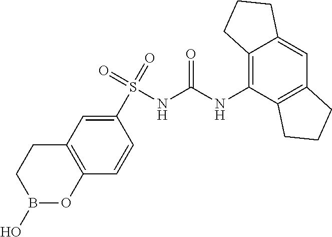 [SMILES: O=C(Nc1c2c(cc3c1CCC3)CCC2)NS(=O)(=O)c1ccc2c(c1)CCB(O)O2]

BBr₃ (1 N, 1 mL) was added dropwise to a solution of 32 (15 mg, 0.03 mmol) in DCM (1 mL) under argon at −70° C. After 1 hr, the reaction mixture was warmed to 0° C. for 1 h. The reaction was quenched with 1 mL 10% citric acid at 0° C. for 20 min., then, extracted with EtOAc. The organic layer was concentrated and purified by reverse phase HPLC. Lyophilization of the appropriate fractions afforded the title compound as white solid. ESI-MS, m/z 427.0 (M+H)⁺.

### Example 34. Synthesis of 5-fluoro-N-((1,2,3,5,6,7-hexahydro-s-indacen-4-yl)carbamoyl)-1-hydroxy-1,3-dihydrobenzo[c][1,2]oxaborole-7-sulfonamide

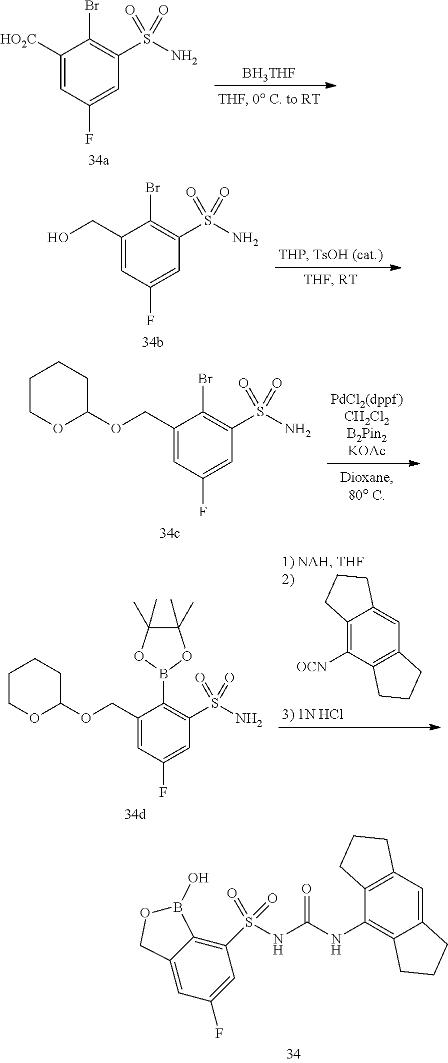

### Step 1: Synthesis of 2-bromo-5-fluoro-3-(hydroxymethyl)benzenesulfonamide (34b)

To a solution of 2-bromo-5-fluoro-3-sulfamoylbenzoic acid 34a (1 g, 3.36 mmol) in THF (17 mL) at 0° C. was added a solution of BH₃-THF (10.4 mL, 1 M/THF). The mixture was then warmed to ambient temperature. After 3 h the reaction mixture was quenched slowly with NH₄Cl (sat) and extracted with EtOAc. The combined organic layers were washed with NH₄Cl (sat) and brine, dried over Na₂SO₄, filtered and concentrated to dryness to afford the titled compound as a white solid which was used without further purification. ESI-MS, m/z 283.9/286.0 (M+H)+

### Step 2: Synthesis of 2-bromo-5-fluoro-3-(((tetrahydro-2H-pyran-2-yl)oxy)methyl)benzenesulfonamide (34c)

To a solution of 34b (500 mg, 1.76 mmol) in THF (15 mL) at ambient temperature were added PTSA (catalytic) and dihydropyran (193 μL, 2.11 mmol). After 4 h the reaction mixture was diluted with EtOAc and washed with NaHCO₃ (sat) and brine. The combined organics were washed over Na₂SO₄, filtered and evaporated to dryness. The crude residue was purified via flash chromatography (SiO₂, 5-100% EtOAc/hexanes) to afford the title compound as a colorless oil. ESI-MS, m/z 368.0/369.9 (M+H)+

### Step 3: Synthesis of 5-fluoro-3-(((tetrahydro-2H-pyran-2-yl)oxy)methyl)-2-(4,4,5,5-tetramethyl-1,3,2-dioxaborolan-2-yl)benzenesulfonamide (34d)

A mixture of 34c (604 mg, 1.64 mmol), B₂Pin₂ (500 mg, 1.97 mmol), and KOAc (483 mg, 4.92 mmol) in dioxane (10 mL) was sparged with Ar. Pd₂Cl₂(dppf) (60 mg, 00.082 mmol) was added and the mixture was heated to 80° C. After 3 h the reaction mixture was cooled to ambient temperature and filtered through celite, washing the Pad with EtOAc. The mixture was further diluted with EtOAc and washed with NH₄Cl (sat) and brine, then dried over Na₂SO₄, filtered and evaporated to dryness. The crude residue was purified via flash chromatography (SiO₂, 5-100% EtOAc/hexanes) to afford the titled compound as a colorless oil. ESI-MS, m/z 416.1 (M+H)+

### Step 4: Synthesis of 5-fluoro-N-((1,2,3,5,6,7-hexahydro-s-indacen-4-yl)carbamoyl)-1-hydroxy-1,3-dihydrobenzo[c][1,2]oxaborole-7-sulfonamide (34)

To a solution of 34d (274 mg, 0.66 mmol) in THF (5 mL) at 0° C. was added NaH (40 mg, 1 mmol, 60% dispersion/mineral oil) and the mixture was warmed to ambient temperature. After 30 min the mixture was cooled to 0° C. and a solution of 4-isocyanato-1,2,3,5,6,7-hexahydro-s-indacene 34e (137 mg, 0.66 mmol) in THF (5 mL) was added over 10 min. The reaction mixture was then allowed to warm slowly to ambient temperature. After 16 h the reaction mixture was diluted with EtOAc and washed with NH₄Cl (sat), NaHCO₃ (sat) and brine. The organic layer was then dried over Na₂SO₄, filtered and evaporated to dryness. The residue was dissolved in THF (2 mL) and MeOH (2 mL), then 1 N HCl (1 mL) was added. After 1 h the reaction mixture was concentrated to dryness and purified via reverse phase HPLC eluting with ACN/water+0.1% TFA. The product-containing fractions were lyophilized to afford the titled compound as a white solid. ESI-MS, m/z 431.1 (M+H)+

### Example 35. Synthesis of (5-(N-((1,2,3,5,6,7-hexahydro-s-indacen-4-yl)carbamoyl)sulfamoyl)-2-(methoxycarbonyl)phenyl)boronic acid

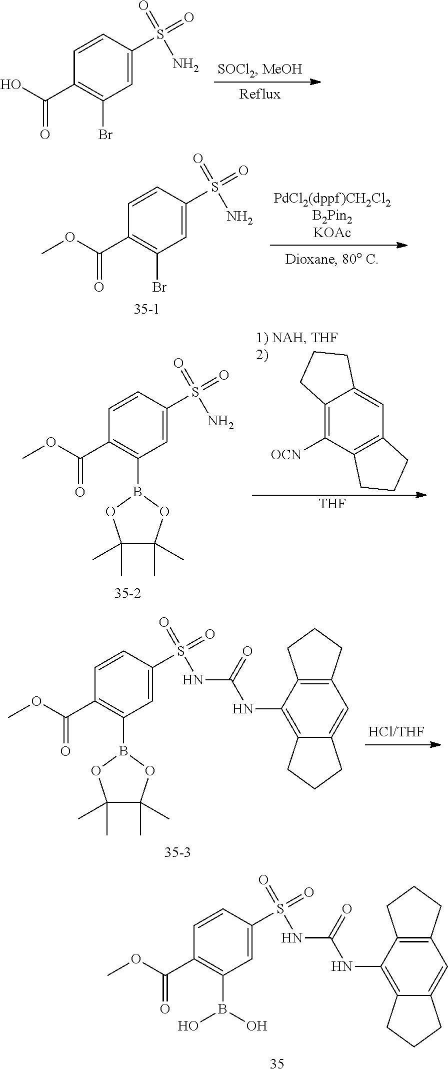

### Step 1. Synthesis of methyl 2-bromo-4-sulfamoylbenzoate (35-1)

Thionyl chloride (5 mL) was added to a solution of 2-bromo-4-sulfamoylbenzoic acid (4.8 g) in methanol (100 mL). The resulting mixture was heated at 70° C. for 12 h and then evaporated to dryness under vacuum. The residue was redissolved in DCM and washed with water and sodium bicarbonate solution. The organic phase was dried over Na₂SO₄, filtered and evaporated to dryness to afford the titled compounds as white solid.

### Step 2. Synthesis of methyl 4-sulfamoyl-2-(4,4,5,5-tetramethyl-1,3,2-dioxaborolan-2-yl)benzoate (35-2)

To a mixture of compound 35-1 (584 mg, 2 mmol), B₂Pin₂ (762 mg, 3 mmol), Pd(dppf) Cl₂·CH₂Cl₂ (163 mg, 0.2 mmol) and KOAc (980 mg, 10 mmol) was added dioxane (20 mL). The resulting reaction mixture as stirred at 80° C. for 12 h. The reaction mixture was cooled to room temperature and diluted with EtOAc, washed with water and brine. The organic phase was dried over Na₂SO₄, and concentrated. The crude product was purified by chromatography to afford the titled compound as solid. ESI-MS, m/z 342.1 (M+H)+

### Step 3. Synthesis of methyl 4-(N-((1,2,3,5,6,7-hexahydro-s-indacen-4-yl)carbamoyl)sulfamoyl)-2-(4,4,5,5-tetramethyl-1,3,2-dioxaborolan-2-yl)benzoate (35-3)

The title compound was prepared following the procedures described in Example 1, Step 1. ESI-MS, m/z 541.2 (M+H)⁺.

### Step 4. Synthesis of (5-(N-((1,2,3,5,6,7-hexahydro-s-indacen-4-yl)carbamoyl)sulfamoyl)-2-(methoxycarbonyl)phenyl)boronic acid (35)

To Compound 35-3 (20 mg) in THF (5 mL) was added 1 N HCl (1 mL) and the resulting reaction mixture was stirred at RT for 1 h. After removal of the solvents, the residue was purified using R-HPLC to afford the titled compound as white solid. ESI-MS, m/z 459.2 (M+H)⁺.

### Example 36. Synthesis of (5-(N-((1,2,3,5,6,7-hexahydro-s-indacen-4-yl)carbamoyl)sulfamoyl)-2-(methoxycarbonyl)phenyl)boronic acid

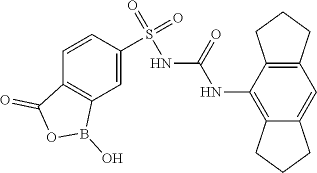 [SMILES: O=C(Nc1c2c(cc3c1CCC3)CCC2)NS(=O)(=O)c1ccc2c(c1)B(O)OC2=O]

To compound 35-3 (50 mg) in MeOH (10 mL) was added 1 N NaOH (1 mL) and the reaction mixture was stirred at RT for 2 h. 1 N aqueous HCl (3 mL) was then added and the reaction mixture was stirred for 1 h. The EtOAc was added to the reaction mixture and the organic phase washed with water and brine, dried over Na₂SO₄ and concentrated. The residue was purified using RP-HPLC to afford the titled compound. m/z 427.2 (M+H)⁺.

### Example 37. Synthesis of (3-(5-(N-((1,2,3,5,6,7-hexahydro-s-indacen-4-yl)carbamoyl)sulfamoyl)-2-hydroxyphenyl)propyl)boronic acid

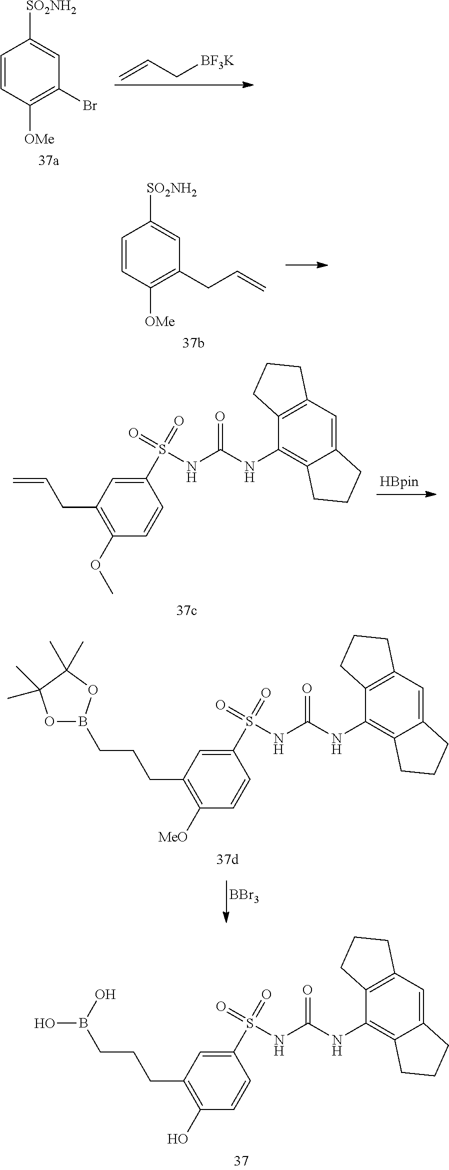

### Step 1: Synthesis of 3-allyl-4-methoxybenzenesulfonamide (37b)

A mixture of 37a (0.5 g, 1.9 mmol), potassium allyltrifluoroborate (0.56 g, 3.8 mmol), Cs₂CO₃ (1.3 g, 3.8 mmol) and Pd(dppf)Cl₂ (200 mg) in 1,4-dioxane (20 mL)/water (2 mL) was flushed with argon, then, heated under microwave reactor at 150° C. for 40 min. After cooling to rt, the reaction mixture was diluted with EtOAc, washed with sat. NH₄Cl. The organic phase was dried over Na₂SO₄, concentrated, and purified by flash chromatography (EtOAc/hexanes 0-100%) to afford 37b as yellow solid (0.2 g): ESI-MS, m/z 250.1 (M+Na)⁺.

### Step 2: Synthesis of 3-allyl-N-((1,2,3,5,6,7-hexahydro-s-indacen-4-yl)carbamoyl)-4-methoxybenzenesulfonamide (37c)

The title compound was prepared following the procedure described in Example 32, Step 3 using 37b: ESI-MS, m/z 427.2 (M+H)⁺.

### Step 3: Synthesis of N-((1,2,3,5,6,7-hexahydro-s-indacen-4-yl)carbamoyl)-4-methoxy-3-(3-(4,4,5,5-tetramethyl-1,3,2-dioxaborolan-2-yl)propyl)benzenesulfonamide (37d)

The title compound was prepared following the procedure described in Example 32, Step 4 using 37c: ESI-MS, m/z 555.3 (M+H)⁺.

### Step 4: Synthesis of (3-(5-(N-((1,2,3,5,6,7-hexahydro-s-indacen-4-yl)carbamoyl)sulfamoyl)-2-hydroxyphenyl)propyl)boronic acid

The title compound was prepared following the procedure described in Example 33 using 37d: ESI-MS, m/z 459.1 (M+H)⁺.

### Example 38 and 39. Synthesis of (5-(N-((1,2,3,5,6,7-hexahydro-s-indacen-4-yl)carbamoyl)sulfamoyl)-2-hydroxybenzyl)boronic acid (38) and (5-(N-((1,2,3,5,6,7-hexahydro-s-indacen-4-yl)carbamoyl)sulfamoyl)-2-methoxybenzyl)boronic acid (39)

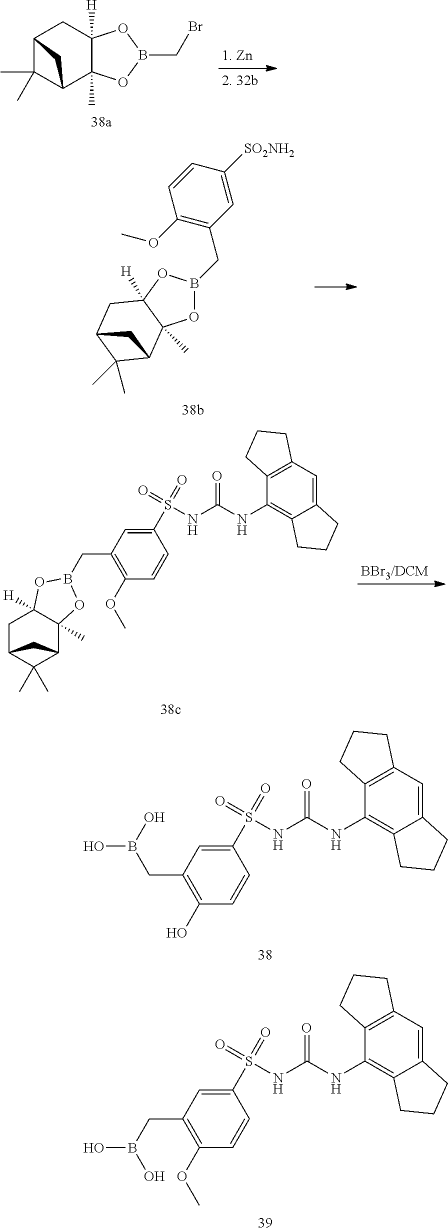 [SMILES: CC1(C)[C@@H]2C[C@H]3OB(CBr)O[C@@]3(C)[C@H]1C2 ; COc1ccc(S(N)(=O)=O)cc1CB1O[C@@H]2C[C@@H]3C[C@@H](C3(C)C)[C@]2(C)O1 ; COc1ccc(S(=O)(=O)NC(=O)Nc2c3c(cc4c2CCC4)CCC3)cc1CB1O[C@@H]2C[C@@H]3C[C@@H](C3(C)C)[C@]2(C)O1 ; O=C(Nc1c2c(cc3c1CCC3)CCC2)NS(=O)(=O)c1ccc(O)c(CB(O)O)c1 ; COc1ccc(S(=O)(=O)NC(=O)Nc2c3c(cc4c2CCC4)CCC3)cc1CB(O)O]

### Step 1: Synthesis of 4-methoxy-3-(((3aS,4S,6S,7aR)-3a,5,5-trimethylhexahydro-4,6-methanobenzo[d][1,3,2]dioxaborol-2-yl)methyl)benzenesulfonamide (38b)

DIBAL (0.3 mL, 1 N in DCM) was added to a mixture of Zn (dust, 0.2 g, 3 mmol) and 38a (0.1 g, 0.36 mmol) in THF (6 mL) under argon at rt. After 10 min, a solution of 38a (0.5 g, 1.8 mmol) in THF (4 mL) was added. The reaction mixture was warmed to 50° C. for 1 h. After cooling to rt, the up-layer clear solution was transferred to a mixture of 32b (0.5 g, 1.9 mmol) and (^{t}Bu₃P)₂Pd (50 mg) in THF (6 mL) under argon. The mixture was stirred at RT for 2 days. The reaction was quenched with sat. NH₄Cl solution and extracted with EtOAc. The organic layer was washed with brine, concentrated and purified by chromatography (EtOAc/Hexanes 0˜ 60%) to afford 38b as colorless oil (0.5 g): ESI-MS, m/z 380.3 (M+H)⁺.

### Step 2: Synthesis of N-((1,2,3,5,6,7-hexahydro-s-indacen-4-yl)carbamoyl)-4-methoxy-3-(((3aS,4S,6S,7aR)-3a,5,5-trimethylhexahydro-4,6-methanobenzo[d][1,3,2]dioxaborol-2-yl)methyl)benzenesulfonamide (38c)

The title compound was prepared following the procedure described in Example 32, Step 3, using 38b: ESI-MS, m/z 579.3 (M+H)⁺.

### Step 3. Synthesis of (5-(N-((1,2,3,5,6,7-hexahydro-s-indacen-4-yl)carbamoyl)sulfamoyl)-2-hydroxybenzyl)boronic acid (38) and (5-(N-((1,2,3,5,6,7-hexahydro-s-indacen-4-yl)carbamoyl)sulfamoyl)-2-methoxybenzyl)boronic acid (39)

The title compound was prepared following the procedure described in Example 33 using BBr₃/DCM and 38c, after reverse phase HPLC purification to afford compounds 38 and 39: ESI-MS, m/z 431.1 and 445.2 (M+H)⁺.

### Example 40. Synthesis of (3-(4-(N-((1,2,3,5,6,7-hexahydro-s-indacen-4-yl)carbamoyl)sulfamoyl)-2-hydroxyphenyl)propyl)boronic acid

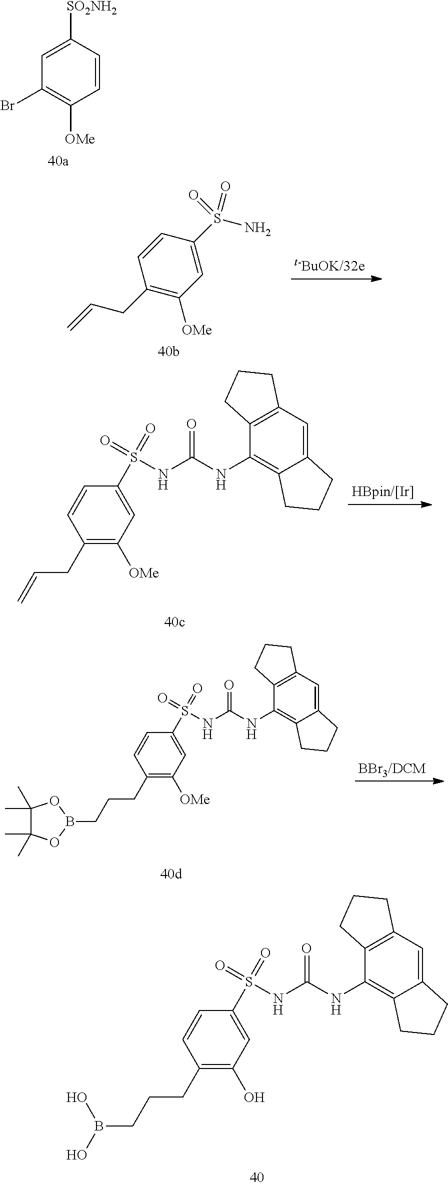 [SMILES: COc1ccc(S(N)(=O)=O)cc1Br ; C=CCc1ccc(S(N)(=O)=O)cc1OC ; C=CCc1ccc(S(=O)(=O)NC(=O)Nc2c3c(cc4c2CCC4)CCC3)cc1OC ; COc1cc(S(=O)(=O)NC(=O)Nc2c3c(cc4c2CCC4)CCC3)ccc1CCCB1OC(C)(C)C(C)(C)O1 ; O=C(Nc1c2c(cc3c1CCC3)CCC2)NS(=O)(=O)c1ccc(CCCB(O)O)c(O)c1]

### Step 1: Synthesis of 4-allyl-3-methoxybenzenesulfonamide (40b)

The title compound was prepared following the procedure described in Example 37, Step 1, using 4-bromo-3-methoxybenzenesulfonamide (40a) instead of 37a: ESI-MS, m/z 250.1 (M+H)⁺.

### Step 2: Synthesis of 4-allyl-N-((1,2,3,5,6,7-hexahydro-s-indacen-4-yl)carbamoyl)-3-methoxybenzenesulfonamide (40c)

The title compound was prepared following the procedure described in Example 32, Step 3, using 40b: ESI-MS, m/z 427.2 (M+H)⁺.

### Step 3: Synthesis of N-((1,2,3,5,6,7-hexahydro-s-indacen-4-yl)carbamoyl)-3-methoxy-4-(3-(4,4,5,5-tetramethyl-1,3,2-dioxaborolan-2-yl)propyl)benzenesulfonamide (40d)

The title compound was prepared following the procedure described in Example 32, Step 4, using 40c: ESI-MS, m/z 555.3 (M+H)⁺.

### Step 4: Synthesis of (3-(4-(N-((1,2,3,5,6,7-hexahydro-s-indacen-4-yl)carbamoyl)sulfamoyl)-2-hydroxyphenyl)propyl)boronic acid (40)

The title compound was prepared following the procedure described in Example 37, using 40d: ESI-MS, m/z 459.2 (M+H)⁺.

### Example 41. Synthesis of N-((1,2,3,5,6,7-hexahydro-s-indacen-4-yl)carbamoyl)-2-hydroxy-3,4-dihydro-2H-benzo[e][1,2]oxaborinine-7-sulfonamide

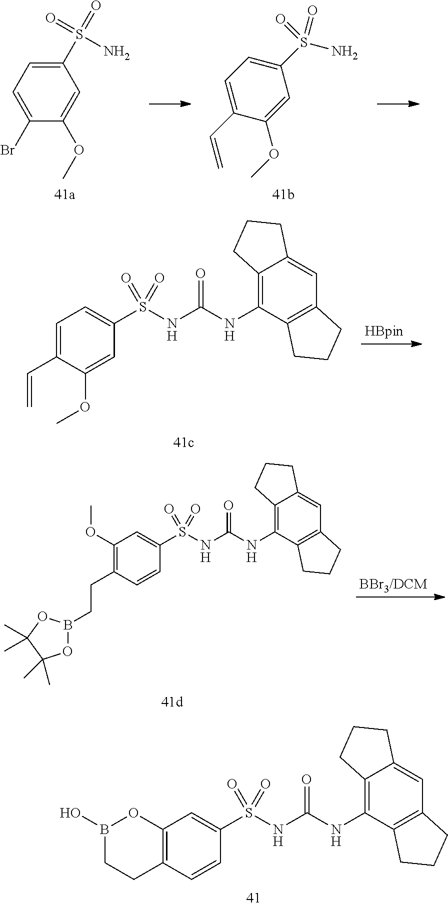

### Step 1: Synthesis of 3-methoxy-4-vinylbenzenesulfonamide (41b)

The title compound was prepared following the procedure described in Example 32, Step 2, using 41a instead of 32b as yellow solid: ESI-MS, m/z 214.1 (M+H)⁺.

### Step 2: Synthesis of N-((1,2,3,5,6,7-hexahydro-s-indacen-4-yl)carbamoyl)-3-methoxy-4-vinylbenzenesulfonamide (41c)

The title compound was prepared following the procedure described in Example 32, Step 3, using 41b as white solid: ESI-MS, m/z 413.2 (M+H)⁺.

### Step 3: Synthesis of N-((1,2,3,5,6,7-hexahydro-s-indacen-4-yl)carbamoyl)-3-methoxy-4-(2-(4,4,5,5-tetramethyl-1,3,2-dioxaborolan-2-yl)ethyl)benzenesulfonamide (41d)

The title compound was prepared following the procedure described in Example 32, Step 4, using 41c as white solid: ESI-MS, m/z 541.3 (M+H)⁺.

### Step 4: Synthesis of N-((1,2,3,5,6,7-hexahydro-s-indacen-4-yl)carbamoyl)-2-hydroxy-3,4-dihydro-2H-benzo[e][1,2]oxaborinine-7-sulfonamide

The title compound was prepared following the procedure described in Example 33 using 41d: ESI-MS, m/z 427.2 (M+H)⁺.

### Example 42. Synthesis of N-((1,2,3,5,6,7-hexahydro-s-indacen-4-yl)carbamoyl)-3-methoxy-4-(((3aS,4S,6S,7aR)-3a,5,5-trimethylhexahydro-4,6-methanobenzo[d][1,3,2]dioxaborol-2-yl)methyl)benzenesulfonamide

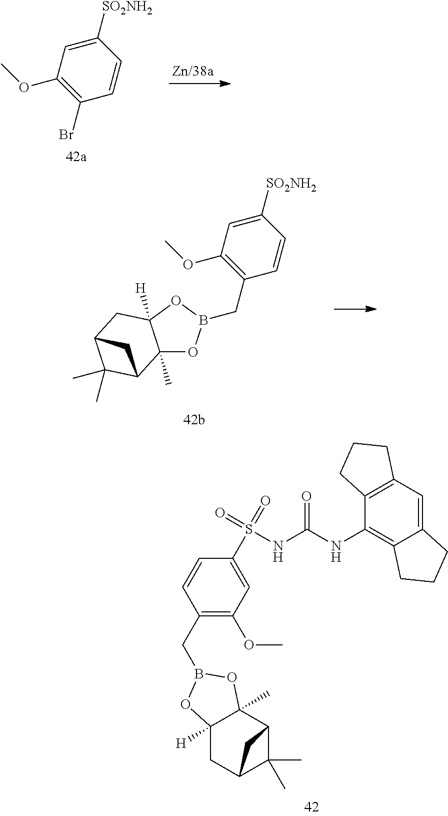 [SMILES: COc1cc(S(N)(=O)=O)ccc1Br ; COc1cc(S(N)(=O)=O)ccc1CB1O[C@@H]2C[C@@H]3C[C@@H](C3(C)C)[C@]2(C)O1 ; COc1cc(S(=O)(=O)NC(=O)Nc2c3c(cc4c2CCC4)CCC3)ccc1CB1O[C@@H]2C[C@@H]3C[C@@H](C3(C)C)[C@]2(C)O1]

### Step 1: Synthesis of 3-methoxy-4-(((3aS,4S,6S,7aR)-3a,5,5-trimethylhexahydro-4,6-methanobenzo[d][1,3,2]dioxaborol-2-yl)methyl)benzenesulfonamide (42b)

The title compound was prepared following the procedure described in Example 38, Step 1, using 42a and 38a: ESI-MS, m/z 402.1 (M+Na)⁺.

### Step 2: Synthesis of N-((1,2,3,5,6,7-hexahydro-s-indacen-4-yl)carbamoyl)-3-methoxy-4-(((3aS,4S,6S,7aR)-3a,5,5-trimethylhexahydro-4,6-methanobenzo[d][1,3,2]dioxaborol-2-yl)methyl)benzenesulfonamide

The title compound was prepared following the procedure described in Example 32, Step 3, using 42b: ESI-MS, m/z 579.3 (M+H)⁺.

### Example 43. Synthesis of (5-(N-((1,2,3,5,6,7-hexahydro-s-indacen-4-yl)carbamoyl)sulfamoyl)-2-methoxyphenyl)boronic acid

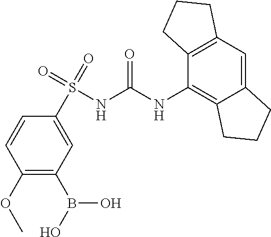 [SMILES: COc1ccc(S(=O)(=O)NC(=O)Nc2c3c(cc4c2CCC4)CCC3)cc1B(O)O]

The title compound was prepared following the procedure described in Example 1. ESI-MS, m/z 431.1 (M+H)⁺.

### Example 44. Synthesis of (4-(N-((1,2,3,5,6,7-hexahydro-s-indacen-4-yl)carbamoyl)sulfamoyl)-2-hydroxybenzyl)boronic acid

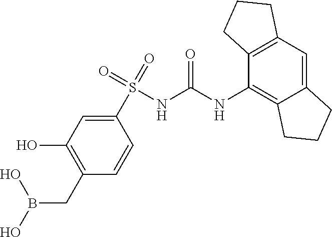 [SMILES: O=C(Nc1c2c(cc3c1CCC3)CCC2)NS(=O)(=O)c1ccc(CB(O)O)c(O)c1]

The title compound was prepared following the procedure described in Example 33 using 42 and BBr₃: ESI-MS, m/z 431.2 (M+H)⁺.

### Example 45. Synthesis of N-((1,2,3,5,6,7-hexahydro-s-indacen-4-yl)carbamoyl)-4-methoxy-3-(3-(4,4,5,5-tetramethyl-1,3,2-dioxaborolan-2-yl)propyl)benzenesulfonamide

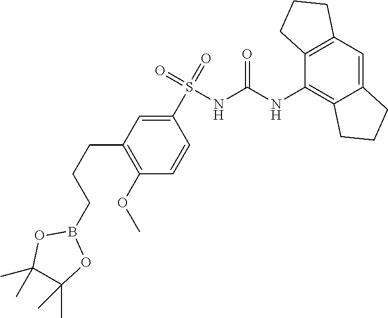 [SMILES: COc1ccc(S(=O)(=O)NC(=O)Nc2c3c(cc4c2CCC4)CCC3)cc1CCCB1OC(C)(C)C(C)(C)O1]

The title compound was prepared following the procedure described in Example 37, Step 3: ESI-MS, m/z 555.3 (M+H)⁺.

### Example 46. Synthesis of N-((1,2,3,5,6,7-hexahydro-s-indacen-4-yl)carbamoyl)-3-(3-(4,4,5,5-tetramethyl-1,3,2-dioxaborolan-2-yl)propyl)benzenesulfonamide

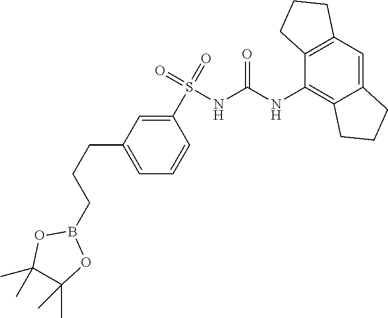 [SMILES: CC1(C)OB(CCCc2cccc(S(=O)(=O)NC(=O)Nc3c4c(cc5c3CCC5)CCC4)c2)OC1(C)C]

The title compound was prepared following the procedure described in Example 37, using 3-bromobenzenesulfonamide instead of 37a: ESI-MS, m/z 525.1 (M+H)⁺.

### Example 47. Synthesis of N-((1,2,3,5,6,7-hexahydro-s-indacen-4-yl)carbamoyl)-1-hydroxy-3,3-dimethyl-1,3-dihydrobenzo[c][1,2]oxaborole-6-sulfonamide

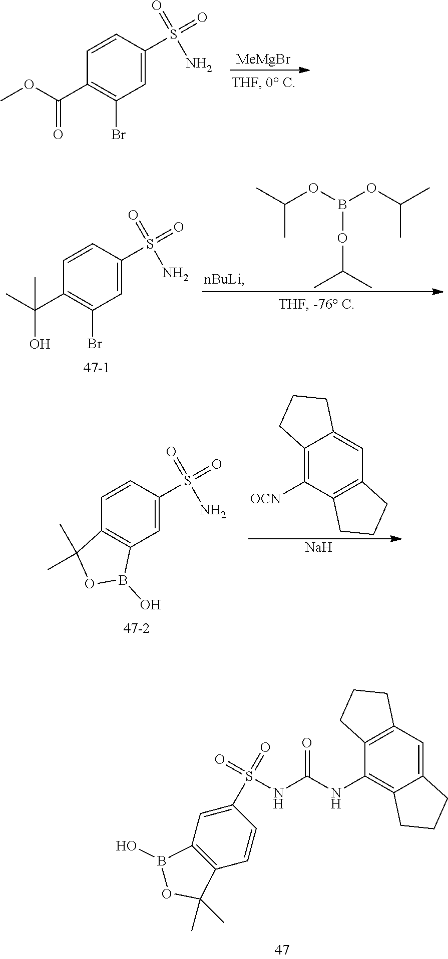

### Step 1. Synthesis of 3-bromo-4-(2-hydroxypropan-2-yl)benzenesulfonamide (47-1)

To compound 35-1 (2.2 g, 7.5 mmol) in THF (50 mL) was added MeMgBr (45 mmol) at −10° C. The resulting mixture was allowed to warm up to RT and was stirred at RT for 1 h. The reaction was quenched by addition of sat. NH₄Cl. The aqueous phase was extracted with EtOAC. The organic phase was washed with brine, dried over Na₂SO₄, and concentrated. The residue was purified by flash chromatography to afford the titled product as white solid (1.0 g).

### Step 2. Synthesis of 1-hydroxy-3,3-dimethyl-1,3-dihydrobenzo[c][1,2]oxaborole-6-sulfonamide (47-2)

To compound 47-1 (1.0 g, 3.4 mmol) in THF (20 mL) at −78° C. was added nBuLi (20 mmol) slowly and the reaction mixture was stirred at same temperature for 1 h. Triisopropyl borate (3.3 mL, 13 mmol) was added at −76° C. and the reaction mixture was stirred and allowed to warm up to RT. The reaction mixture was cooled at 0° C. and 1 N HCl (10 mL) was added to quench the reaction. The aqueous phase was extracted with EtOAC. The organic phase was washed with brine, dried over Na₂SO₄, and concentrated. The residue was purified by flash chromatography to afford the titled product (0.15 g).

### Step 3. Synthesis of N-((1,2,3,5,6,7-hexahydro-s-indacen-4-yl)carbamoyl)-1-hydroxy-3,3-dimethyl-1,3-dihydrobenzo[c][1,2]oxaborole-6-sulfonamide (47)

The title compound was prepared following the procedure described in Example 1, Step 2. ESI-MS, m/z 441.2 (M+H)⁺.

### Example 48. Synthesis of N-((1,2,3,5,6,7-hexahydro-s-indacen-4-yl)carbamoyl)-3-hydroxy-4-(3-((3aS,4S,6S,7aR)-3a,5,5-trimethylhexahydro-4,6-methanobenzo[d][1,3,2]dioxaborol-2-yl)propyl)benzenesulfonamide

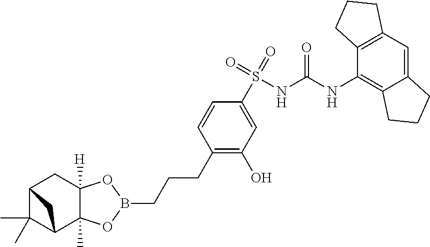 [SMILES: CC1(C)[C@@H]2C[C@H]3OB(CCCc4ccc(S(=O)(=O)NC(=O)Nc5c6c(cc7c5CCC7)CCC6)cc4O)O[C@@]3(C)[C@H]1C2]

A mixture of 32 (10 mg) and (+)-pinanediol (5 mg) in THF was stirred at RT for 20 h. The solvent was removed and purified by reverse phase HPLC to afford the title compound as white solid. ESI-MS, m/z 593.3 (M+H)⁺.

### Example 49. Synthesis of N-((1,2,3,5,6,7-hexahydro-s-indacen-4-yl)carbamoyl)-4-hydroxy-3-(3-((3aS,4S,6S,7aR)-3a,5,5-trimethylhexahydro-4,6-methanobenzo[d][1,3,2]dioxaborol-2-yl)propyl)benzenesulfonamide

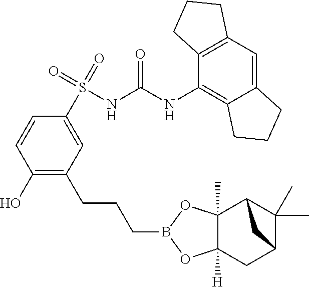 [SMILES: CC1(C)[C@@H]2C[C@H]3OB(CCCc4cc(S(=O)(=O)NC(=O)Nc5c6c(cc7c5CCC7)CCC6)ccc4O)O[C@@]3(C)[C@H]1C2]

Compound 49 was prepared following the procedure described in Example 48 using 37 instead of 32: ESI-MS, m/z 593.3 (M+H)⁺.

### Example 50. Synthesis of N-((1,2,3,5,6,7-hexahydro-s-indacen-4-yl)carbamoyl)-4-methoxy-3-(3-((3aS,4S,6S,7aR)-3a,5,5-trimethylhexahydro-4,6-methanobenzo[d][1,3,2]dioxaborol-2-yl)propyl)benzenesulfonamide

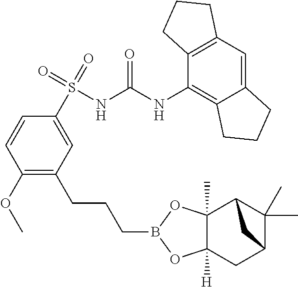 [SMILES: COc1ccc(S(=O)(=O)NC(=O)Nc2c3c(cc4c2CCC4)CCC3)cc1CCCB1O[C@@H]2C[C@@H]3C[C@@H](C3(C)C)[C@]2(C)O1]

Compound 50 was prepared following the same procedure described in Example 48 using 37d instead of 32: ESI-MS, m/z 607.3 (M+H)⁺.

### Example 51. Synthesis of 1-hydroxy-N-((5-(2-methoxypyridin-4-yl)-2,3-dihydro-1H-inden-4-yl)carbamoyl)-1,3-dihydrobenzo[c][1,2]oxaborole-5-sulfonamide

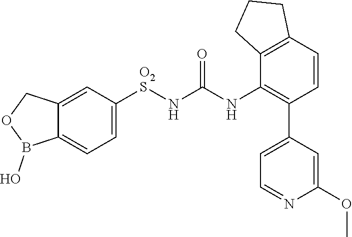 [SMILES: COc1cc(-c2ccc3c(c2NC(=O)NS(=O)(=O)c2ccc4c(c2)COB4O)CCC3)ccn1]

The title compound was prepared following the procedures described in Example 29 using 4-(4-isocyanato-2,3-dihydro-1H-inden-5-yl)-2-methoxypyridine in Step 6. The final product was purified by prep-HPLC (ACN:H₂O:NH₃·H₂O (0.05%), 10% to 40%) to afford as white solid. ESI-MS, m/z 479.8 (M+H)⁺.

### Example 52. Synthesis of N-((4-cyano-3-fluoro-2,6-diisopropylphenyl)carbamoyl)-1,3-dihydroxy-1,3-dihydrobenzo[c][1,2]oxaborole-5-sulfonamide

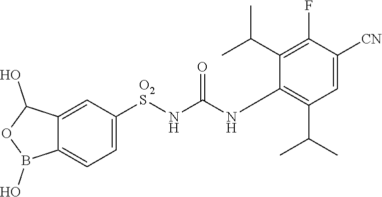 [SMILES: CC(C)c1cc(C#N)c(F)c(C(C)C)c1NC(=O)NS(=O)(=O)c1ccc2c(c1)C(O)OB2O]

The title compound was prepared following the procedures described in Example 29 using intermediate 54e (Example 54, Step 4) and 2-fluoro-4-isocyanato-3,5-diisopropylbenzonitrile in Step 6. The final product was purified by prep-HPLC (ACN:H₂O:NH₃—H₂O (0.05%), 10% to 40%) to afford as white solid. ESI-MS, m/z 475.9 (M+H)⁺.

### Example 53. Synthesis of 1,3-dihydroxy-N-((5-(2-methoxypyridin-4-yl)-2,3-dihydro-1H-inden-4-yl)carbamoyl)-1,3-dihydrobenzo[c][1,2]oxaborole-5-sulfonamide

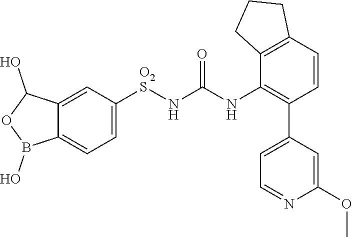 [SMILES: COc1cc(-c2ccc3c(c2NC(=O)NS(=O)(=O)c2ccc4c(c2)C(O)OB4O)CCC3)ccn1]

The title compound was prepared following the procedures described in Example 29, using intermediate 54e (Example 54, Step 4) and 4-(4-isocyanato-2,3-dihydro-1H-inden-5-yl)-2-methoxypyridine (PCT Int. Appl., 2020035464, 20 Feb. 2020) in Step 6. The final product was purified by prep-HPLC (ACN:H₂O:NH₃·H₂O (0.05%), 10% to 40%) to afford as white solid. ESI-MS, m/z 495.9 (M+H)⁺.

### Example 54. Synthesis of N-((1,2,3,5,6,7-hexahydro-s-indacen-4-yl)carbamoyl)-1,3-dihydroxy-1,3-dihydrobenzo[c][1,2]oxaborole-5-sulfonamide

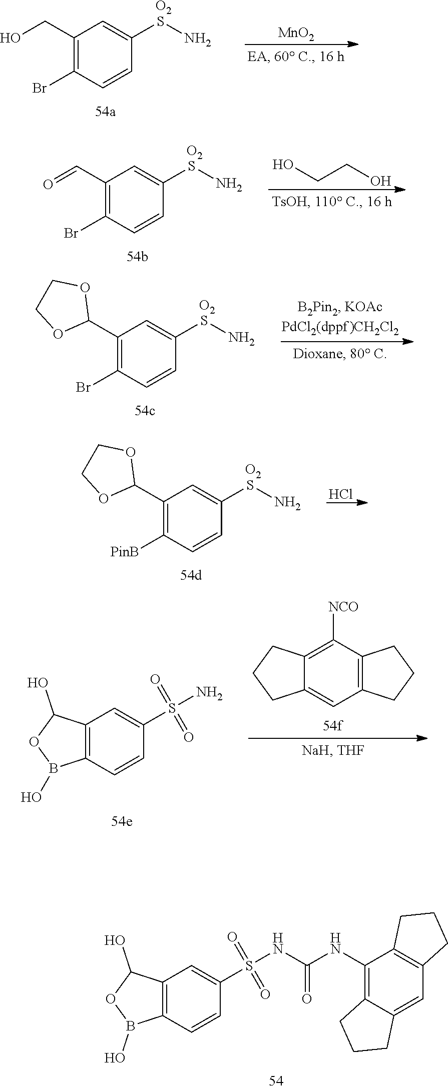

### Step 1: Synthesis of 4-bromo-3-formylbenzenesulfonamide (54b)

To a solution of 54a (80 g, 302 mmol) in EA (800 mL) was added MnO₂ (263 g, 3020 mmol) at rt. And then the resulting mixture was stirred at 60° C. for 16 h, filtered and the organic layer was concentrated to afford the product as white solid (60 g). Yield: 76%. ESI-MS, m/z 264.1 (M+H)⁺.

### Step 2: Synthesis of 4-bromo-3-(1,3-dioxolan-2-yl)benzenesulfonamide (54c)

To a solution of 54b (20 g, 76 mmol) in toluene (300 mL) were added ethylene glycol (21 g, 341 mmol) and TsOH (1.3 g, 7.6 mmol) at rt. And then the resulting mixture was stirred at 110° C. for 16 h. The mixture was concentrated and purified by FCC (PE:EA=50%) to afford the product as white solid (10 g). Yield: 43%. ESI-MS, m/z 308.0 (M+H)⁺.

### Step 3: Synthesis of 3-(1,3-dioxolan-2-yl)-4-(4,4,5,5-tetramethyl-1,3,2-dioxaborolan-2-yl)benzenesulfonamide (54d)

The title compound was prepared following the procedures described in Example 29, Step 5, using 54c. The final product was purified by flash chromatography on silica gel (EtOAc/Hexanes 0˜50%) to afford 54d as yellow solid (3.9 g). Yield: 68% ESI-MS, m/z 356.1 (M+H)⁺.

### Step 4: Synthesis of 1,3-dihydroxy-1,3-dihydrobenzo[c][1,2]oxaborole-5-sulfonamide (54e)

To a solution of 54d (2 g, 5.6 mmol) in THF (10 mL) was added 1 N HCl (15 mL) at 0° C. And then the resulting mixture was stirred at RT for 4 h. The mixture was extracted with EA (3×15 mL), the water layer was purified by prep-HPLC (ACN:H₂O:NH₃·H₂O (0.05%)=10% to 40%) to afford the product as white solid (600 mg). Yield: 46%. ESI-MS, m/z 230.0 (M+H)⁺.

### Step 5: Synthesis of N-((1,2,3,5,6,7-hexahydro-s-indacen-4-yl)carbamoyl)-1,3-dihydroxy-1,3-dihydrobenzo[c][1,2]oxaborole-5-sulfonamide

The title compound was prepared following the procedures described in Example 29, Steps 1-7, using 54e. The final product was purified by prep-HPLC (ACN:H₂O:NH₃·H₂O (0.05%)=10% to 40%) to afford the title compound as white solid (24.7 mg). Yield: 6% ESI-MS, m/z 428.9 (M+H)⁺.

### Example 55. Synthesis of 1-hydroxy-N-((5-(2-methoxypyridin-4-yl)-2,3-dihydro-1H-inden-4-yl)carbamoyl)-3-methyl-1,3-dihydrobenzo[c][1,2]oxaborole-5-sulfonamide

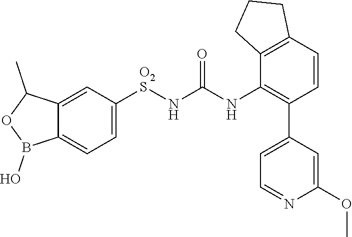 [SMILES: COc1cc(-c2ccc3c(c2NC(=O)NS(=O)(=O)c2ccc4c(c2)C(C)OB4O)CCC3)ccn1]

The title compound was prepared following the procedures described in Example 56, using 56d and 4-(4-isocyanato-2,3-dihydro-1H-inden-5-yl)-2-methoxypyridine in Step 4. The final product was purified by prep-HPLC (ACN:H₂O:NH₃·H₂O (0.05%), 10% to 40%) to afford as white solid. ESI-MS, m/z 493.8 (M+H)⁺.

### Example 56. Synthesis of N-((4-cyano-3-fluoro-2,6-diisopropylphenyl)carbamoyl)-1-hydroxy-3-methyl-1,3-dihydrobenzo[c][1,2]oxaborole-5-sulfonamide

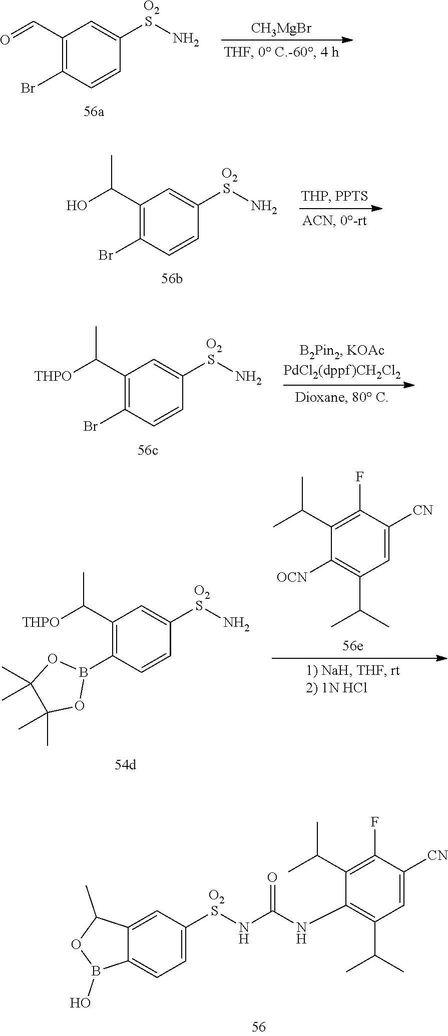

### Step 1: Synthesis of 4-bromo-3-(1-hydroxyethyl)benzenesulfonamide (56b)

To a solution of 56a (17 g, 65 mmol) in THF (200 mL) was added CH₃MgBr (120 mL, 360 mmol) at 0° C. under nitrogen. The resulting mixture was then stirred at 60° C. for 16 h. The mixture was quenched by aq. NH₄Cl (150 mL), extracted with EA (3×150 mL). The organic layers were dried over Na₂SO₄, filtered. The mixture was concentrated and purified by FCC (PE:EA=40%) to afford the product as white solid (14 g). Yield: 77%. ESI-MS, m/z 261.7 (M-OH)⁺.

### Step 2: Synthesis of 4-bromo-3-(1-((tetrahydro-2H-pyran-2-yl)oxy)ethyl)benzenesulfonamide (56c)

The title compound was prepared following the procedures described in Example 29, Step 4, using 56b. The mixture was concentrated and purified by FCC (PE:EA=40%) to afford the product as white solid (5.5 g). Yield: 30%. ESI-MS, m/z 386 (M+Na)⁺.

### Step 3: Synthesis of 3-(1-((tetrahydro-2H-pyran-2-yl)oxy)ethyl)-4-(4,4,5,5-tetramethyl-1,3,2-dioxaborolan-2-yl)benzenesulfonamide (56d)

The title compound was prepared following the procedures described in Example 29, Step 5, using 56c. The mixture was concentrated and purified by FCC (PE:EA=40%) to afford the product as white solid (2.5 g). Yield: 33%. ESI-MS, m/z 424 (M+Na)⁺.

### Step 4. Synthesis of N-((4-cyano-3-fluoro-2,6-diisopropylphenyl)carbamoyl)-1-hydroxy-3-methyl-1,3-dihydrobenzo[c][1,2]oxaborole-5-sulfonamide

The title compound was prepared following the procedures described in v, using 56d and 2-fluoro-4-isocyanato-3,5-diisopropylbenzonitrile (56e). The final product was purified by prep-HPLC (ACN:H₂O:NH₃·H₂O (0.05%), 10% to 40%) to afford as white solid. ESI-MS, m/z 473.9 (M+H)⁺.

### Example 57. Synthesis of N-((1,2,3,5,6,7-hexahydro-s-indacen-4-yl)carbamoyl)-1-hydroxy-3,3-dimethyl-1,3-dihydrobenzo[c][1,2]oxaborole-5-sulfonamide

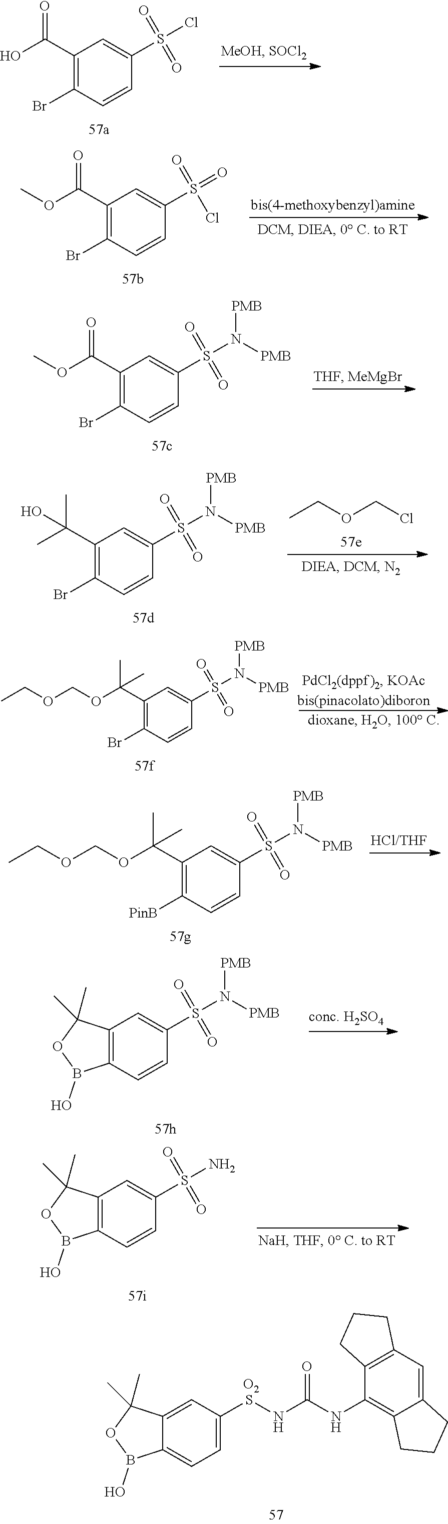

### Step 1: Synthesis of methyl 2-bromo-5-(chlorosulfonyl)benzoate (57b)

To a solution of 57a (6 g, 30 mmol) in MeOH (60 mL), SOCl₂ (10.7 g, 90 mmol) was slowly added at 0° C. And then the resulting mixture was stirred at 80° C. for 3 h. Concentrated and the residue was concentrated and purified by FCC (PE:EA=5%) to afford the product as colorless oil (6.5 g). Yield: 100%. ESI-MS, m/z 214.8 (M+H)⁺.

### Step 2: Synthesis of methyl 5-(N,N-bis(4-methoxybenzyl)sulfamoyl)-2-bromobenzoate (57c)m

To a solution of 57b (6.6 g, 21 mmol) in DCM (70 mL), DIEA (3.3 g, 25 mmol), Bn₂NH (4.6 g, 23 mmol) were added at 0° C. And then the resulting mixture was stirred at RT for 2 h, then concentrated and the residue was purified by FCC (1% MeOH/DCM) to afford the product as white solid (6.9 g). Yield: 69%. ESI-MS, m/z 474.0 (M+H)⁺.

### Step 3: Synthesis of 4-bromo-3-(2-hydroxypropan-2-yl)-N,N-bis(4-methoxybenzyl) benzenesulfonamide (57d)

To a solution of 57c (10.7 g, 20 mmol) in THF (100 mL), MeMgBr (20 mL, 60 mmol) was slowly added at 0° C. under nitrogen. And then the resulting mixture was stirred at RT for 7 h. Quenched by aq. NH₄Cl (50 mL), extracted with EA (3×50 mL), dried over Na₂SO₄, filtered. Concentrated and the residue was concentrated and purified by FCC (15% EA/PE) to afford the product as colorless oil (6.2 g). Yield: 58%. ESI-MS, m/z 555.6 (M+Na)⁺.

### Step 4: Synthesis of 4-bromo-3-(2-(ethoxymethoxy)propan-2-yl)-N,N-bis(4-methoxybenzyl) benzenesulfonamide (57f)

To a solution of 57d (7.1 g, 15 mmol) in DCM (70 mL) were added DIEA (5.8 g, 45 mmol), 57e (2.8 g, 30 mmol) at RT under nitrogen. And then the resulting mixture was stirred at 40° C. for 24 h. Concentrated and the residue was concentrated and purified by FCC (11% EA/PE) to afford the product as colorless oil (7.9 g). Yield: 98%. ESI-MS, m/z 553.6 (M+Na)⁺.

### Step 5: Synthesis of 3-(2-(ethoxymethoxy)propan-2-yl)-N,N-bis(4-methoxybenzyl)-4-(4,4,5,5-tetramethyl-1,3,2-dioxaborolan-2-yl)benzenesulfonamide (57g)

The title compound was prepared following the procedures described in Example 29. The crude product was concentrated and purified by FCC (30% EA/PE) to afford the product as colorless oil (7 g). Yield: 70%. ESI-MS, m/z 601.8 (M+Na)⁺.

### Step 6: Synthesis of 1-hydroxy-N,N-bis(4-methoxybenzyl)-3,3-dimethyl-1,3-dihydrobenzo[c][1,2]oxaborole-5-sulfonamide (57h)

To a solution of 57g (23 g, 36 mmol) in THF (90 mL) was added 6N HCl (90 mL) at rt. And then the resulting mixture was stirred at RT for 24 h. Extracted with EA (3×50 mL), dried over Na₂SO₄, filtered. Concentrated and the residue was purified by FCC (3% MeOH/DCM) to afford the product as colorless oil (16 g). Yield: 91%. ESI-MS, m/z 503.4 (M+Na)⁺.

### Step 7: Synthesis 1-hydroxy-3,3-dimethyl-1,3-dihydrobenzo[c][1,2]oxaborole-5-sulfonamide (57i)

A solution of 57h (16 g, 16.6 mmol) in H₂SO₄ (25 mL) was stirred at RT for 1 h. Poured into ice water (100 mL). Extracted with EA (3×100 mL), dried over Na₂SO₄, filtered. Concentrated and the residue was concentrated and purified by FCC (5% MeOH/DCM) to afford the product as colorless oil (5.7 g). Yield: 72%. ESI-MS, m/z 241.9 (M+H)⁺.

### Step 8: Synthesis of 2-(1,2,3,5,6,7-hexahydro-s-indacen-4-yl)-N-((1-hydroxy-3,3-dimethyl-1,3-dihydrobenzo[c][1,2]oxaborol-5-yl)sulfonyl)acetamide

The title compound was prepared following the procedures described in Example 29, Steps 6-7. The final product was purified by prep-HPLC (ACN:H₂O:NH₃·H₂O (0.05%), 10% to 40%) to afford as white solid. ESI-MS, m/z 440.7 (M+H)⁺.

### Example 58. Synthesis of N-((1,2,3,5,6,7-hexahydro-s-indacen-4-yl)carbamoyl)-4-(1-(4,4,5,5-tetramethyl-1,3,2-dioxaborolan-2-yl)propan-2-yl)furan-2-sulfonamide

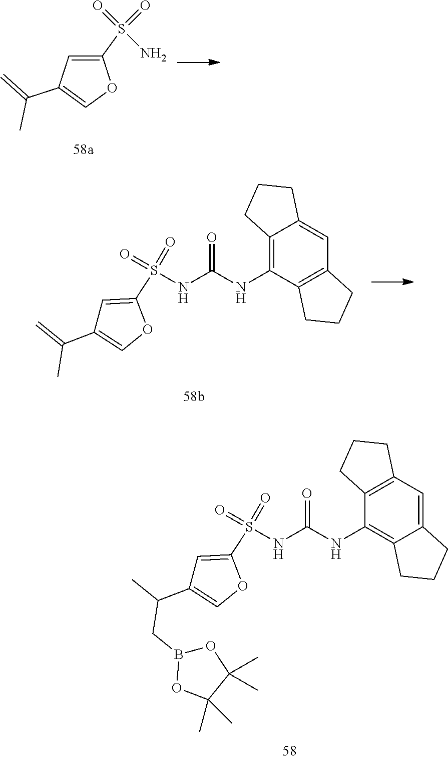 [SMILES: C=C(C)c1coc(S(N)(=O)=O)c1 ; C=C(C)c1coc(S(=O)(=O)NC(=O)Nc2c3c(cc4c2CCC4)CCC3)c1 ; CC(CB1OC(C)(C)C(C)(C)O1)c1coc(S(=O)(=O)NC(=O)Nc2c3c(cc4c2CCC4)CCC3)c1]

### Step 1: Synthesis of N-((1,2,3,5,6,7-hexahydro-s-indacen-4-yl)carbamoyl)-4-(prop-1-en-2-yl)furan-2-sulfonamide

Compound 58b was prepared following the literature procedure (O'Nell, Luke et al., PCT Int. Appl. WO2016/131098).

### Step 2: Synthesis of N-((1,2,3,5,6,7-hexahydro-s-indacen-4-yl)carbamoyl)-4-(1-(4,4,5,5-tetramethyl-1,3,2-dioxaborolan-2-yl)propan-2-yl)furan-2-sulfonamide

Compound 58 was prepared following the procedure described in Example 32, Step 4, using 59b instead of 32d: ESI-MS, m/z 515.3 (M+H)⁺.

### Example 59. Synthesis of N-((1,2,3,5,6,7-hexahydro-s-indacen-4-yl)carbamoyl)-1-(3-(4,4,5,5-tetramethyl-1,3,2-dioxaborolan-2-yl propyl)-1H-pyrazole-3-sulfonamide

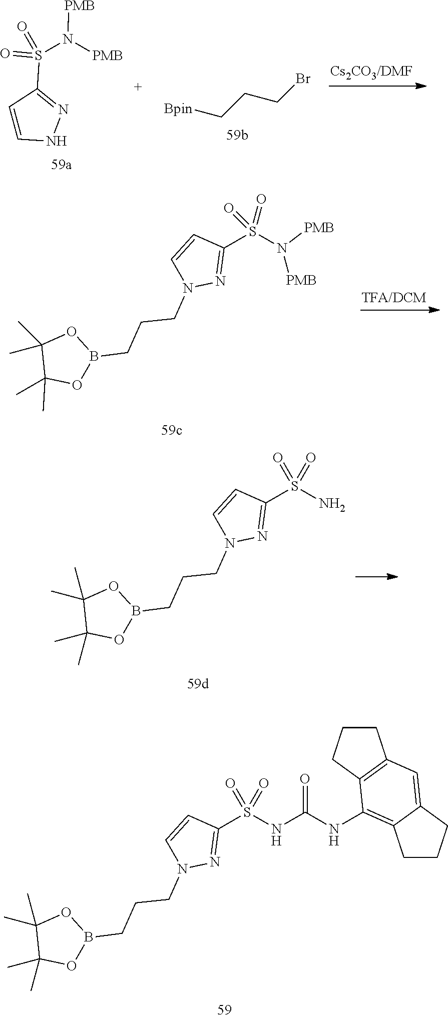

### Step 1: Synthesis of N,N-bis(4-methoxybenzyl)-1-(3-(4,4,5,5-tetramethyl-1,3,2-dioxaborolan-2-yl)propyl)-1H-pyrazole-3-sulfonamide

59b (0.65 g, 2.6 mmol) was added to a mixture of 59a (1 g, 2.6 mmol, Cooper, M., et al., PCT Int. Appl. WO 2020/104657) and Cs₂CO₃ (1.2 g, 3.7 mmol) in DMF (5 mL) at 0° C. The resulting mixture was warmed to RT for 48 h. The reaction mixture was quenched with water and extracted with EtOAc. The organic layer was washed with water, brine, dried over Na₂SO₄, concentrated, and purified by flash chromatography column (EtOAc/hexanes 0˜ 80%) to give 59c as colorless oil (0.25 g): ESI-MS, m/z 556.2 (M+H)⁺.

### Step 2: Synthesis of 1-(3-(4,4,5,5-tetramethyl-1,3,2-dioxaborolan-2-yl)propyl)-1H-pyrazole-3-sulfonamide

TFA (3 mL) was added to a solution of 59c (0.25 g) in DCM (1.5 mL) at 0° C. under argon. After 30 min., warmed to 30° C. for 2 h. The reaction mixture was concentrated under vacuum and purified via reverse phase chromatography (CH₃CN (0.1% TFA)/water (0.1% TFA) 10˜100%) to afford 59d as white solid: ESI-MS, m/z 316.2 (M+H)⁺.

### Step 3: Synthesis of N-((1,2,3,5,6,7-hexahydro-s-indacen-4-yl)carbamoyl)-1-(3-(4,4,5,5-tetramethyl-1,3,2-dioxaborolan-2-yl)propyl)-1H-pyrazole-3-sulfonamide

Compound 59 was prepared following the procedure described in Example 32, Step 3, using 59d instead of 32c: ESI-MS, m/z 515.2 (M+H)⁺.

### Example 60. Synthesis of N-((1,2,3,5,6,7-hexahydro-s-indacen-4-yl)carbamoyl)-1-(4-((3aS,4S,6S,7aR)-3a,5,5-trimethylhexahydro-4,6-methanobenzo[d][1,3,2]dioxaborol-2-yl)butan-2-yl)-1H-pyrazole-3-sulfonamide

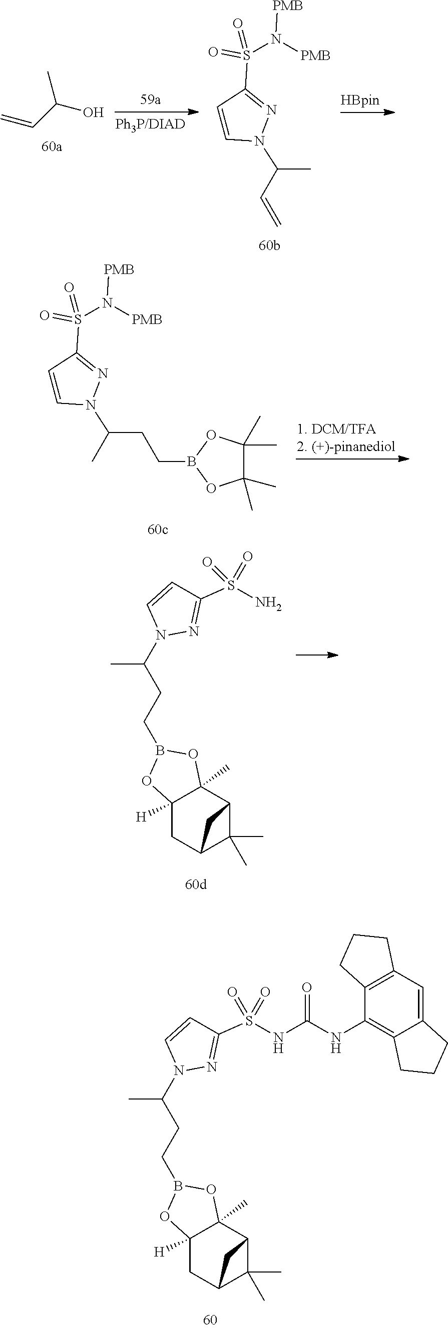

### Step 1: Synthesis of 1-(but-3-en-2-yl)-N,N-bis(4-methoxybenzyl)-1H-pyrazole-3-sulfonamide (60b)

DIAD (0.86 g, 4.3 mmol) was added dropwise to a mixture of 60a (1.5 g, 3.9 mmol), Ph₃P (1.0 g, 3.8 mmol) and 59a (0.32 g) in THF (10 mL) below 10° C. The reaction mixture was warmed to RT overnight, quenched with water and extracted with EtOAc. The organic layer was washed with brine, concentrated and purified by flash chromatography column (EtOAc/Hexane 0˜80%) to give 60b (the major product 0.9 g) as colorless oil: ESI-MS, m/z 442.2 (M+H)⁺.

### Step 2: Synthesis of N,N-bis(4-methoxybenzyl)-1-(4-(4,4,5,5-tetramethyl-1,3,2-dioxaborolan-2-yl)butan-2-yl)-1H-pyrazole-3-sulfonamide (60c)

Compound 60c was prepared following the procedure described in Example 32, Step 4, using 60b instead of 32d: ESI-MS, m/z 570.3 (M+H)⁺.

### Step 3: Synthesis of 1-(4-((3aS,4S,6S,7aR)-3a,5,5-trimethylhexahydro-4,6-methanobenzo[d][1,3,2]dioxaborol-2-yl)butan-2-yl)-1H-pyrazole-3-sulfonamide

TFA (2 mL) was added to a solution of 60c (75 mg, 0.13 mmol) in DCM (0.4 mL) at 0° C. After 1 h at rt, the reaction mixture was warmed to 40° C. for 1 h and 70° C. for 2 h. The reaction mixture was concentrated and dried using lyophilization. The brown solid and (+)-pinanediol (50 mg, 0.29 mmol) in THE (2 mL) was stirred at RT for 2 days. The solvent was removed, and the residue was purified via reverse phase chromatography (CH₃CN (0.1% TFA)/water (0.1% TFA) 10˜100%) to afford 60d as white solid (30 mg): ESI-MS, m/z 382.1 (M+H)⁺.

### Step 4: Synthesis of N-((1,2,3,5,6,7-hexahydro-s-indacen-4-yl)carbamoyl)-1-(4-((3aS,4S,6S,7aR)-3a,5,5-trimethylhexahydro-4,6-methanobenzo[d][1,3,2]dioxaborol-2-yl)butan-2-yl)-1H-pyrazole-3-sulfonamide

The title compound was prepared following the procedure described in Example 32, Step 3, using 60d and 4-isocyanato-1,2,3,5,6,7-hexahydro-s-indacene: ESI-MS, m/z 581.2 (M+H)⁺.

### Example 61. N-((1,2,3,5,6,7-hexahydro-s-indacen-4-yl)carbamoyl)-1-(3-methyl-4-(4,4,5,5-tetramethyl-1,3,2-dioxaborolan-2-yl)butyl)-1H-pyrazole-3-sulfonamide

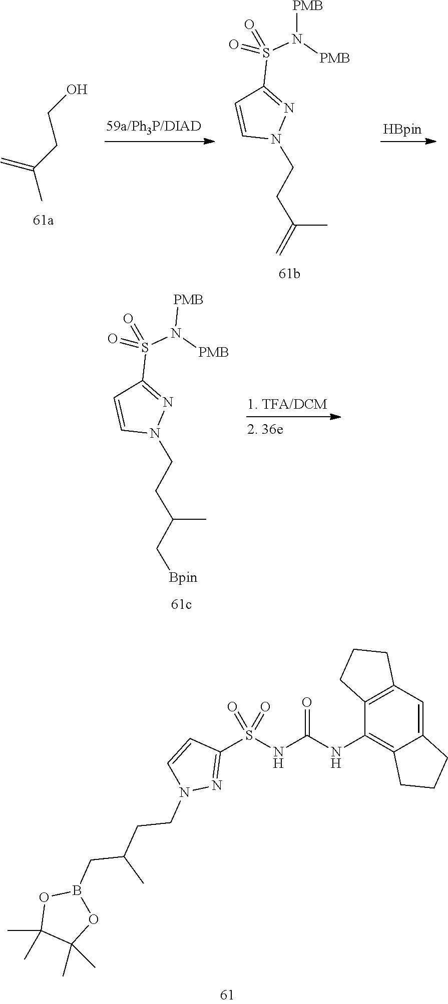

### Step 1: Synthesis of N,N-bis(4-methoxybenzyl)-1-(3-methylbut-3-en-1-yl)-1H-pyrazole-3-sulfonamide (61b)

The title compound was prepared following the procedure described in Example 60, Step 1, using 61a instead of 60a as colorless oil: ESI-MS, m/z 456.2 (M+H)⁺.

### Step 2: Synthesis of N,N-bis(4-methoxybenzyl)-1-(3-methyl-4-(4,4,5,5-tetramethyl-1,3,2-dioxaborolan-2-yl)butyl)-1H-pyrazole-3-sulfonamide (61c)

The title compound was prepared following the procedure described in Example 60, Step 2, using 61b instead of 60b: ESI-MS, m/z 584.2 (M+H)⁺.

### Step 3: Synthesis of N-((1,2,3,5,6,7-hexahydro-s-indacen-4-yl)carbamoyl)-1-(3-methyl-4-(4,4,5,5-tetramethyl-1,3,2-dioxaborolan-2-yl)butyl)-1H-pyrazole-3-sulfonamide

The title compound was prepared following the procedure described in Example 60, Steps 3 and 4, using 61c instead of 60c: ESI-MS, m/z 543.2 (M+H)⁺.

### Example 62. N-((1,2,3,5,6,7-hexahydro-s-indacen-4-yl)carbamoyl)-1-(3-methyl-3-(4,4,5,5-tetramethyl-1,3,2-dioxaborolan-2-yl)butyl)-1H-pyrazole-3-sulfonamide

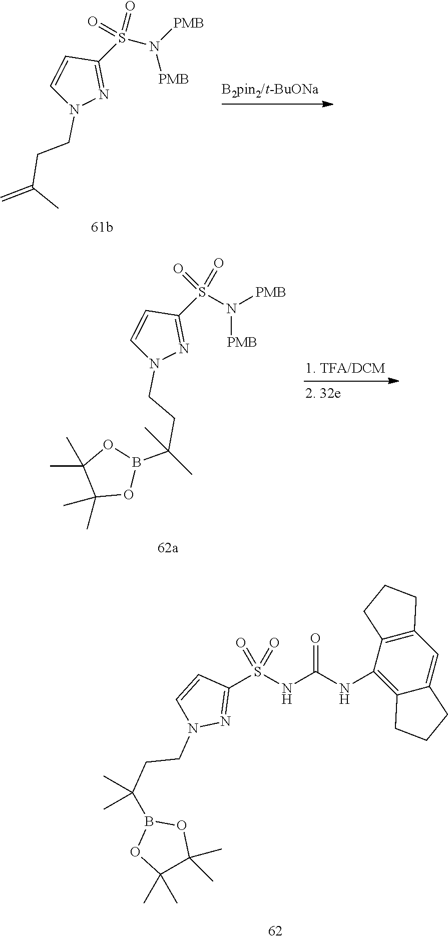

### Step 1: Synthesis of N,N-bis(4-methoxybenzyl)-1-(3-methyl-3-(4,4,5,5-tetramethyl-1,3,2-dioxaborolan-2-yl)butyl)-1H-pyrazole-3-sulfonamide

A mixture of t-BuONa (29 mg, 0.3 mmol) and i-PrCuCl (15 mg, 0.03 mmol) in DCM was stirred at RT for 10 min. under argon. B₂Pin₂ (150 mg, 0.6 mmol) was added, and the mixture was stirred for an additional 30 minutes at rt, followed by the addition of a solution of 61b (50 mg, 0.1 mmol) and MeOH (9 mg, 0.3 mmol) in DCM (0.5 mL). After 20 h at rt, the reaction was diluted with DCM (4 mL) and quenched with sat. aq. NH₄Cl The DCM layer was filtered over Na₂SO₄, concentrated, and purified by silica flash chromatography (EtOAc/hexane 0˜70%) to give 62a as colorless oil (10 mg): ESI-MS, m/z 584.2 (M+H)⁺.

### Step 2: Synthesis of N-((1,2,3,5,6,7-hexahydro-s-indacen-4-yl)carbamoyl)-1-(3-methyl-3-(4,4,5,5-tetramethyl-1,3,2-dioxaborolan-2-yl)butyl)-1H-pyrazole-3-sulfonamide

The title compound was prepared following the procedure described in Example 60, Steps 3 and 4, using 62a instead of 60c: ESI-MS, m/z 543.2 (M+H)⁺.

### Example 63. Synthesis of (3-(3-(N-((1,2,3,5,6,7-hexahydro-s-indacen-4-yl)carbamoyl)sulfamoyl)-1H-pyrazol-1-yl)propyl)boronic acid

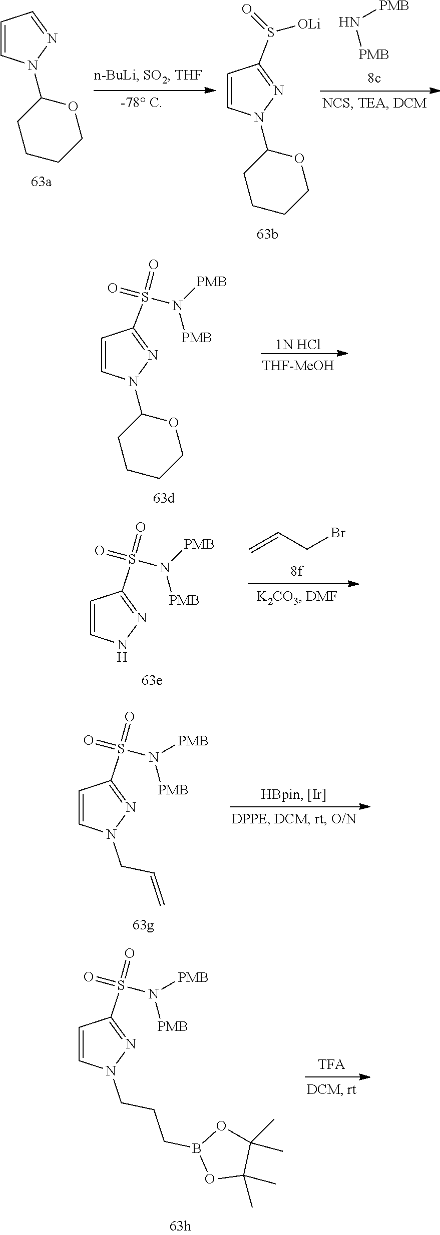

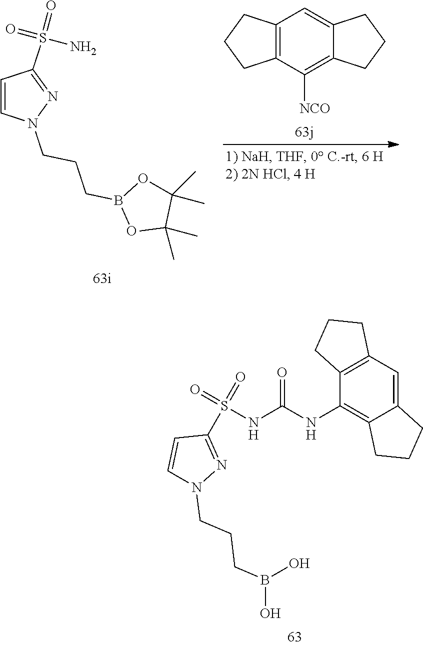

### Step 1: Synthesis of lithium 1-(tetrahydro-2H-pyran-2-yl)-1H-pyrazole-3-sulfinate (63b)

To a solution of 63a (108 g, 710 mmol) in THF (800 mL), n-BuLi (310 mL, 746 mmol) was slowly added at −65° C. under nitrogen. And then the resulting mixture was stirred at −78° C. for 1.5 h. Then sulfur dioxide was bubbled through for 10 min. And allowed to warm to rt. Filtered and triturated with TBME (3×150 mL) to afford the product as white solid (166 g crude). ESI-MS, m/z 215.1 (M-Li)⁻.

### Step 2: Synthesis of N,N-bis(4-methoxybenzyl)-1-(tetrahydro-2H-pyran-2-yl)-1H-pyrazole-3-sulfonamide (63d)

To a solution of 63b (51 g, 230 mmol) in DCM (800 mL), NCS (31 g, 230 mmol) was added at 0° C. under nitrogen. The resulting mixture was stirred at 0° C. for 4 h, then quenched by addition of water (200 mL). The mixture was extracted with DCM (2×200 mL), and the organic layer was concentrated to 50-80 mL. The solution was added to a mixture of 63c (59 g, 230 mmol) and E₃tN (77 g, 758 mmol) in DCM (200 mL) cooled in an ice bath. After stirred for 1 h, the reaction was warmed to rt, extracted with DCM (2×200 mL), and the organic layers were dried over Na₂SO₄, filtered, and concentrated to afford the product as white oil (100 g). Yield: 93%. ESI-MS, m/z 470.0 (M+H)⁺.

### Step 3: Synthesis of N,N-bis(4-methoxybenzyl)-1H-pyrazole-3-sulfonamide (63e)

To a solution of 63d (5 g, 10.6 mmol) in MeOH/THF (5 mL/30 mL), 1 N HCl (20 mL) was added at rt. The resulting mixture was stirred at RT for 16 h, then concentrated and the residue was partitioned between EA (40 mL) and water (40 mL). The organic layer was dried over Na₂SO₄, filtered, and concentrated. The mixture was triturated with TBME (2×20 mL), filtered, and concentrated to afford the product as white solid (2 g). Yield: 49%. ESI-MS, m/z 388.0 (M+H)⁺.

### Step 4: Synthesis of 1-allyl-N,N-bis(4-methoxybenzyl)-1H-pyrazole-3-sulfonamide (63g)

To a solution of 63e (7.7 g, 20 mmol) in ACN (40 mL), 63f (2.7 g, 22 mmol), K₂CO₃ (5.5 g, 40 mmol) were added at rt. And then the resulting mixture was stirred at 70° C. for 16 h. Concentrated and the residue was partitioned between DCM (40 mL) and water (40 mL). The organic layers were dried over Na₂SO₄, filtered, concentrated. The organic layers were dried over Na₂SO₄, filtered. The mixture was concentrated and purified by FCC (25% EA/PE) to afford the product as yellow solid (5 g). Yield:
58%. ESI-MS, m/z 428.0 (M+H)⁺.

### Step 5: Synthesis of N,N-bis(4-methoxybenzyl)-1-(3-(4,4,5,5-tetramethyl-1,3,2-dioxaborolan-2-yl)propyl)-1H-pyrazole-3-sulfonamide (63h)

To a solution of 63g (5 g, 10.5 mmol) in DCM (40 mL), Chloro(1,5-cyclooctadiene)iridium(I) dimer (141 mg, 0.21 mmol), DPPE (2.1 g, 5.2 mmol), HBpin (1.6 g, 12.6 mmol) were added at RT under nitrogen. And then the resulting mixture was stirred at RT for 16 h. The mixture was washed by brine (30 mL), the organic layer was concentrated, and the residue was purified by FCC (35% EA/PE) to afford the product as yellow solid (4 g). Yield: 70%. ESI-MS, m/z 556.0 (M+H)⁺.

### Step 6: Synthesis of 1-(3-(4,4,5,5-tetramethyl-1,3,2-dioxaborolan-2-yl)propyl)-1H-pyrazole-3-sulfonamide. (63i)

To a solution of 63g (5 g, 9 mmol) in DCM (20 mL), TFA (10 mL) was added at rt. And then the resulting mixture was stirred at RT for 16 h. The mixture concentrated to afford the product as pale oil (2.3 g). Yield: 80%. ESI-MS, m/z 316.0 (M+H)⁺.

### Step 7: Synthesis of (3-(3-(N-((1,2,3,5,6,7-hexahydro-s-indacen-4-yl)carbamoyl)sulfamoyl)-1H-pyrazol-1-yl)propyl)boronic acid

The title compound was prepared following the procedures described in Example 29, Step 4 and Step 7, using 63i. The final product was purified by prep-HPLC (ACN:H₂O:NH₃·H₂O (0.05%), 10% to 40%) to afford the title compound as white solid (47.9 mg). ESI-MS, m/z 415.1 (M-OH)⁺.

### Example 64 and 65. Synthesis of N-((1,2,3,5,6,7-hexahydro-s-indacen-4-yl)carbamoyl)-1-(2-methyl-3-(4,4,5,5-tetramethyl-1,3,2-dioxaborolan-2-yl)propyl)-1H-pyrazole-3-sulfonamide and (3-(3-(N-((1,2,3,5,6,7-hexahydro-s-indacen-4-yl)carbamoyl)sulfamoyl)-1H-pyrazol-1-yl)-2-methylpropyl)boronic acid (65)

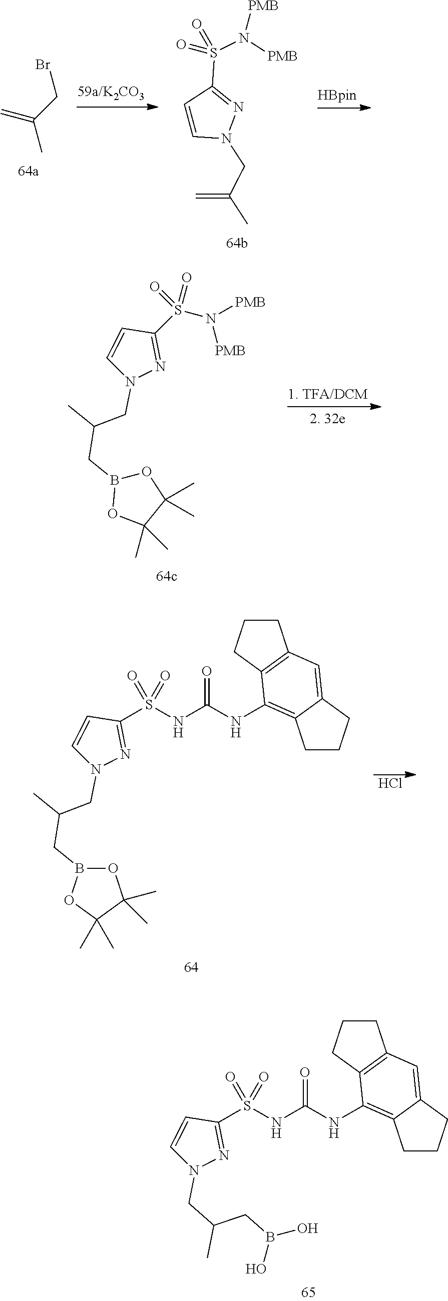

### Step 1: Synthesis of N,N-bis(4-methoxybenzyl)-1-(2-methylallyl)-1H-pyrazole-3-sulfonamide

Compound 64b was prepared following the procedure described in Example 59, Step 1, using 64a and 59a: ESI-MS, m/z 442.2 (M+H)⁺.

### Step 2: Synthesis of N,N-bis(4-methoxybenzyl)-1-(2-methyl-3-(4,4,5,5-tetramethyl-1,3,2-dioxaborolan-2-yl)propyl)-1H-pyrazole-3-sulfonamide

The title compound was prepared following the procedure described in Example 60, Step 2, using 64b instead of 60b: ESI-MS, m/z 570.3 (M+H)⁺.

### Step 3: Synthesis of N-((1,2,3,5,6,7-hexahydro-s-indacen-4-yl)carbamoyl)-1-(2-methyl-3-(4,4,5,5-tetramethyl-1,3,2-dioxaborolan-2-yl)propyl)-1H-pyrazole-3-sulfonamide (64)

The title compound was prepared following the procedure described in Example 60, Steps 3 and 4, using 64c instead of 60c: ESI-MS, m/z 529.3 (M+H)⁺.

### Step 4: Synthesis of (3-(3-(N-((1,2,3,5,6,7-hexahydro-s-indacen-4-yl)carbamoyl)sulfamoyl)-1H-pyrazol-1-yl)-2-methylpropyl)boronic acid (65)

A solution of 64 (15 mg) and MeB(OH)₂ (15 mg) in CH₃CN (2 mL) and HCl (1 N aq., 1 mL) was stirred at RT for 2 h. The reaction mixture was diluted with EtOAc. The organic layer was separated, concentrated and purified via reverse phase chromatography (CH₃CN (0.1% TFA)/water (0.1% TFA), 10˜100%) to afford 65 as white solid (5 mg): ESI-MS, m/z 469.2 (M+Na)⁺.

### Example 66. N-((1,2,3,5,6,7-hexahydro-s-indacen-4-yl)carbamoyl)-1-(2-methyl-2-(4,4,5,5-tetramethyl-1,3,2-dioxaborolan-2-yl)propyl)-1H-pyrazole-3-sulfonamide

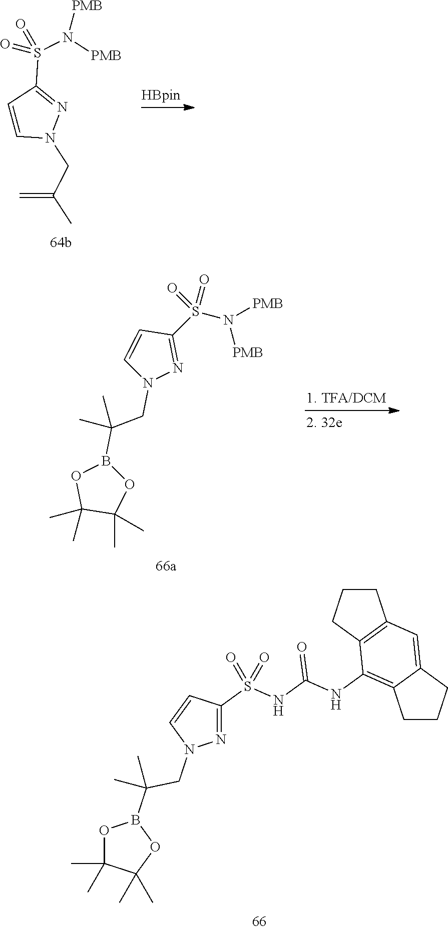

### Step 1: Synthesis of N,N-bis(4-methoxybenzyl)-1-(2-methyl-2-(4,4,5,5-tetramethyl-1,3,2-dioxaborolan-2-yl)propyl)-1H-pyrazole-3-sulfonamide (66a)

The title compound was prepared following the procedure described in Example 62, Step 1, using 64b instead of 62b: ESI-MS, m/z 570.2 (M+H)⁺.

### Step 2: Synthesis of N-((1,2,3,5,6,7-hexahydro-s-indacen-4-yl)carbamoyl)-1-(2-methyl-2-(4,4,5,5-tetramethyl-1,3,2-dioxaborolan-2-yl)propyl)-1H-pyrazole-3-sulfonamide (66)

The title compound was prepared following the procedure described in Example 60, Step 3 and 4, using 66a instead of 60c: ESI-MS, m/z 529.3 (M+H)⁺.

### Example 67. Synthesis of 3-(3-(N-((3,3-dimethyl-1,2,3,5,6,7-hexahydrodicyclopenta[b,e]pyridin-8-yl)carbamoyl)sulfamoyl)-1H-pyrazol-1-yl)propyl)boronic acid

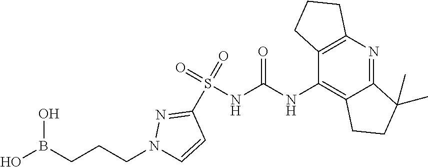 [SMILES: CC1(C)CCc2c1nc1c(c2NC(=O)NS(=O)(=O)c2ccn(CCCB(O)O)n2)CCC1]

The title compound was prepared following the procedures described in Example 63, utilizing 8-amino-3,3-dimethyl-1,2,3,5,6,7-hexahydrodicyclopenta[b,e]pyridine (PCT Int. Appl., 2020102100, 22 May 2020) in Step 7. The final product was purified by prep-HPLC (ACN:H₂O:NH₃·H₂O (0.05%), 10% to 40%) to afford as white solid. ESI-MS, m/z 462.2 (M+H)⁺.

### Example 68. Synthesis of (3-(3-(N-((4-cyano-3-fluoro-2,6-diisopropylphenyl)carbamoyl)sulfamoyl)-1H-pyrazol-1-yl)propyl)boronic acid

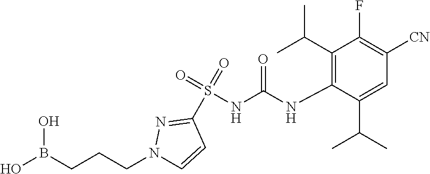 [SMILES: CC(C)c1cc(C#N)c(F)c(C(C)C)c1NC(=O)NS(=O)(=O)c1ccn(CCCB(O)O)n1]

The title compound was prepared following the procedures described in Example 63, using 2-fluoro-4-isocyanato-3,5-diisopropylbenzonitrile in Step 7. The final product was purified by prep-HPLC (ACN:H₂O:NH₃·H₂O (0.05%), 10% to 40%) to afford as white solid. ESI-MS, m/z 462.1 (M-OH)⁺.

### Example 69. Synthesis of (3-(3-(N-((5-(2-methoxypyridin-4-yl)-2,3-dihydro-1H-inden-4-yl)carbamoyl)sulfamoyl)-1H-pyrazol-1-yl)propyl)boronic acid

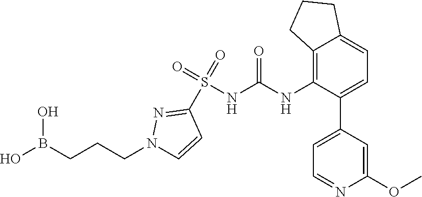 [SMILES: COc1cc(-c2ccc3c(c2NC(=O)NS(=O)(=O)c2ccn(CCCB(O)O)n2)CCC3)ccn1]

The title compound was prepared following the procedures described in Example 63, using 4-(4-isocyanato-2,3-dihydro-1H-inden-5-yl)-2-methoxypyridine in Step 7. The final product was purified by prep-HPLC (ACN:H₂O:NH₃·H₂O (0.05%), 10% to 40%) to afford as white solid. ESI-MS, m/z 500.1 (M+H)⁺.

### Example 70. Synthesis of N-((1,2,3,5,6,7-hexahydro-s-indacen-4-yl)carbamoyl)-1-(3-(4,4,5,5-tetramethyl-1,3,2-dioxaborolan-2-yl)propyl)cyclopropane-1-sulfonamide (70)

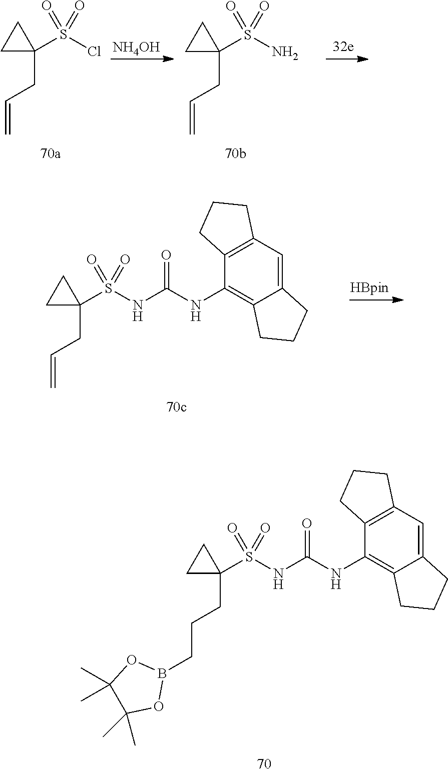

### Step 1: Synthesis of 1-allylcyclopropane-1-sulfonamide (70b)

NH₄OH (3 g, 28% aq., 24 mmol) was added dropwise to a solution of 70a (1 g, 5.6 mmol) in CH₃CN (2 mL) at 0° C. The mixture was warmed to RT for 20 h. The reaction was quenched with water and extracted with EtOAc. The organic layer was separated, washed with brine, dried over Na₂SO₄, and concentrated to give 72b as white solid (0.9 g): ESI-MS, m/z m/z 162.1 (M+H)⁺.

### Step 2: Synthesis of 1-allyl-N-((1,2,3,5,6,7-hexahydro-s-indacen-4-yl)carbamoyl)cyclopropane-1-sulfonamide (70c)

The title compound was prepared following the procedure described in Example 32, Step 3, using 70b: ESI-MS, m/z 361.2 (M+H)⁺.

### Step 3: Synthesis of N-((1,2,3,5,6,7-hexahydro-s-indacen-4-yl)carbamoyl)-1-(3-(4,4,5,5-tetramethyl-1,3,2-dioxaborolan-2-yl)propyl)cyclopropane-1-sulfonamide

Compound 70 was prepared following the procedure described in Example 32, Step 4, using 70c instead of 32d: ESI-MS, m/z 489.3 (M+H)⁺.

### Example 71. (3-(1-(N-((1,2,3,5,6,7-hexahydro-s-indacen-4-yl)carbamoyl)sulfamoyl)cyclopropyl)propyl)boronic acid

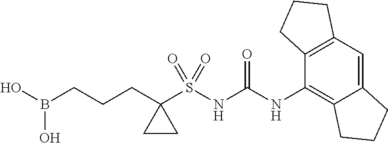 [SMILES: O=C(Nc1c2c(cc3c1CCC3)CCC2)NS(=O)(=O)C1(CCCB(O)O)CC1]

HCl (0.5 N, 0.2 mL) was added to a solution of 70 (15 mg) and MeB(OH)₂ (5 mg) in CH₃CN (1 mL). After the mixture was stirred at RT for 1 h, the reaction was quenched with water (0.2 mL), concentrated, and purified via reverse phase chromatography (CH₃CN (0.1% TFA)/water (0.1% TFA) 10˜100%) to afford 71 as white solid: ESI-MS, m/z 429.2 (M+Na)⁺.

### Example 72. (3-(4-(N-((1,2,3,5,6,7-hexahydro-s-indacen-4-yl)carbamoyl)sulfamoyl)piperidin-1-yl)propyl)boronic acid

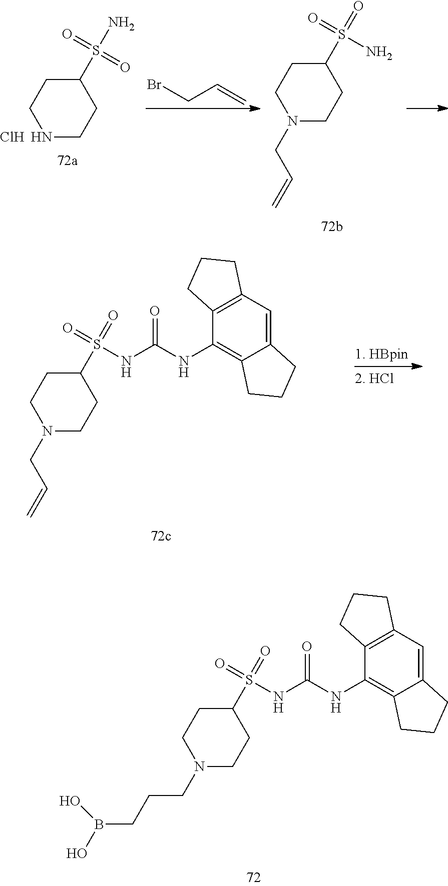

### Step 1: Synthesis of 1-allylpiperidine-4-sulfonamide (72b)

Compound 72b was prepared following the procedure described in Example 73, Step 1, using 72a instead of 73a, and was isolated as white solid: ESI-MS, m/z 205.2 (M+H)⁺.

### Step 2: Synthesis of 1-allyl-N-((1,2,3,5,6,7-hexahydro-s-indacen-4-yl)carbamoyl)piperidine-4-sulfonamide (72c)

The title compound was prepared following the procedure described in Example 32, Step 3, using 72b: ESI-MS, m/z 404.3 (M+H)⁺.

### Step 3: Synthesis of (3-(4-(N-((1,2,3,5,6,7-hexahydro-s-indacen-4-yl)carbamoyl)sulfamoyl)piperidin-1-yl)propyl)boronic acid

The title compound was prepared following the procedure described in Example 65, Steps 3 and 4, using 72c: ESI-MS, m/z 450.3 (M+H)⁺.

### Example 73. N-((1,2,3,5,6,7-hexahydro-s-indacen-4-yl)carbamoyl)-1-(3-(4,4,5,5-tetramethyl-1,3,2-dioxaborolan-2-yl)propyl)azetidine-3-sulfonamide

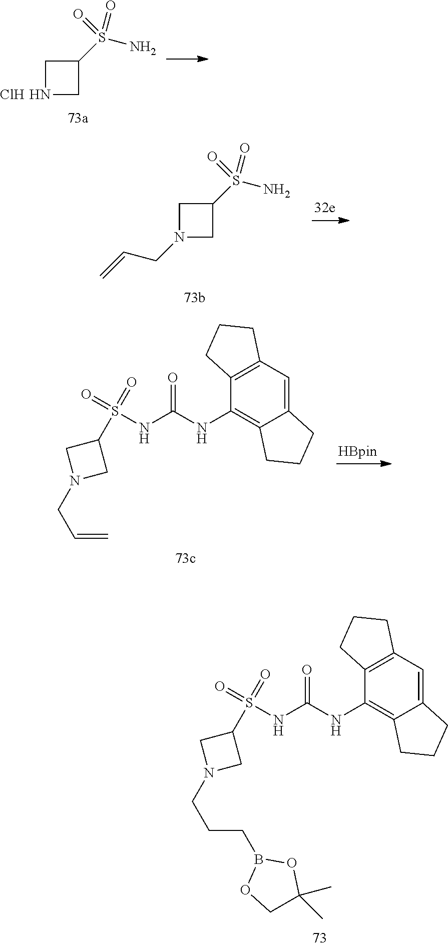 [SMILES: Cl ; NS(=O)(=O)C1CNC1 ; C=CCN1CC(S(N)(=O)=O)C1 ; C=CCN1CC(S(=O)(=O)NC(=O)Nc2c3c(cc4c2CCC4)CCC3)C1 ; CC1(C)COB(CCCN2CC(S(=O)(=O)NC(=O)Nc3c4c(cc5c3CCC5)CCC4)C2)O1]

### Step 1: Synthesis of 1-allylazetidine-3-sulfonamide (73b)

Allyl bromide (0.38 g, 3.2 mmol) was added dropwise to a mixture of 73a (0.5 g, 2.9 mmol) and K₂CO₃ (1.6 g, 11.6 mmol) in CH₃CN (8 mL) at 0° C. The mixture was warmed to RT for 40 h. The reaction was quenched with water and extracted with EtOAc. The organic layer was washed with brine and concentrated to give 73b: ESI-MS, m/z m/z 177.1.

### Step 2: Synthesis of 1-allyl-N-((1,2,3,5,6,7-hexahydro-s-indacen-4-yl)carbamoyl)azetidine-3-sulfonamide (73c)

The title compound was prepared following the procedure described in Example 32, Step 3, using 73b: ESI-MS, m/z 376.1 (M+H)⁺.

### Step 3: Synthesis of N-((1,2,3,5,6,7-hexahydro-s-indacen-4-yl)carbamoyl)-1-(3-(4,4,5,5-tetramethyl-1,3,2-dioxaborolan-2-yl)propyl)azetidine-3-sulfonamide

Compound 73 was prepared following the procedure described in Example 32, Step 4, using 73c instead of 32d: ESI-MS, m/z 504.1 (M+H)⁺.

### Biological Examples

### Biological Example I. IL-1β Inhibition Assay

### THP-1 Cell Culture Maintenance

Human monocyte-like THP-1 cells were grown in RPMI media supplemented with 10% FBS (growth media) at 37° C., 5% CO₂. Cell cultures were passaged every three days and maintained at a density of 5×10⁵ to 1×10⁶ cells per mL.

### Compound Sample Preparation.

Compounds were prepared as 10 mM stock solutions in 100% DMSO. For EC₅₀ determination, compounds were serially diluted 3-fold in 100% DMSO at 200× the desired final assay concentration. Each 200× dilution was then diluted 20-fold in growth media (step down dilution) to yield a 10× final assay concentration range. All compounds were tested over a final assay concentration range of 10 μM to 0.003 μM.

### Assay Procedure for the Detection of IL-1β in Cell Culture Supernatants.

The level of IL-10 in supernatants from PMA-differentiated THP-1 cells was determined by ELISA using either the R&D Systems Human IL-1 beta/IL-1F2 Quantikine ELISA or the cisbio HTRF human IL 1 beta kit. Manufacturer's protocols were followed without deviation.

The concentration of each compound which resulted in 50% reduction of IL-10 levels in the culture supernatants (EC₅₀) was determined. Background values were subtracted and resulting values were converted to percentage of vehicle-treated controls. Data was analyzed via GraphPad Prism 7.0 using a non-linear regression (variable slope (four-parameter)) equation.

Table 2 provides IL-10 ELISA EC₅₀ values for example compounds. A indicates EC₅₀>10 μM; B indicates EC₅₀ between 1 and 10 μM (inclusive); C indicates EC₅₀<1 μM.

### Biological Example II. Cell Viability Assessment

In order to assess the cytotoxicity of the compounds, an MTT cell viability assay was performed in either unstimulated THP-1 or HepG2 cells as follows. 5×10⁴ THP-1 or 5×10³ HepG2 cells were seeded into each well of a 96-well plate in 90 μL of media. Compounds were added to cells as described for the IL-10 assay and returned to the incubator for an additional 72 hours. Next, 25 μL of MTT solution (5 mg/mL in sterile PBS) was added to each well. Plates were incubated for 3 hours at 37° C., 5% CO₂. Next, for THP-1 cells, 100 μl of MTT solubilization solution (40% Dimethylformamide, 2% glacial acetic acid, 16% sodium dodecyl sulfate) was then added to each well and plates were incubated at room temperature in the dark overnight. For HepG2 cells, media was aspirated, 50 μL of DMSO was added and plates were mixed briefly with an orbital plate shaker. Absorbance at 570 nm was measured using a Perkin-Elmer EnVision plate reader.

The concentration of each compound which resulted in 50% reduction of cell viability (CC₅₀) was determined. Absorbance values were converted to percentage of vehicle-treated controls. Data was then analyzed using a non-linear regression (variable slope (four-parameter)) equation in GraphPad Prism 7.0.

Table 2 provides THP-1 cytotoxicity assay CC₅₀ values for example compounds. A indicates CC₅₀>10 μM; B indicates CC₅₀ between 1 and 10 μM (inclusive); C indicates CC₅₀<1 μM. NT, not tested.

TABLE 2
Biological testing data for example compounds.

| | IL-1β | THP-1 | HepG2 |
|---|---|---|---|
| Example | ELISA EC₅₀ | Cytotoxicity CC₅₀ | Cytotoxicity CC₅₀ |
| 1 | B | A | NT |
| 2 | B | A | NT |
| 3 | B | A | NT |
| 4 | A | A | NT |
| 5 | B | A | NT |
| 6 | B | A | NT |
| 7 | B | A | NT |
| 8 | A | A | NT |
| 9 | A | A | NT |
| 10 | A | A | NT |
| 11 | A | A | NT |
| 12 | B | A | NT |
| 13 | B | A | NT |
| 14 | B | A | NT |
| 15 | B | A | NT |
| 16 | B | A | NT |
| 17 | B | A | NT |
| 18 | A | A | NT |
| 19 | A | A | NT |
| 20 | A | A | NT |
| 21 | A | B | NT |
| 22 | A | A | NT |
| 23 | A | A | NT |
| 24 | B | A | NT |
| 25 | B | A | NT |
| 26 | C | A | NT |
| 27 | C | A | NT |
| 28 | C | A | NT |
| 29 | C | A | NT |
| 30 | B | A | NT |
| 31 | A | A | NT |
| 32 | B | A | NT |
| 33 | B | A | NT |
| 34 | B | A | NT |
| 35 | A | A | NT |
| 36 | A | A | NT |
| 37 | B | A | NT |
| 38 | B | A | NT |
| 39 | B | A | NT |
| 40 | B | A | NT |
| 41 | B | A | NT |
| 42 | C | A | NT |
| 43 | B | A | NT |
| 44 | B | A | NT |
| 45 | B | A | NT |
| 46 | B | A | NT |
| 47 | B | A | NT |
| 48 | B | A | NT |
| 49 | B | A | NT |
| 50 | B | A | NT |
| 51 | B | A | NT |
| 52 | A | A | A |
| 53 | B | A | A |
| 54 | A | A | A |
| 55 | C | A | A |
| 56 | B | A | A |
| 57 | B | A | A |
| 58 | C | NT | A |
| 59 | C | A | NT |
| 60 | B | A | A |
| 61 | C | A | A |
| 62 | C | A | A |
| 63 | C | NT | A |
| 64 | C | NT | A |
| 65 | C | NT | A |
| 66 | C | NT | A |
| 67 | C | NT | A |
| 68 | C | NT | A |
| 69 | C | NT | A |
| 70 | B | NT | A |
| 71 | B | NT | A |
| 72 | B | NT | A |
| 73 | C | NT | A |
| MCC-950 | C | A | NT |

1. A compound of Formula (I), or a pharmaceutically acceptable salt, solvate, or stereoisomer thereof: 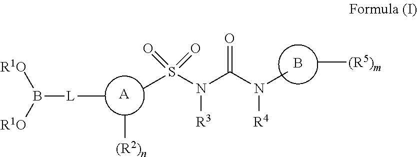
wherein:
Ring A is cycloalkyl, heterocycloalkyl, aryl, or heteroaryl;
L is a bond or a C₁-C₆ alkylene optionally substituted with one, two, or three R^{L};
each R^{L} is independently deuterium, halogen, —CN, —OH, —OW, —SH, —SW, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{L} on the same carbon are taken together to form an oxo;
each R¹ is independently hydrogen or C₁-C₆ alkyl;
or two R¹ are taken together to form a heterocycloalkyl optionally substituted with one, two, three, four, or five R^{1a};
each R^{1a} is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₁-C₆alkylene(COOH), C₂-C₆alkenyl, C₂-C₆alkynyl, cycloalkyl, heterocycloalkyl, aryl, or heteroaryl;
or two R^{1a} on the same carbon are taken together to form an oxo;
each R² is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally substituted with one, two, or three R^{2a};
each R^{2a} is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{2a} on the same carbon are taken together to form an oxo;
n is 0-3;
or one R¹ and one R² are taken together to form a heterocycloalkyl optionally substituted with one, two, or three R¹⁻²;
each R¹⁻² is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R¹⁻² on the same carbon are taken together to form an oxo;
R³ and R⁴ are each independently hydrogen or C₁-C₆ alkyl;
Ring B is cycloalkyl, heterocycloalkyl, aryl, or heteroaryl;
each R⁵ is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally substituted with one, two, or three R^{5a};
or two R⁵ on adjacent atoms are taken together to form a cycloalkyl, heterocycloalkyl, aryl, or heteroaryl;
each optionally substituted with one, two, or three R^{b};
each R^{5a} is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{5a} on the same carbon are taken together to form an oxo;
each R^{b} is independently deuterium, halogen, —CN, —OH, —OW, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{b} on the same carbon are taken together to form an oxo;
m is 0-5;
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl);
wherein each alkyl, alkenyl, alkynyl, cycloalkyl, heterocycloalkyl, aryl, and heteroaryl is independently optionally substituted with one, two, or three deuterium, oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆deuteroalkyl, C₁-C₆haloalkyl, C₁-C₆hydroxyalkyl, or C₁-C₆aminoalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl);
wherein each alkyl, alkenyl, alkynyl, cycloalkyl, heterocycloalkyl, aryl, and heteroaryl is independently optionally substituted with one, two, or three deuterium, oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆deuteroalkyl, C₁-C₆hydroxyalkyl, or C₁-C₆aminoalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one, two, or three oxo, deuterium, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆deuteroalkyl, C₁-C₆hydroxyalkyl, or C₁-C₆aminoalkyl;
or R^{c} and R^{d} are taken together with the atom to which they are attached to form a heterocycloalkyl optionally substituted with one, two, or three oxo, deuterium, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆deuteroalkyl, C₁-C₆hydroxyalkyl, or C₁-C₆aminoalkyl.

2. The compound of claim 1, or a pharmaceutically acceptable salt, solvate, or stereoisomer thereof, wherein:
Ring A is phenyl or a 5- or 6-membered heteroaryl.

3. The compound of claim 1 or 2, or a pharmaceutically acceptable salt, solvate, or stereoisomer thereof, wherein:
Ring A is phenyl.

4. The compound of claim 1 or 2, or a pharmaceutically acceptable salt, solvate, or stereoisomer thereof, wherein:
Ring A is pyridinyl.

5. The compound of claim 1 or 2, or a pharmaceutically acceptable salt, solvate, or stereoisomer thereof, wherein:
Ring A is a 5-membered heteroaryl.

6. The compound of any one of claims 1-5, or a pharmaceutically acceptable salt, solvate, or stereoisomer thereof, wherein:
each R¹ is hydrogen.

7. The compound of any one of claims 1-5, or a pharmaceutically acceptable salt, solvate, or stereoisomer thereof, wherein:
two R¹ are taken together to form a heterocycloalkyl optionally substituted with one, two, three, four, or five R^{1a}.

8. The compound of any one of claims 1-5 or 7, or a pharmaceutically acceptable salt, solvate, or stereoisomer thereof, wherein:
two R¹ are taken together to form 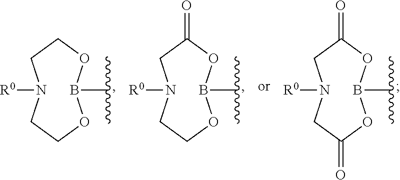
wherein R⁰ is hydrogen, C₁-C₆alkyl, C₁-C₆hydroxyalkyl, or C₁-C₆alkylene(COOH).

9. The compound of any one of claims 1-8, or a pharmaceutically acceptable salt, solvate, or stereoisomer thereof, wherein:
L is C₁-C₂ alkylene.

10. The compound of any one of claims 1-8, or a pharmaceutically acceptable salt, solvate, or stereoisomer thereof, wherein:
L is a bond.

11. The compound of any one of claims 1-10, or a pharmaceutically acceptable salt, solvate, or stereoisomer thereof, wherein:
each R² is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl.

12. The compound of any one of claims 1-11, or a pharmaceutically acceptable salt, solvate, or stereoisomer thereof, wherein:
each R² is independently halogen, —CN, —OH, —OR^{a}, —NR^{c}R^{d}, C₁-C₆alkyl, C₁-C₆haloalkyl, C₁-C₆deuteroalkyl, cycloalkyl, or heterocycloalkyl.

13. The compound of any one of claims 1-12, or a pharmaceutically acceptable salt, solvate, or stereoisomer thereof, wherein:
each R² is independently halogen, C₁-C₆alkyl, or cycloalkyl.

14. The compound of any one of claims 1-13, or a pharmaceutically acceptable salt, solvate, or stereoisomer thereof, wherein:
n is 0-2.

15. The compound of any one of claims 1-14, or a pharmaceutically acceptable salt, solvate, or stereoisomer thereof, wherein:
one R¹ and one R² are taken together to form a heterocycloalkyl optionally substituted with one, two, or three R¹⁻².

16. The compound of any one of claims 1-15, or a pharmaceutically acceptable salt, solvate, or stereoisomer thereof, wherein:
one R¹ and one R² are taken together to form a 5- or 6-membered heterocycloalkyl optionally substituted with one, two, or three R¹⁻².

17. The compound of any one of claims 1-16, or a pharmaceutically acceptable salt, solvate, or stereoisomer thereof, wherein:
one R¹ and one R² are taken together to form a 5- or 6-membered heterocycloalkyl optionally substituted with one, two, or three R¹⁻²; wherein the 5- or 6-membered heterocycloalkyl is 1,2-oxaborolane or 1,2-oxaborinane.

18. The compound of any one of claims 1-17, or a pharmaceutically acceptable salt, solvate, or stereoisomer thereof, wherein:

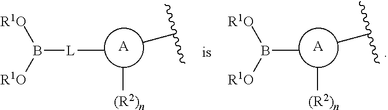

19. The compound of any one of claims 1-17, or a pharmaceutically acceptable salt, solvate, or stereoisomer thereof, wherein: 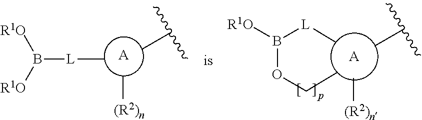
wherein p is 1-3 and n′ is 0-2.

20. The compound of any one of claims 1-19, or a pharmaceutically acceptable salt, solvate, or stereoisomer thereof, wherein:
R³ is hydrogen.

21. The compound of any one of claims 1-20, or a pharmaceutically acceptable salt, solvate, or stereoisomer thereof, wherein:
R⁴ is hydrogen.

22. The compound of any one of claims 1-21, or a pharmaceutically acceptable salt, solvate, or stereoisomer thereof, wherein:
Ring B is aryl.

23. The compound of any one of claims 1-22, or a pharmaceutically acceptable salt, solvate, or stereoisomer thereof, wherein:
each R⁵ is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is optionally substituted with one, two, or three R^{5a}.

24. The compound of any one of claims 1-23, or a pharmaceutically acceptable salt, solvate, or stereoisomer thereof, wherein:
each R⁵ is independently halogen, —CN, —OH, —OR^{a}, —NR^{c}R^{d}, C₁-C₆alkyl, C₁-C₆haloalkyl, C₁-C₆deuteroalkyl, cycloalkyl, or heterocycloalkyl; wherein each alkyl, cycloalkyl, and heterocycloalkyl is optionally substituted with one, two, or three R^{5a}.

25. The compound of any one of claims 1-24, or a pharmaceutically acceptable salt, solvate, or stereoisomer thereof, wherein:
each R⁵ is independently halogen or C₁-C₆alkyl.

26. The compound of any one of claims 1-25, or a pharmaceutically acceptable salt, solvate, or stereoisomer thereof, wherein:
each R⁵ is independently C₁-C₆alkyl.

27. The compound of any one of claims 1-26, or a pharmaceutically acceptable salt, solvate, or stereoisomer thereof, wherein:
each R⁵ is independently halogen, C₁-C₆alkyl, or heteroaryl; wherein each alkyl heteroaryl is optionally substituted with one, two, or three R^{5a}.

28. The compound of any one of claims 1-27, or a pharmaceutically acceptable salt, solvate, or stereoisomer thereof, wherein:
two R⁵ on adjacent atoms are taken together to form a cycloalkyl optionally substituted with one, two, or three R^{5b}.

29. The compound of any one of claims 1-28, or a pharmaceutically acceptable salt, solvate, or stereoisomer thereof, wherein:
m is 0-4.

30. The compound of any one of claims 1-29, or a pharmaceutically acceptable salt, solvate, or stereoisomer thereof, wherein:
m is 2-4.

31. A compound of Formula (II), or a pharmaceutically acceptable salt, solvate, or stereoisomer thereof: 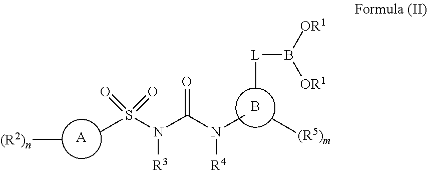
wherein:
Ring A is cycloalkyl, heterocycloalkyl, aryl, or heteroaryl;
each R² is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally substituted with one, two, or three R^{2a};
each R^{2a} is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{2a} on the same carbon are taken together to form an oxo;
n is 0-3;
R³ and R⁴ are each independently hydrogen or C₁-C₆ alkyl;
Ring B is cycloalkyl, heterocycloalkyl, aryl, or heteroaryl;
L is a bond or a C₁-C₆ alkylene optionally substituted with one, two, or three R^{L};
each R^{L} is independently deuterium, halogen, —CN, —OH, —OW, —SH, —SW, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{L} on the same carbon are taken together to form an oxo;
each R¹ is independently hydrogen or C₁-C₆ alkyl;
or two R¹ are taken together to form a heterocycloalkyl optionally substituted with one, two, three, four, or five R^{1a};
each R^{1a} is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₁-C₆alkylene(COOH), C₂-C₆alkenyl, C₂-C₆alkynyl, cycloalkyl, heterocycloalkyl, aryl, or heteroaryl;
or two R^{1a} on the same carbon are taken together to form an oxo;
or one R¹ and one R⁵ are taken together to form a heterocycloalkyl optionally substituted with one, two, or three R¹⁻⁵;
each R¹⁻⁵ is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R¹⁻⁵ on the same carbon are taken together to form an oxo;
each R⁵ is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally substituted with one, two, or three R^{a};
or two R⁵ on adjacent atoms are taken together to form a cycloalkyl, heterocycloalkyl, aryl, or heteroaryl;
each optionally substituted with one, two, or three R^{5b};
each R^{5a} is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{5a} on the same carbon are taken together to form an oxo;
each R^{5b} is independently deuterium, halogen, —CN, —OH, —OR^{a}, —SH, —SR^{a}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{5b} on the same carbon are taken together to form an oxo;
m is 0-4;
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl);
wherein each alkyl, alkenyl, alkynyl, cycloalkyl, heterocycloalkyl, aryl, and heteroaryl is independently optionally substituted with one, two, or three deuterium, oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆deuteroalkyl, C₁-C₆haloalkyl, C₁-C₆hydroxyalkyl, or C₁-C₆aminoalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl);
wherein each alkyl, alkenyl, alkynyl, cycloalkyl, heterocycloalkyl, aryl, and heteroaryl is independently optionally substituted with one, two, or three deuterium, oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆deuteroalkyl, C₁-C₆hydroxyalkyl, or C₁-C₆aminoalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one, two, or three oxo, deuterium, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆deuteroalkyl, C₁-C₆hydroxyalkyl, or C₁-C₆aminoalkyl;
or R^{c} and R^{d} are taken together with the atom to which they are attached to form a heterocycloalkyl optionally substituted with one, two, or three oxo, deuterium, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆deuteroalkyl, C₁-C₆hydroxyalkyl, or C₁-C₆aminoalkyl;
provided that the compound of Formula (II) is not

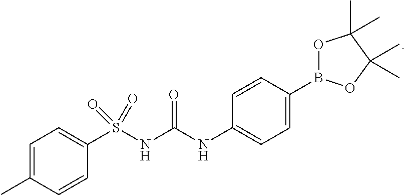

32. The compound of claim 31, or a pharmaceutically acceptable salt, solvate, or stereoisomer thereof, wherein:
Ring A is phenyl or 5- or 6-membered heteroaryl.

33. The compound of claim 31 or 32, or a pharmaceutically acceptable salt, solvate, or stereoisomer thereof, wherein:
Ring A is phenyl.

34. The compound of claim 31 or 32, or a pharmaceutically acceptable salt, solvate, or stereoisomer thereof, wherein:
Ring A is pyridinyl.

35. The compound of claim 31 or 32, or a pharmaceutically acceptable salt, solvate, or stereoisomer thereof, wherein:
Ring A is a 5-membered heteroaryl.

36. The compound of any one of claims 31-35, or a pharmaceutically acceptable salt, solvate, or stereoisomer thereof, wherein:
each R² is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl.

37. The compound of any one of claims 31-36, or a pharmaceutically acceptable salt, solvate, or stereoisomer thereof, wherein:
each R² is independently halogen, —CN, —OH, —OR^{a}, —NR^{c}R^{d}, C₁-C₆alkyl, C₁-C₆haloalkyl, C₁-C₆deuteroalkyl, cycloalkyl, or heterocycloalkyl.

38. The compound of any one of claims 31-37, or a pharmaceutically acceptable salt, solvate, or stereoisomer thereof, wherein:
each R² is independently halogen, C₁-C₆alkyl, or cycloalkyl.

39. The compound of any one of claims 31-38, or a pharmaceutically acceptable salt, solvate, or stereoisomer thereof, wherein:
n is 0-2.

40. The compound of any one of claims 31-39, or a pharmaceutically acceptable salt, solvate, or stereoisomer thereof, wherein:
R³ is hydrogen.

41. The compound of any one of claims 31-40, or a pharmaceutically acceptable salt, solvate, or stereoisomer thereof, wherein:
R⁴ is hydrogen.

42. The compound of any one of claims 31-41, or a pharmaceutically acceptable salt, solvate, or stereoisomer thereof, wherein:
Ring B is aryl.

43. The compound of any one of claims 31-42, or a pharmaceutically acceptable salt, solvate, or stereoisomer thereof, wherein:
each R¹ is hydrogen.

44. The compound of any one of claims 31-43, or a pharmaceutically acceptable salt, solvate, or stereoisomer thereof, wherein:
two R¹ are taken together to form a heterocycloalkyl optionally substituted with one, two, three, four, or five R^{1a}.

45. The compound of any one of claims 31-44, or a pharmaceutically acceptable salt, solvate, or stereoisomer thereof, wherein:
two R¹ are taken together to form 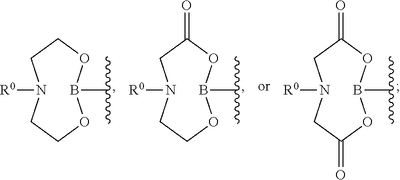
wherein R⁰ is hydrogen, C₁-C₆alkyl, C₁-C₆hydroxyalkyl, or C₁-C₆alkylene(COOH).

46. The compound of any one of claims 31-45, or a pharmaceutically acceptable salt, solvate, or stereoisomer thereof, wherein:
L is C₁-C₂ alkylene.

47. The compound of any one of claims 31-46, or a pharmaceutically acceptable salt, solvate, or stereoisomer thereof, wherein:
L is a bond.

48. The compound of any one of claims 31-47, or a pharmaceutically acceptable salt, solvate, or stereoisomer thereof, wherein:
each R⁵ is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is optionally substituted with one, two, or three R^{5a}.

49. The compound of any one of claims 31-48, or a pharmaceutically acceptable salt, solvate, or stereoisomer thereof, wherein:
each R⁵ is independently halogen, —CN, —OH, —OR^{a}, —NR^{c}R^{d}, C₁-C₆alkyl, C₁-C₆haloalkyl, C₁-C₆deuteroalkyl, cycloalkyl, or heterocycloalkyl; wherein each alkyl, cycloalkyl, and heterocycloalkyl is optionally substituted with one, two, or three R^{5a}.

50. The compound of any one of claims 31-49, or a pharmaceutically acceptable salt, solvate, or stereoisomer thereof, wherein:
each R⁵ is independently halogen or C₁-C₆alkyl.

51. The compound of any one of claims 31-50, or a pharmaceutically acceptable salt, solvate, or stereoisomer thereof, wherein:
each R⁵ is independently halogen, C₁-C₆alkyl, or heteroaryl; wherein each alkyl heteroaryl is optionally substituted with one, two, or three R^{5a}.

52. The compound of any one of claims 31-51, or a pharmaceutically acceptable salt, solvate, or stereoisomer thereof, wherein:
each R⁵ is independently C₁-C₆alkyl.

53. The compound of any one of claims 31-52, or a pharmaceutically acceptable salt, solvate, or stereoisomer thereof, wherein:
two R⁵ on adjacent atoms are taken together to form a cycloalkyl optionally substituted with one, two, or three R^{5b}.

54. The compound of any one of claims 31-53, or a pharmaceutically acceptable salt, solvate, or stereoisomer thereof, wherein:
m is 0-4.

55. The compound of any one of claims 31-54, or a pharmaceutically acceptable salt, solvate, or stereoisomer thereof, wherein:
m is 2-4.

56. The compound of any one of claims 31-55, or a pharmaceutically acceptable salt, solvate, or stereoisomer thereof, wherein:
one R¹ and one R⁵ are taken together to form a heterocycloalkyl optionally substituted with one, two, or three R¹⁻⁵.

57. The compound of any one of claims 31-56, or a pharmaceutically acceptable salt, solvate, or stereoisomer thereof, wherein:
one R¹ and one R⁵ are taken together to form a 5- or 6-membered heterocycloalkyl optionally substituted with one, two, or three R¹⁻⁵.

58. The compound of any one of claims 31-57, or a pharmaceutically acceptable salt, solvate, or stereoisomer thereof, wherein:
one R¹ and one R⁵ are taken together to form a 5- or 6-membered heterocycloalkyl optionally substituted with one, two, or three R¹⁻⁵; wherein the 5- or 6-membered heterocycloalkyl is 1,2-oxaborolane or 1,2-oxaborinane.

59. The compound of any one of claims 31-58, or a pharmaceutically acceptable salt, solvate, or stereoisomer thereof, wherein:

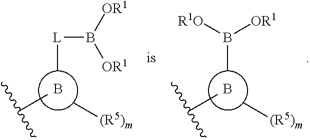

60. The compound of any one of claims 31-58, or a pharmaceutically acceptable salt, solvate, or stereoisomer thereof; wherein: 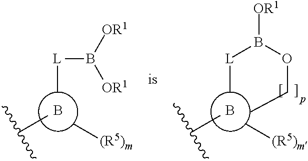
wherein p is 1-3 and m′ is 0-3.

61. The compound of any one of claims 1-60, or a pharmaceutically acceptable salt, solvate, or stereoisomer thereof; wherein the compound is selected from a compound found in Table 1.

62. A pharmaceutical composition comprising a compound of any one of claims 1-61, or a pharmaceutically acceptable salt, solvate, or stereoisomer thereof, and a pharmaceutically acceptable excipient.

63. A method of treating of a disease which is responsive to inhibition of activation of the NLRP3 inflammasome in a subject in need thereof, the method comprising administering to the subject an effective amount of a compound of any one of claims 1-61, or a pharmaceutically acceptable salt, solvate, or stereoisomer thereof.

64. A method of treating of a disease which is responsive to modulation of one or more of IL-6, IL-1 beta, IL-17, IL-18, IL-1 alpha, IL-37, IL-22, IL-33 and Th17 cells in a subject in need thereof, the method comprising administering to the subject an effective amount of a compound of any one of claims 1-61, or a pharmaceutically acceptable salt, solvate, or stereoisomer thereof.

65. The method of claim 63 or 64, wherein the disease is an immune system disease, an inflammatory disease, an autoimmune disease, a skin disease, a cardiovascular disease, cancer, a renal system disease, a gastro-intestinal tract disease, a respiratory system disease, a disease of the endocrine system, a viral disease, or a central nervous system (CNS) disease.

66. The method of claim 63 or 64, wherein the disease is constitutive inflammation including viral- or pathogen-associated hyperinflammation, cytokine release syndrome, acute respiratory distress syndrome, acute lung injury, septic shock, macrophage activating syndrome, hemophagocytic lymphohistiocytosis, coronavirus associated disease, cryopyrin-associated periodic syndromes (CAPS), Muckle-Wells syndrome (MWS), familial cold autoinflammatory syndrome (FCAS) and neonatal-onset multisystem inflammatory disease (NOMID), autoinflammatory diseases, familial Mediterranean fever (FMF), TNF receptor associated periodic syndrome (TRAPS), mevalonate kinase deficiency (MKD), hyperimmunoglobulinemia D and periodic fever syndrome (HIDS), deficiency of interleukin I receptor (DIRA) antagonist, Majeed syndrome, pyogenic arthritis, pyoderma gangrenosum and acne syndrome (PAPA), haploinsufficiency of A20 (HA20), pediatric granulomatous arthritis (PGA), PLCG2-associated antibody deficiency and immune dysregulation (PLAID), PLCG2-associated autoinflammation, antibody deficiency and immune dysregulation (APLAID) and sideroblastic anemia with B-cell immunodeficiency, periodic fevers, and developmental delay (SIFD); autoimmune diseases including multiple sclerosis (MS), type-1 diabetes, psoriasis, rheumatoid arthritis, Behcet's disease, Sjogren's syndrome and Schnitzler syndrome; macrophage activation syndrome; Blau syndrome; respiratory diseases including chronic obstructive pulmonary disorder (COPD), asthma such as allergic asthma and steroid-resistant asthma, asbestosis, silicosis and cystic fibrosis; dermatitis including contact dermatitis; central nervous system diseases including Parkinson's disease, Alzheimer's disease, motor neuron disease, Huntington's disease, cerebral malaria and brain injury from pneumococcal meningitis; metabolic diseases including Type 2 diabetes, atherosclerosis, obesity, gout, pseudo-gout; ocular diseases including those of the ocular epithelium, age-related macular degeneration (AMD), uveitis, corneal infection and dry eye; kidney disease including chronic kidney disease, oxalate nephropathy, nephrocalcinosis and diabetic nephropathy; liver disease including non-alcoholic steatohepatitis (NASH) and alcoholic liver disease; inflammatory reactions in skin including contact hypersensitivity and sunburn; inflammatory reactions in the joints including osteoarthritis, systemic juvenile idiopathic arthritis, adult-onset Still's disease, relapsing polychondritis; viral infections including alpha virus (Chikungunya, Ross River) and flavivirus (Dengue, Zika), flu, HIV; hidradenitis suppurativa (HS) and other cyst-causing skin diseases; cancers including lung cancer metastasis, pancreatic cancers, gastric cancers, myelodysplastic syndrome, leukemia; polymyositis; stroke including ischemic stroke; myocardial infarction including recurrent myocardial infarction; congestive heart failure; embolism; cardiovascular disease; Graft versus Host Disease; hypertension; colitis; helminth infection; bacterial infection; abdominal aortic aneurism; wound healing; depression, psychological stress; ischemia reperfusion injury, or diseases where an individual has been determined to carry a germline or somatic non-silent mutation in NLRP3.
